(19) United States
(12) Patent Application Publication
(10) Pub. No.: US 2026/0194524 A1
(43) Pub. Date: Jul. 9, 2026

(54) ANTI-MICROBIAL ANTIBODY SIGNATURES RELATED TO LUNG TUMORS AND USES THEREOF

(71) Applicant: ARIZONA BOARD OF REGENTS ON BEHALF OF ARIZONA STATE UNIVERSITY, Scottsdale, AZ (US)

(72) Inventors: Mahasish SHOME, Tempe, AZ (US); Ji QIU, Chandler, AZ (US); Yunro CHUNG, Chandler, AZ (US); Joshua LABAER, Chandler, AZ (US)

(21) Appl. No.: 19/105,756

(22) PCT Filed: Aug. 28, 2023
(86) PCT No.: PCT/US2023/073029

§ 371 Date: Feb. 21, 2025

Related U.S. Application Data
(60) Provisional application No. 63/401,410, filed on Aug. 26, 2022.

(51) Int. Cl. G01N 33/5752

(52) U.S. Cl. CPC G01N 33/5752; G01N 2333/045; G01N 2333/05; G01N 2333/075; G01N 2333/11; G01N 2333/165; G01N 2333/195; G01N 2333/205; G01N 2333/21; G01N 2333/285; G01N 2333/315; G01N 2333/3156; G01N 2333/35

(57) ABSTRACT

The present invention relates anti-microbial antibody signatures related to lung tumors, including benign tumors and adenocarcinomas. Disclosed herein are antibody panels comprising antigens from bacteria and/or viruses for use in early and accurate diagnosis of lung cancer and for reducing false positive lung carcinoma diagnoses from CT screening.

### CROSS-REFERENCE TO RELATED APPLICATIONS

This application is a national stage of International Application PCT/US2023/073029, titled “ANTI-MICROBIAL ANTIBODY SIGNATURES RELATED TO LUNG TUMORS AND USES THEREOF,” filed Aug. 28, 2023, which claims the benefit of U.S. Provisional Application No. 63/401,410, entitled “Anti-Microbial Antibody Signatures Related to Lung Tumors and Uses Thereof,” which was filed Aug. 26, 2022, the entire disclosures of which are hereby incorporated herein by this reference.

### STATEMENT REGARDING FEDERALLY SPONSORED RESEARCH OR DEVELOPMENT

This invention was made with government support under R01 CA199948 and U01 CA214201 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 194,309 bytes XML file named “11157-131_SeqList” created on Feb. 26, 2026.

### FIELD OF THE INVENTION

The invention relates to anti-microbial antibody signatures of lung tumors and their use in early and accurate diagnosis of lung cancer.

### BACKGROUND OF THE INVENTION

Cancers attributable to infections have a greater incidence than any individual type of cancer worldwide. Lung cancer is the leading cause of cancer death among men and women. Although risk factors like smoking are well established, lung cancer among never-smokers is the 6th most common cause of cancer death in the United States and most smokers never get lung cancer. Clearly, other factors contribute to carcinogenesis.

Computer Tomography (CT) screening can detect lung cancer early, when the cancer is most treatable, and reduce mortality. However, this diagnostic method suffers a high false positive rate. CT. Only approximately 4 percent of patients with a positive finding of indeterminate pulmonary nodules (IPN) by CT scan were found to have lung cancer. Most inflammatory lung nodules are due to microbial infections that are difficult to distinguish from lung cancer. Thus, there is a need for molecular biomarkers that can distinguish malignant and benign indeterminate pulmonary nodules (IPN) detected by CT scan.

### SUMMARY OF THE INVENTION

Disclosed herein are antibody panels for diagnosing if a subject has lung carcinoma and for distinguishing between malignant and benign indeterminate pulmonary nodules. Also disclosed is a polypeptide having an amino acid sequence selected from SEQ ID NO. 61-120, wherein the polypeptide may be used to screen for antibodies in a subject that is likely to be a smoker, have a benign module, or have lung carcinoma. In some aspects, isolated nucleic acid sequence having at least 80% sequence identity to a sequence selected from SEQ ID NOs. 1-60 and encodes an amino acid sequence set forth in SEQ ID NOs. 61-120 are disclosed. Accordingly, also disclosed are methods of diagnosing lung carcinoma in a subject and methods of reducing false positives in diagnosing lung carcinoma using CT scans using the aforementioned polypeptide or isolated nucleic acid sequence.

In some aspects, the disclosure concerns antibody panels for diagnosing whether a subject has lung carcinoma, the antibody panel comprising at least one antigen from at least one bacterium selected from the group consisting of: Helicobacter pylori, Pseudomonas aeruginosa, Streptococcus pneumoniae, Haemophilus influenzae, Streptococcus pyogenes, Mycobacterium tuberculosis, Streptococcus gallolyticus, Fusobacterium nucleatum, Klebsiella pneumoniae, Escherichia coli, Gemella haemolysans, Bacteroides vulgatus, Campylobacter jejuni, Bacteroides fragilis, Peptostreptococcus anaerobius, Citrobacter koseri, Leptotrichia buccalis, Clostridium difficile, Enterococcus faecalis, Porphyromonas gingivalis, Bifidobacterium adolescentis, Prevotella copri, Faecalibacterium prausnitzii, and Veillonella parvula.

In some embodiments, the antibody panel further comprises at least one antigen from at least one virus selected from the group consisting of: human herpesvirus, human mastadenovirus, influenza A virus, human coronavirus OC43, enterovirus, rhinovirus B, human parainfluenza virus 1, human metapneumovirus, human respiratory syncytial virus B, and human coronavirus 229E.

In certain embodiments, the virus is selected from the group consisting of: human herpesvirus 4, human mastadenovirus C, influenza A virus, human herpesvirus 5, human mastadenovirus F, human coronavirus OC43, enterovirus C, human mastadenovirus B, rhinovirus B, human parainfluenza virus 1, human metapneumovirus, human respiratory syncytial virus B, human coronavirus 229E, and enterovirus D.

In some embodiments, the antibody panel comprises at least one antigen from at least one bacterium selected from the group consisting of: Campylobacter jejuni, Enterococcus faecalis, Helicobacter pylori, Pseudomonas aeruginosa, Streptococcus pneumoniae, Haemophilus influenzae, Streptococcus pyogenes, Mycobacterium tuberculosis, Streptococcus gallolyticus, and Fusobacterium nucleatum.

Certain antibody panels comprise at least one antigen from at least one virus selected from the group consisting of: human herpesvirus 4, human mastadenovirus C, influenza A virus, human herpesvirus 5, human mastadenovirus F, and Human coronavirus OC43.

Some antibody panels comprise at least 20 antigens. In some embodiments, the antibody panel comprises at least 20 antigens having the amino acid sequence set forth in SEQ ID NOs. 61-120. In some aspects, the at least 20 antigens are encoded by the nucleotide sequences set forth in SEQ ID NOs. 1-60. In other embodiments, the antibody panel comprises antigens detected by the antibodies SgCD00783211, PaCD00632112, HpCD00781518, HhCD00595176, HpCD00780533, HpCD00781139, GhCD00811667, SpCD00810996, SpCD00876258, PaCD00812634, SpCD00810910, HpCD00781182, IaCD00844155, HpCD00781050, HsCD00959752, HpCD00780821, HpCD00780927, CjCD00811525, SpCD00876160, and HhCD00595190, have the amino acid sequences set forth in SEQ ID Nos. 61-80, or encoded by the nucleotide sequences set forth in SEQ ID NOs. 1-20.

In yet other embodiments, the antibody panel comprises antigens detected by the antibodies SpCD00818023, SgCD00783211, VzCD00594955, HhCD00594867, PaCD00632112, HrCD00959476, HaCD00953227, SgCD00811573, HhCD00595176, SpCD00818032, HpCD00780240, HpCD00780836, HmCD00952743, HmCD00849349, MtCD00544325, SpCD00875948, HpCD00781213, HpCD00849291, HhCD00595168, and SpCD00810863, have the amino acid sequences set forth in SEQ ID Nos. 81-100, or encoded by the nucleotide sequences set forth in SEQ ID NOs. 21-40.

Still other embodiments, have an antibody panel comprising antigens detected by the antibodies HsCD00959791, HmCD00849346, PaCD00632105, HmCD00849349, SpCD00818023, IcCD00953076, HmCD00956407, HpCD00780836, MtCD00412192, HmCD00849278, SpCD00876414, HpCD00781182, HrCD00959475, HpCD00781725, HpCD00953108, HpCD00780493, HhCD00595131, HmCD00952869, SpCD00876354, and EdCD00959595, have the amino acid sequences set forth in SEQ ID Nos. 101-120, or encoded by the nucleotide sequences set forth in SEQ ID NOs. 41-60.

In certain embodiments, the subject is anti-p53 negative.

Another aspect of the disclosure comprises methods of diagnosing lung carcinoma in a subject, wherein Computer Tomography (CT) scanning of the subject identified an indeterminate pulmonary nodule, the method comprises: (i) providing a serum or blood sample from the subject; and (ii) contacting the serum or blood sample with an antibody panel described above. In some aspects, the method further comprises detecting the presence of an antibody against at least one antigen in the antibody panel in the serum or blood sample selected from the group consisting of an antigen having an amino acid sequence set forth in SEQ ID NOs. 65, 66, 69, 70, 72, 75, 76, 81, 86, 87, 91, 92, and 95-98 or encoded by SEQ ID NOs. 5, 6, 9, 10, 12, 15, 16, 21, 26, 27, 31, 32, and 35-38. Detection of an antibody against such an antigen in the subject's serum or blood sample indicates the indeterminate pulmonary nodule in the subject is lung carcinoma.

Some methods, the serum or blood sample is contacted with the antibody panel of one or more of the antibodies disclosed herein.

In some embodiments, the indeterminate pulmonary nodule in the subject is lung adenocarcinoma.

In certain embodiments, the method further comprising detecting the presence of an antibody against at least one antigen having an amino acid sequence set forth in SEQ ID NOs. SEQ ID NOs. 61-64, 67, 68, 71, 73, 74, 77-80, 103, 105, 106, 108, 109, 111, 113-117, 119, and 120 or encoded by SEQ ID NOs. 1-4, 7, 8, 11, 13, 14, 17-20, 43, 45, 46, 48, 49, 51, 53-57, 59, and 60. Detection of an antibody against such an antigen in the subject's serum or blood sample indicates the subject has a benign nodule.

In some embodiments, the serum or blood sample is a blood sample.

Yet another aspect concerns methods of reducing false positives in Computer Tomography (CT) screening of lung cancer, the method comprising: (i) providing a serum or blood sample from a subject, wherein CT screening of the subject identified an indeterminate pulmonary nodule; and
contacting the serum or blood sample with the antibody panel disclosed herein. In some embodiments, the method further comprises detecting the presence of an antibody against at least one antigen having an amino acid sequence set forth in SEQ ID NOs. 65, 66, 69, 70, 72, 75, 76, 81, 86, 87, 91, 92, and 95-98 or encoded by SEQ ID NOs. 5, 6, 9, 10, 12, 15, 16, 21, 26, 27, 31, 32, and 35-38. Detection of an antibody against such an antigen in the subject's serum or blood sample diagnoses the subject with lung carcinoma. In yet other embodiments, the method further comprises detecting the presence of an antibody against at least one antigen having an amino acid sequence set forth in SEQ ID NOs. 61-64, 67, 68, 71, 73, 74, 77-80, 103, 105, 106, 108, 109, 111, 113-117, and 119-120 or encoded by SEQ ID NOs. 1-4, 7, 8, 11, 13, 14, 17-20, 43, 45, 46, 48, 49, 51, 53-57, and 59-60. Detection of an antibody against such an antigen in the subject's serum or blood sample indicates the subject has a benign nodule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H depict, in accordance with certain embodiments, the fourth quartile odds ratio of anti-bacterial antibodies vs. P values for comparisons between ADC vs. BNC (FIG. 1A); ADC vs. BNC-E (FIG. 1i); ADC vs. BNC-G (FIG. 1 C); ADC vs. BNC-SN (FIG. 1 D); SMC vs. BNC (FIG. 1 E); SMC vs. BNC-E (FIG. 1 F); SMC vs. BNC-G (FIG. 1 G); and SMC vs. BNC-SN (FIG. 1 H). Dotted lines indicate p-value=0.05. Antibodies above the dotted lines had OR p-values <0.05. Antibodies against Helicobacter pylori, Pseudomonas aeruginosa and Streptococcus spp. antigens are show in triangle, square, and inverted triangle, respectively. The annotation “X>Y Z Abs” on each plot indicates that Z anti-bacterial antibodies with OR p-values <0.05 between X and Y groups had higher seroprevalence in X than in Y. ADC: Lung adenocarcinoma; BNC: Benign nodule controls; SMC: Smoker controls; BNC-E: Emphysema; BNC-G: Granuloma; BNC-SN: Stable nodule.

FIGS. 2A and 2B show, in accordance with certain embodiments, anti-H. pylori antibodies with differential prevalence between lung cancer patients and high-risk controls. FIG. 2A depicts fourth quartile odds ratio of anti-microbial antibodies between lung cancer patients and high-risk controls vs. P values. Antibodies against H. pylori and Streptococcus spp. are shown in triangle and inverted triangle respectively. The dotted line indicates p-value=0.05. FIG. 2B depicts dot plots for selected anti-H. Pylori antibodies. Anti-HP1341 and IgA had the highest reactivity among all anti-H. pylori antibodies. Anti-HP0596, Anti-HP0477, Anti-HP0923 are 3 examples of the anti-H. pylori antibodies showing significantly higher prevalence in lung adenocarcinoma patients (ADC) vs. smoker controls (SMC). Solid lines indicated seropositivity cutoff 2 of median normalized intensity.

FIGS. 3A-3C, in accordance with certain embodiments, compare anti-bacterial antibodies between anti-p53 positive and negative ADC patients. FIG. 3A is a heatmap showing antibodies with significantly higher prevalence in 101 anti-p53 negative ADC patients relative to 26 anti-p53 positive ADC patients. FIG. 3B is a dot plot for anti-p53 positive and negative ADC patients based on anti-p53 ELISA. The solid red line indicates the ELISA normalized O.D.450 seropositivity cutoff. FIG. 3C shows dot plots for anti-microbial antibodies against Haemophilus influenzae and Streptococcus spp. antigens between anti-p53 positive and negative ADC patients. Solid lines indicate seropositivity cutoff 2 of median normalized intensity.

FIGS. 4A-4C depict, in accordance with certain embodiments, Receiver Operating Characteristics (ROC) analysis for the antibody panels built using the logistic regression model with 20 antibodies selected using the MRMR method between ADC vs. BNC (FIG. 4A); ADC vs. SMC (FIG. 4B); and BNC vs. SMC (FIG. 4C).

FIGS. 5A and 5B depict, in accordance with certain embodiments, dot plots for the most reactive IgG and IgA antibodies for each bacterium and virus. For IgG, bacteria or viruses are sorted by the 75th percentile of the Median Normalized Intensity of all samples (FIG. 5A). For IgA, bacteria and viruses are plotted in the same order as IgG (FIG. 5B).

FIG. 6, in accordance with certain embodiments, shows the number of four types of antibodies among all 424 samples. The median number of anti-bacterial IgG, anti-bacterial IgA, anti-virus IgG, and anti-virus IgA antibodies were 77, 34, 103, and 19, respectively.

FIGS. 7A and 7B depict, in accordance with certain embodiments, Spearman rank correlation coefficients heatmaps for the most reactive IgG antibody (FIG. 7A) or most reactive IgA antibody (FIG. 7B) of each microorganism. Most reactive antibodies were determined by the median reactivity among all 434 samples for each IgG or IgA antibody. Antibodies are labeled by their target antigen UniProt ids and source microorganisms.

FIGS. 8A-8D depict, in accordance with certain embodiments, the association of clinical parameters with anti-bacterial antibodies. Fourth quartile Odds ratios of anti-microbial antibodies vs. p-values are plotted. Light smokers with <=20 pack-years smoking history vs. heavy smokers with >20 pack-years smoking history are shown in FIG. 8A. Fifty-three antibodies had p-values less than 0.05. Out of these, 51 had higher seroprevalence in light smokers and 2 in heavy smoker. BNC with small nodules <=1 cm vs. large nodules >1 cm are shown in FIG. 8B. Forty antibodies had p-values less than 0.05. Out of these, 0 had higher seroprevalence in small nodule BNC and 40 in large nodule BNC. Stage I ADC vs. Stage (II+III) ADC are shown in FIG. 8 C. Thirty-five antibodies had p-value less than 0.05. Out of these, 26 had higher prevalence in Stage I and 9 in Stage (II+III). Male vs. Female are shown in FIG. 8D. Forty-nine antibodies had p-values less than 0.05. Out of these, 37 had higher prevalence in males and 12 had higher prevalence in females.

FIGS. 9A-9F depict, in accordance with certain embodiment, ROC analysis for the 20-antibody panels built separately for each comparison using the logistic regression model between ADC vs. BNC-E (FIG. 9 A); ADC vs. BNC-G (FIG. 9B); ADC vs. BNC-SN (FIG. 9C); SMC vs. BNC-E (FIG. 9D); SMC vs. BNC-G (FIG. 9E); and SMC vs. BNC-SN (FIG. 9F). ADC: Lung adenocarcinoma; BNC: Benign nodule controls; SMC: Smoker controls; BNC-E: Emphysema; BNC-G: Granuloma; BNC-SN: Stable nodule.

### DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “false positive” refers a test result which incorrectly indicates that a particular condition or attribute is present. Accordingly, the “false positive indicator”, in some aspects, refers to a biomarker that indicates the corresponding positive test result incorrectly indicates the presence of a particular condition or attribute, for example, cancer or an autoimmune condition.

A polypeptide having an amino acid sequence selected from SEQ ID NOs. 61-120 is disclosed, wherein the isolated polypeptide may be used to screen for antibodies in a subject that categorizes the subject as a smoker, having a benign module, or having lung carcinoma (such as lung adenocarcinoma). In some aspects, a nucleic acid sequence selected from SEQ ID NO. 1-60 is disclosed, wherein the nucleic acid encodes a microbial antigen recognized by antibodies in a subject likely to be a smoker, have a benign module, or have lung carcinoma.

Accordingly, antibody panels for diagnosing if a subject has lung carcinoma and for distinguishing between malignant and benign indeterminate pulmonary nodules (IPN) detected by CT scan are also disclosed. These antibody panels were developed through exploring the microbial link in cancer. Though there is growing interest in a microbial link in the onset of lung cancer, there remains limited understanding on which and how certain microorganisms cause or contribute to lung cancer development.

Using the Nucleic Acid Programmable Protein Array (NAPPA), antibodies against about one thousand antigens from tens of different commensal and pathogenic bacteria and viruses were profiled to identify the antibody panels disclosed herein. Some of the different commensal and pathogenic bacteria and viruses have been reported to possess various degrees of potential association with lung cancer or respiratory diseases, though many have not previously been known to be associated with lung cancer. The antibody panels disclosed herein have an area under the curve (AUC) of 0.80 distinguishing lung adenocarcinoma (ADC) and benign nodule controls (BNC) with 43% sensitivity at 90% specificity and 46% specificity at 90% sensitivity. Accordingly, also disclosed are methods of identifying whether an IPN detected by CT scan in a subject is malignant or benign as well as methods of reducing false positive rate of lung cancer diagnosis using CT imaging.

Many causes of IPN relate to prior or concurrent microbial infection. These infections may lead to specific immune response signatures that can distinguish between malignant and benign IPNs. Previous studies suggest Chlamydia pneumoniae, human immunodeficiency virus (HIV), human papilloma virus (HPV), Streptococcus pneumoniae, and Mycobacterium tuberculosis may promote lung cancer development. This is an area of active research as previous studies were limited by small sample sizes, observational study designs and inconsistent results. Culture-free next-generation sequencing has improved understanding of compositional changes in various microbiota related to lung cancer. It is now clear that the lung has distinct microbiota that can promote or prevent disease. Besides lung microbiota, oral and gut microbiota have also been implicated in lung cancer development.

Microbial infection elicits complex host innate and adaptive immune responses. The humoral immune response represents an important aspect of the adaptive immune response. The detection of anti-microbial antibodies in blood, the products of the humoral immune response, has frequently informed disease research. Unlike sequencing analysis, which provides information on the presence or absence of microorganisms limited to the time of sampling, the elicitation of antibody against a microbial protein demonstrates a host response to the microorganism and allows us to infer an “infection history”. Studies of antibody response against microorganisms have relied on whole virus preparations or crude bacterial lysates of usual microbial suspects, one microorganism at a time. Challenges associated with these traditional methods include low throughput, inadequate quantification, and poor reproducibility. Moreover, they lack the ability to compare responses to multiple organisms in the same sample. Additionally, antibodies to specific microbial antigens are more informative than the overall infection status to detect cancer. Furthermore, the etiology for many epithelial cancers including lung cancer is most likely polymicrobial, involving cooperation of microorganisms that are non-pathogenic or weakly-pathogenic when acting alone, but able to initiate and promote tumorigenesis when acting together as a community.

Antibody response to bacteria or viruses, such as C. pneumoniae, Helicobacter pylori, HPV, and polyomaviruses, have been studied, but their associations with lung cancer remains elusive. Until the present disclosure, no systematic studies on the antibody responses against viral and bacterial proteins in lung cancer patients have been reported.

A history of previous lung disease such as chronic obstructive pulmonary disease (COPD), tuberculosis and pneumonia has been associated with an increased risk of developing lung cancer and/or the formation of IPNs. Common to all these lung diseases is a strong connection with microbial infections.

Just as ADC, BNC, and smoker controls (SMC) have distinct etiologies, the Examples show that they also have distinct anti-microbial antibody profiles. ADC patients generate antibodies to a subset of the thousands of antigens encoded by these microorganisms in a cancer-specific manner, whereas BNC generate antibodies to a different subset of the thousands of antigens encoded by these microorganisms in a control-specific manner, providing an opportunity to rule out cancer.

For antibodies showing significant ORs among the 3 groups, enrichment of source microorganisms for the target antigens with previously suspected or novel connections with lung diseases or lung cancer were observed. Besides disease diagnosis, the results from the Examples also revealed that cancer genetics, smoking history and nodule sizes affected anti-microbial antibody prevalence.

Most microorganisms had one or more antibodies showing high reactivity among most samples against their antigens displayed on the arrays. Interestingly, highly reactive antibodies or those with the highest prevalence for a microorganism generally did not show differential reactivity among different subject groups. On the contrary, antibodies with moderate reactivity/prevalence showed more differential reactivity among different subject groups. Similar results were observed for infections in other diseases. The ability of these specific antigen responses to distinguish subject groups speaks for the importance of assaying antibodies against individual proteins, especially proteins that are not the dominant antigens of the tested microbes. These results are consistent with previous finding that antibodies against antigens of the same microorganism displayed different trends of reactivity in healthy controls, gastric cancer (GC), and subjects with intestinal metaplasia. It is not immediately apparent why antigens from the same microorganism showed differential reactivity. The physiological conditions existing in the infected tissues of subjects in different health/disease states can modulate microbial antigen expression profiles, and the immunological microenvironment at the infection sites also affects humoral immune response to expressed antigens.

Besides exposure to smoke or other hazardous chemicals, microbial infection in the lung is the main cause of pulmonary diseases that can lead to the formation of nodules. Some of these nodules would remain benign, but some would progress into malignant nodules. Around one-fifth of the cancer are caused by microbes. Lungs are one of the organs which get exposed to the environmental microbes the most due to continuous breathing. Similar to smoking, microbial infection can cause chronic inflammation and mutation that can lead to cancer. The results in the Examples support the associations of bacterial, and to a lesser degree, viral infections with the development of benign pulmonary nodules. The data in the Examples showed overall higher prevalence of many anti-microbial antibodies in each of the 3 BNC subgroups compared with either ADC or SMC (Table 2). There can be several explanations why ADC patients had lower antibody prevalence relative to BNC. One possibility is that the microbial infections that caused benign nodules contribute less to cancer development. Alternatively, it is also possible that some of these microorganisms may contribute to benign nodules and cancer initiation but play a lesser role in the progression and maintenance of cancer. It is interesting to note the granuloma, whose etiology has the strongest known association with microbial infection, showed strongest anti-microbial antibody reactivity among 3 subgroups of BNC.

Antibodies against H. pylori, a known carcinogenic microbe, showed greatest overall trend differences among ADC, SMC, and BNC. H. pylori is one of the best studied bacteria because of its etiological role in many digestives and extra-digestive diseases, especially in GC. H. pylori has also been associated with many respiratory disorders, including COPD, bronchiectasis, asthma, tuberculosis, and lung cancer. However, results from previous sero-epidemiological studies were inconsistent. The data in the Examples suggest a role of H. pylori infection in ADC. Two possible carcinogenic mechanisms have been proposed for how H. pylori infection might contribute to lung cancer development. First, the lungs originate embryologically from the same endoderm cells which form the epithelia lining of the digestive tract, where gastrin provides the major proliferative stimulus. H. pylori infection in the stomach results in the enhanced and prolonged release of gastrin in circulation, which also stimulates the proliferation of bronchial epithelium. In addition, immune effector proteins such as cytokines produced from native or adaptive immune response to H. pylori infection in the stomach may also enter the circulation causing a stimulatory effect on lung cell proliferation. Second, H. pylori infection in the lung could cause direct damage and chronic airway inflammation that promote lung cancer development. However, H. pylori has not yet been detected in human bronchial tissue or isolated from bronchoalveolar lavage fluid. Furthermore, even for GC, only an extremely small percentage of people infected with H. pylori develop GC. It is believed that co-infection with additional microorganisms, besides genetic and other environmental factors, contributes to cancer development. However, for the set of microbial antigens displayed on the tested arrays, none of their antibodies had significant positive or negative correlation with anti-H. pylori antibodies. Future studies with more antigens and organisms may shed light on the polymicrobial nature of the pathogenesis of H. pylori infection in lung cancer.

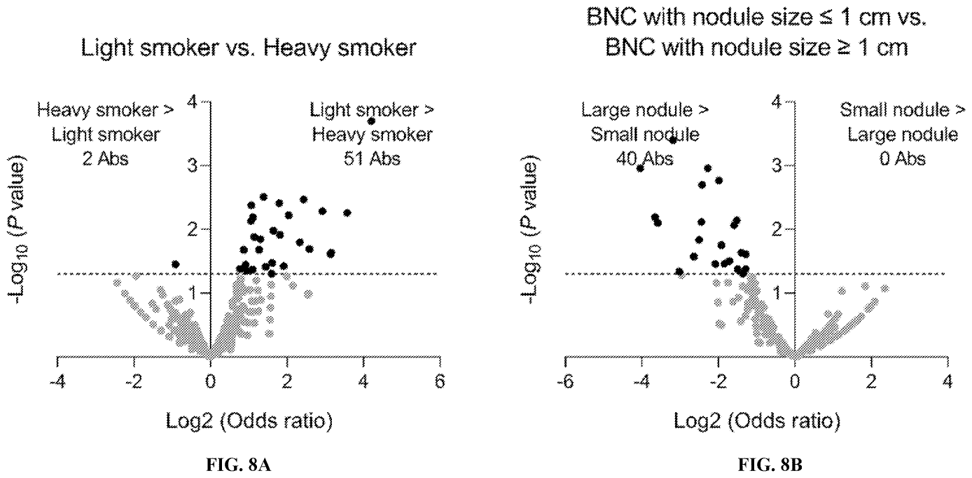

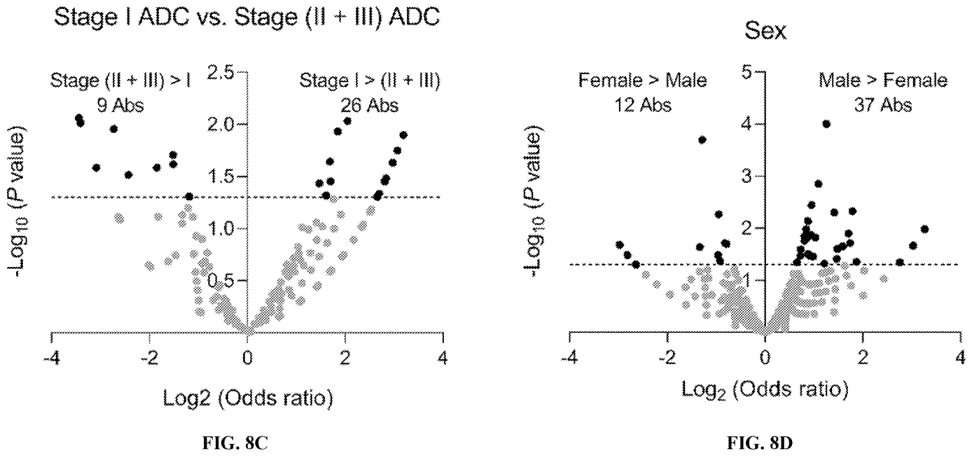

Many factors including disease stages, disease grades, genetic background, microbial antigen expression levels, local and global immune environment contribute to the heterogeneity in antibody response. Among them, smoking is a major contributing factor. The results in the Examples suggest that the role microbial infection plays and the level of its importance in lung disease pathology might be different in light and heavy smokers (FIG. 8A). It is plausible that microbial infection might play a more important role in disease etiology in light smokers than in heavy smokers. Alternatively, smoking may impair lung immunity and dampen antibody response to microbial infection to a greater extent in heavy smokers than in light smokers. Similar to smoking habits, early disease stage (stage I ADC) led to higher antibody prevalence compared to later disease stage (stage II, III ADC). This observation may be due to the reduction in immunity caused by the progression of disease.

The same set of samples was used study tumor associated autoantibodies (TAAb) and anti-microbial antibodies. No significant correlation between autoantibodies and anti-microbial antibodies was observed. TAAb against p53 is probably the most studied autoantibody in cancer. One prerequisite for eliciting anti-p53 is the over-expression of p53 due to its mutation. The presence or absence of a p53 mutation leads to different disease etiologies. For example, EBV-associated GC had lower prevalence of anti-p53 antibody relative to GC not associated with EBV. The p53 mutation status for the study population was not known. Using anti-p53 seropositivity as a surrogate (i.e., there was a high likelihood of p53 mutation for anti-p53 positive individuals), anti-Streptococcus spp. antibodies have strikingly higher prevalence in anti-p53 negative cases than anti-p53 positive cases. This suggests that Streptococcus spp. and p53 mutation may play mutually exclusive roles in lung cancer development.

As shown in the Examples, the effect of the microbiome on the host immune response in lung or at distal sites is important for lung cancer initiation and progression. Anti-microbial antibodies reveal one important aspect of host adaptive immune response to microbial infection and provides a starting point for further investigation of the link between the microorganism and cancer.

### Antibody Panel

The antibody panel described herein related to methods of diagnosing lung carcinoma comprises at least one antigen from at least one bacterium selected from the group consisting of: Helicobacter pylori, Pseudomonas aeruginosa, Streptococcus pneumoniae, Haemophilus influenzae, Streptococcus pyogenes, Mycobacterium tuberculosis, Streptococcus gallolyticus, Fusobacterium nucleatum, Klebsiella pneumoniae, Escherichia coli, Gemella haemolysans, Bacteroides vulgatus, Campylobacter jejuni, Bacteroides fragilis, Peptostreptococcus anaerobius, Citrobacter koseri, Leptotrichia buccalis, Clostridium difficile, Enterococcus faecalis, Porphyromonas gingivalis, Bifidobacterium adolescentis, Prevotella copri, Faecalibacterium prausnitzii, and Veillonella parvula. In some embodiments, the antibody panel further comprises at least one antigen from at least one virus selected from the group consisting of: human herpesvirus, human mastadenovirus, influenza A virus, human coronavirus OC43, enterovirus, rhinovirus B, human parainfluenza virus 1, human metapneumovirus, human respiratory syncytial virus B, and human coronavirus 229E. In some aspects, the at least one virus is selected from the group consisting of: human herpesvirus 4, human mastadenovirus C, influenza A virus, human herpesvirus 5, human mastadenovirus F, human coronavirus OC43, enterovirus C, human mastadenovirus B, rhinovirus B, human parainfluenza virus 1, human metapneumovirus, human respiratory syncytial virus B, human coronavirus 229E, and enterovirus D.

In some embodiments, the antibody panel comprises at least one antigen from at least one bacterium selected from the group consisting of: C. jejuni, E. faecalis, H. pylori, P. aeruginosa, S. pneumoniae, H. influenzae, S. pyogenes, M tuberculosis, S. gallolyticus, and F. nucleatum. In some embodiments, the antibody panel comprises at least one antigen from at least one virus selected from the group consisting of: human herpesvirus 4, human mastadenovirus C, influenza A virus, human herpesvirus 5, human mastadenovirus F, and human coronavirus OC43.

In particular embodiments, the antibody panel comprises at least one antigen from at least one microorganism selected from the group consisting of: C. jejuni, G. haemolysans, H. pylori, human herpesvirus, influenza A virus, P. aeruginosa, S. gallolyticus, S. pneumoniae, and S. pyogenes. In some implementation, the antibody panel comprises at least one antigen from C. jejuni, G. haemolysans, H. pylori, human herpesvirus 4, influenza A virus (H1N1), P. aeruginosa, S. gallolyticus, S. pneumoniae, and S. pyogenes.

In particular embodiments, the antibody panel comprises at least one antigen from at least one microorganism selected from the group consisting of: adenovirus, H. pylori, human herpesvirus, human mastadenovirus, human parainfluenza virus, human rhinovirus, M. tuberculosis, P. aeruginosa, S. gallolyticus, S. pneumoniae, and S. pyogenes. In some implementation, the antibody panel comprises at least one antigen from human adenovirus 41, H. pylori, human herpesvirus 4, human herpesvirus 5, human mastadenovirus A, human parainfluenza virus 1, human rhinovirus A1, M. tuberculosis, P. aeruginosa, S. gallolyticus, S. pneumoniae, and S. pyogenes.

In particular embodiments, the antibody panel comprises at least one antigen from at least one microorganism selected from the group consisting of: enterovirus, influenza virus, H. pylori, human herpesvirus, human mastadenovirus, human metapneumovirus, human parainfluenza virus, human rhinovirus, M. tuberculosis, P. aeruginosa, S. pneumoniae, and S. pyogenes. In some implementation, the antibody panel comprises at least one antigen from enterovirus D68, influenza A virus (H3N2), influenza C virus, H. pylori, human herpesvirus 4, human mastadenovirus C strain, human mastadenovirus D strain, human metapneumovirus, human parainfluenza virus 4A, human rhinovirus A1, M tuberculosis, P. aeruginosa, S. pneumoniae, and S. pyogenes.

In certain embodiments, the antibody panel comprises at least 20 antigens detected by the antibodies listed in Table 3. In some aspects, the antibody panel comprises at least 20 antigens encoded by the nucleotide sequences set forth in SEQ ID NOs. 1-60 or having an amino acid sequence set forth in SEQ ID NOs. 61-120.

For example, in certain embodiments, the antibody panel comprises antigens detected by the antibodies SgCD00783211, PaCD00632112, HpCD00781518, HhCD00595176, HpCD00780533, HpCD00781139, GhCD00811667, SpCD00810996, SpCD00876258, PaCD00812634, SpCD00810910, HpCD00781182, IaCD00844155, HpCD00781050, HsCD00959752, HpCD00780821, HpCD00780927, CjCD00811525, SpCD00876160, and HhCD00595190 or encoded by the nucleotide sequences set forth in SEQ ID NOs. 1-20. In some aspects, the antibody panel comprises antigens having an amino acid sequence set forth in SEQ ID Nos. 61-80. In such embodiments, the antibody panel is useful for distinguishing between subjects with lung carcinoma and subjects with a benign lung tumor.

As another example, the antibody panel comprises antigens detected by the antibodies SpCD00818023, SgCD00783211, VzCD00594955, HhCD00594867, PaCD00632112, HrCD00959476, HaCD00953227, SgCD00811573, HhCD00595176, SpCD00818032, HpCD00780240, HpCD00780836, HmCD00952743, HmCD00849349, MtCD00544325, SpCD00875948, HpCD00781213, HpCD00849291, HhCD00595168, and SpCD00810863 or encoded by the nucleotide sequences set forth in SEQ ID NOs. 21-40. In some aspects, the antibody panel comprises antigens having an amino acid sequence set forth in SEQ ID Nos. 81-100. In such embodiments, the antibody panel is useful for distinguishing between subjects with lung carcinoma (for example, lung adenocarcinoma) and subjects with a history of smoking.

As yet another example, the antibody panel comprises antigens detected by the antibodies HsCD00959791, HmCD00849346, PaCD00632105, HmCD00849349, SpCD00818023, IcCD00953076, HmCD00956407, HpCD00780836, MtCD00412192, HmCD00849278, SpCD00876414, HpCD00781182, HrCD00959475, HpCD00781725, HpCD00953108, HpCD00780493, HhCD00595131, HmCD00952869, SpCD00876354, and EdCD00959595 or encoded by the nucleotide sequences set forth in SEQ ID NOs. 41-60. In some aspects, the antibody panel comprises antigens having an amino acid sequence set forth in SEQ ID Nos. 101-120. In such embodiments, the antibody panel is useful for distinguishing between subjects with a benign lung tumor and subjects with a history of smoking.

### Methods of Use

Also disclosed herein are methods of diagnosing lung carcinoma, for example lung adenocarcinoma.

In some embodiments, the method comprises providing a serum or blood sample from the subject; and detecting the presence of antibodies in the serum or blood sample that recognizes at least one H. pylori antigens selected from the group consisting of: HP0492, HP0305, tsf, HP0528, HP1564, HP1379, HP0406, HP0373, HP0923, HP0373, HP0528, HP1488, and oorA. In some aspects, the at least one H. pylori antigens has an amino acid sequence selected from the group consisting of SEQ ID NOs. 63, 65, 66, 72, 74, 76, 77, 91, 92, 97, 108, 112, and 114-116. In other aspects, the at least one H. pylori antigens is encoded by a nucleotide sequence selected from the group consisting of SEQ ID NOs. 3, 5, 6, 12, 14, 16, 17, 31, 32, 37, 48, 52, 54, and 56. Detecting the presence of the antibodies that recognizes at least one H. pylori antigen having an amino acid set forth in SEQ ID NOs. 65, 66, 72, 76, 91, 92, 97, 98 in the serum or blood sample indicates the subject has lung carcinoma, such as lung adenocarcinoma. Detecting the presence of the antibodies that recognizes at least one H. pylori antigen having an amino acid set forth in SEQ ID NOs. 63, 74, 77, 108, 114-115 in the serum or blood sample indicates the subject has benign nodule. Detecting the presence of the antibodies that recognizes at least one H. pylori antigen having an amino acid of SEQ ID NO. 112 in the serum or blood sample indicates the subject is a smoker. In some aspects, the antibodies detected are IgA. In some aspects, the antibodies detected are IgG. In certain implementations, the antibodies detected are IgA and IgG.

A method of diagnosing lung carcinoma in a subject, wherein CT scanning of the subject identified an indeterminate pulmonary nodule is also described. The method comprises providing a serum or blood sample from the subject; and contacting the serum or blood sample with an antibody panel disclosed herein. In some aspects, the antibody panel comprises at least one antigen selected from the group consisting of an antigen encoded by SEQ ID NOs. 5, 6, 9, 10, 12, 15, 16, 21, 26, 27, 31, 32, and 35-38 or selected from the group consisting of an antigen having the amino acid sequence set forth in SEQ ID NOs. 65, 66, 69, 70, 72, 75, 76, 81, 86, 87, 91, 92, and 95-98. Detecting the presence of an antibody against at least one antigen encoded by SEQ ID NOs. 5, 6, 9, 10, 12, 15, 16, 21, 26, 27, 31, 32, or 35-38 or having the amino acid sequence set forth in SEQ ID NOs. 65, 66, 69, 70, 72, 75, 76, 81, 86, 87, 91, 92, and 95-98 indicates the indeterminate pulmonary nodule in the subject is lung carcinoma. In some implementations, detecting the presence of an antibody against at least one antigen encoded by SEQ ID NOs. 5, 6, 9, 10, 12, 15, 16, 21, 26, 27, 31, 32, or 35-38 the amino acid sequence set forth in SEQ ID NOs. 65, 66, 69, 70, 72, 75, 76, 81, 86, 87, 91, 92, and 95-98 indicates the indeterminate pulmonary nodule in the subject is lung adenocarcinoma. In some implementations, the antibody panel comprises at least one antigen from at least one bacterium selected from the group consisting of: C. jejuni, E. faecalis, H. pylori, P. aeruginosa, S. pneumoniae, H. influenzae, S. pyogenes, M tuberculosis, S. gallolyticus, and F. nucleatum. In some embodiments, the antibody panel comprises at least one antigen from at least one virus selected from the group consisting of: human herpesvirus 4, human mastadenovirus C, influenza A virus, human herpesvirus 5, human mastadenovirus F, and Human coronavirus OC43. In other implementations, the antibody panel comprises at least one antigen from at least one microorganism selected from the group consisting of: C. jejuni, G. haemolysans, H. pylori, human herpesvirus, influenza A virus, P. aeruginosa, S. gallolyticus, S. pneumoniae, and S. pyogenes. In some implementation, the antibody panel comprises at least one antigen from C. jejuni, G. haemolysans, H. pylori, human herpesvirus 4, influenza A virus (H1N1), P. aeruginosa, S. gallolyticus, S. pneumoniae, and S. pyogenes.

In some aspects, the antibody panel comprises at least one antigen selected from the group having the amino acid sequence set forth in SEQ ID NOs. 61-64, 67, 68, 71, 73, 74, 77, 78, 79, 80, 82-85, 88-90, 93, 94, 99, and 100 or selected from the group consisting of an antigen encoded by SEQ ID NOs. 1-4, 7, 8, 11, 13, 14, 17, 18-25, 28-30, 33, 34, 39, and 40. Detecting the presence of an antibody against at least one such antigen indicates an increased likelihood the subject does not have lung carcinoma. For example, the subject instead has a benign nodule, especially when the presence of an antibody against at least one antigen having the amino acid sequence set forth in SEQ ID NOs. 61-64, 67, 68, 71, 73, 74, 77, 78, 79, or 80 is detected.

Further described herein is a method of reducing false positives in CT screening of lung cancer. The method comprises providing a serum or blood sample from a subject, wherein CT screening of the subject identified an indeterminate pulmonary nodule; and contacting the serum or blood sample with an antibody panel disclosed herein. In some aspects, the antibody panel comprises at least one antigen selected from the group consisting of an antigen encoded by SEQ ID NOs. 1-60 or an antigen having an amino acid sequence set forth in SEQ ID NO. 61-120. Detecting the presence of an antibody against at least one antigen encoded by SEQ ID NOs. 5, 6, 9, 10, 12, 15, 16, 21, 26, 27, 31, 32, and 35-38 or having an amino acid sequence set forth in SEQ ID NOs. 65, 66, 69, 70, 72, 75, 76, 81, 86, 87, 91, 92, and 95-98 in the serum or blood sample diagnoses the subject with lung carcinoma. Detecting the presence of an antibody against at least one antigen encoded by SEQ ID NOs. 1-4, 7, 8, 11, 13, 14, 17-20, 43, 45, 46, 48, 49, 51, 53-57, and 59-60 or having an amino acid sequence set forth in SEQ ID NOs. 61-64, 67, 68, 71, 73, 74, 77-80, 103, 105, 106, 108, 109, 111, 113-117, and 119-120 in the serum or blood sample indicates the subject has a benign nodule. In some implementations, detecting the presence of an antibody against at least one antigen encoded by SEQ ID NOs. 5, 6, 9, 10, 12, 15, 16, 21, 26, 27, 31, 32, or 35-38 or having an amino acid sequence set forth in SEQ ID NOs. 65, 66, 69, 70, 72, 75, 76, 81, 86, 87, 91, 92, or 95-98 in the serum or blood sample indicates the indeterminate pulmonary nodule in the subject is lung adenocarcinoma. In some implementations, the antibody panel comprises at least one antigen from at least one bacterium selected from the group consisting of: C. jejuni, E. faecalis, H. pylori, P. aeruginosa, S. pneumoniae, H. influenzae, S. pyogenes, M tuberculosis, S. gallolyticus, and F. nucleatum. In some embodiments, the antibody panel comprises at least one antigen from at least one virus selected from the group consisting of: human herpesvirus 4, human mastadenovirus C, influenza A virus, human herpesvirus 5, human mastadenovirus F, and Human coronavirus OC43. In other implementations, the antibody panel comprises at least one antigen from at least one microorganism selected from the group consisting of: C. jejuni, G. haemolysans, H. pylori, human herpesvirus, influenza A virus, P. aeruginosa, S. gallolyticus, S. pneumoniae, and S. pyogenes. In some implementation, the antibody panel comprises at least one antigen from C. jejuni, G. haemolysans, H. pylori, human herpesvirus 4, influenza A virus (H1N1), P. aeruginosa, S. gallolyticus, S. pneumoniae, and S. pyogenes. In still other implementations, the antibody panel comprises at least one antigen from at least one microorganism selected from the group consisting of: adenovirus, H. pylori, human herpesvirus, human mastadenovirus, human parainfluenza virus, human rhinovirus, M. tuberculosis, P. aeruginosa, S. gallolyticus, S. pneumoniae, and S. pyogenes. In some implementation, the antibody panel comprises at least one antigen from human adenovirus 41, H. pylori, human herpesvirus 4, human herpesvirus 5, human mastadenovirus A, human parainfluenza virus 1, human rhinovirus A1, M tuberculosis, P. aeruginosa, S. gallolyticus, S. pneumoniae, and S. pyogenes.

In some aspects, diagnostic uses for an isolated polypeptide having an amino acid sequence with at least 90% sequence identity to an amino acid sequence set forth in SEQ ID NOs. 61-120 or isolated nucleic acid sequence having at least 80% sequence identity to a sequence selected from SEQ ID NOs. 1-60 and encodes an amino acid sequence set forth in SEQ ID NOs. 61-120 are disclosed. The use of the aforementioned isolated polypeptide or isolated nucleic acid sequence for in the manufacture of a diagnostic panel. In another embodiment, use of the use of the aforementioned isolated polypeptide or isolated nucleic acid sequence in the diagnosis of lung cancer is disclosed, for example, a use in diagnosing lung carcinoma in a subject.

### EXAMPLES

The invention is further illustrated by the following examples that should not be construed as limiting. The contents of all references, patents, and published patent applications cited throughout this application, as well as the Figures, are incorporated herein by reference in their entirety for all purposes.

### I. Anti-Microbial Antibody Profiling on Microbial Antigen Arrays

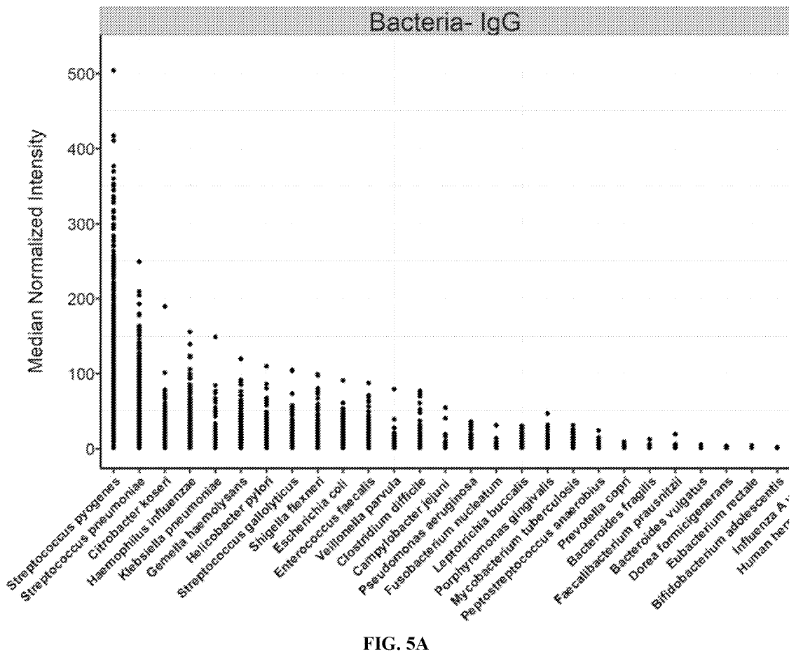

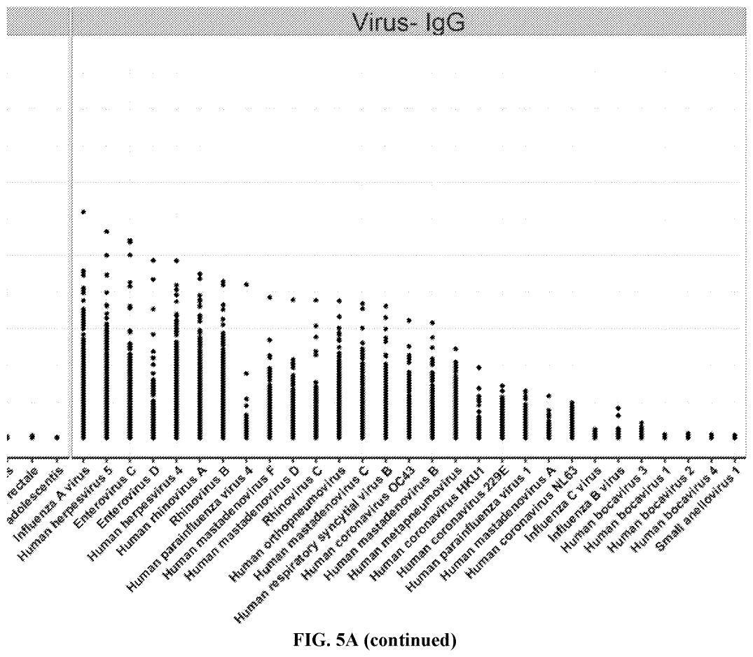

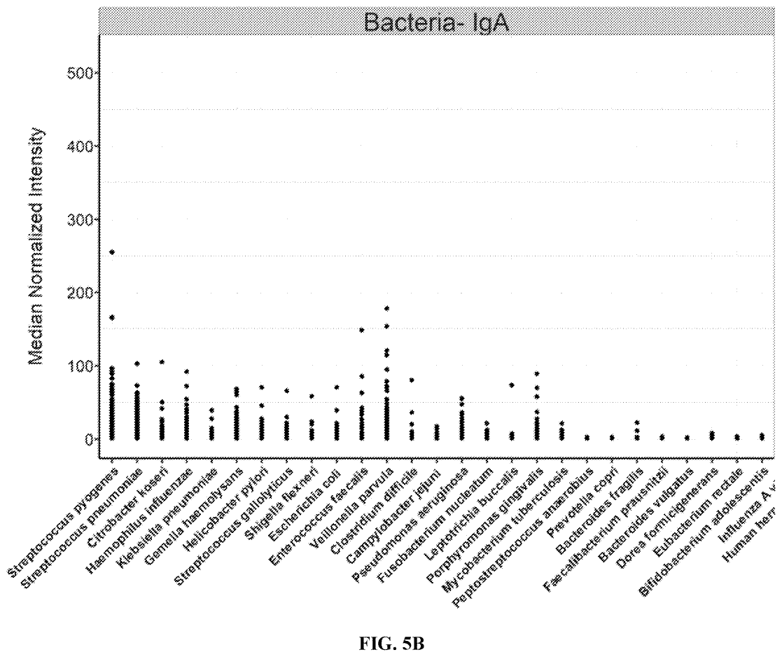

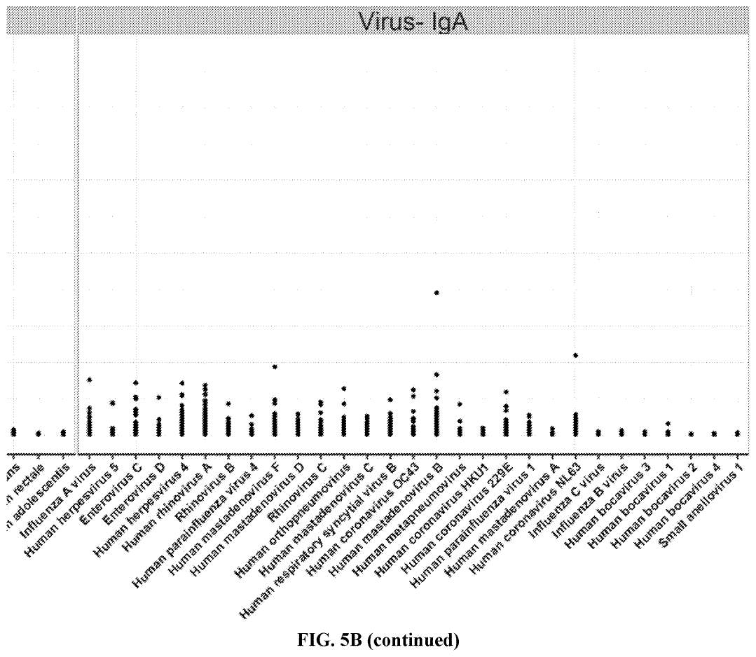

Plasma samples were profiled to identify anti-microbial antibodies against antigens from a set of representative microorganisms in 420 subjects, including ADC, BNC, and SMC (Table 1). Because of the important role of IgA in lung immunity, both IgG and IgA anti-microbial antibodies were profiled. Most microorganisms included in this study had one or more antigens showing antibody response in more than 10% of the study population. Streptococcus pyogenes, Streptococcus pneumoniae, and Haemophilus influenzae elicited the strongest IgG antibody response among bacteria, while respiratory viruses, such as rhinoviruses, coronaviruses, influenza viruses and adenoviruses elicited the strongest IgG antibody response among viruses (FIG. 5A).

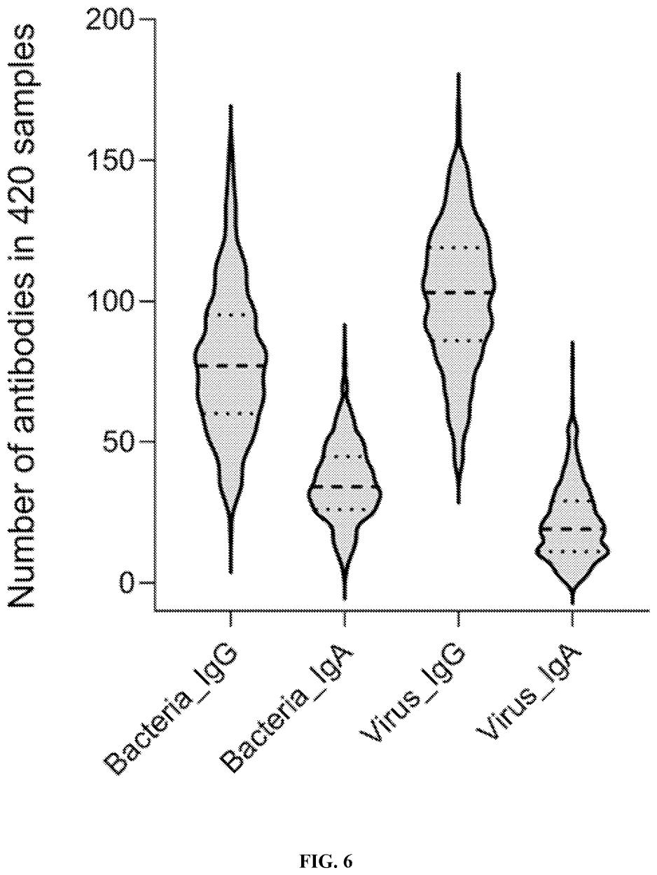

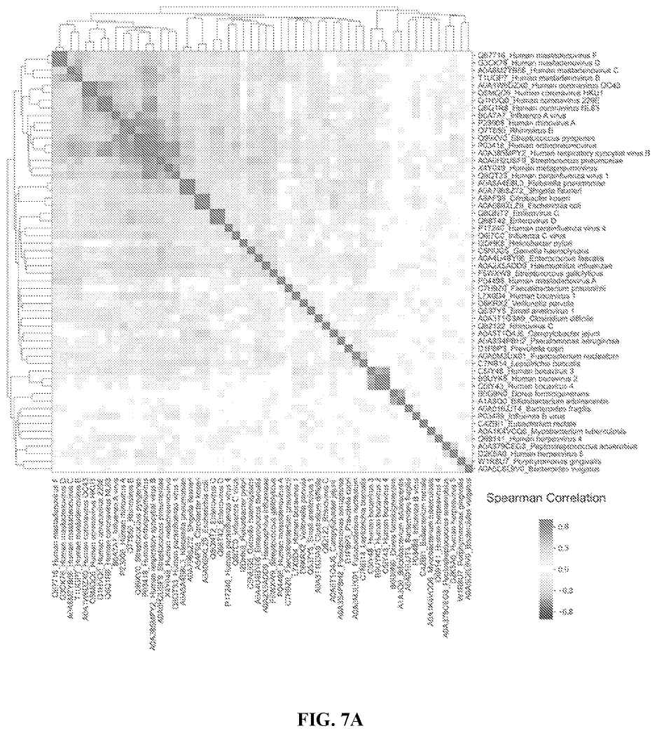

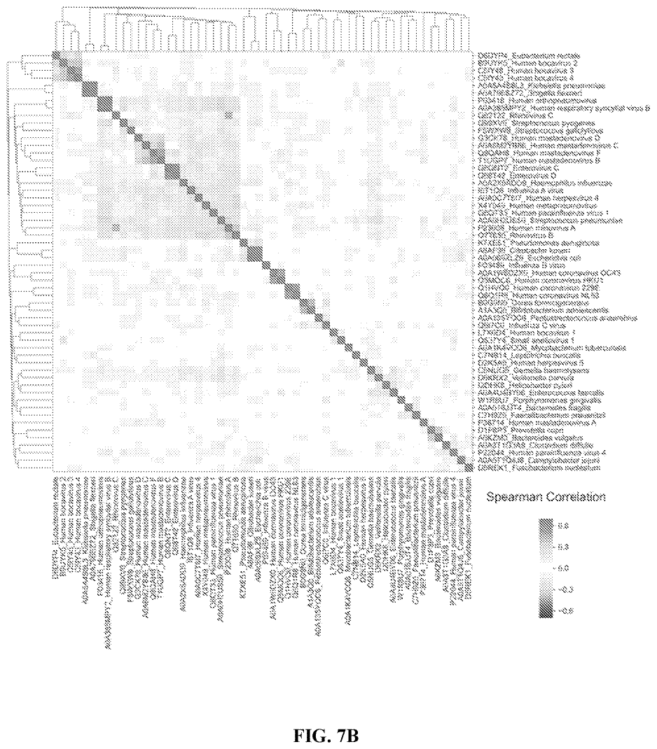

Microorganisms with higher IgG antibody reactivity also had higher IgA antibody reactivity, although IgA reactivity was usually lower than IgG for the same antigen (FIGS. 5A and 5B). The median numbers of anti-bacterial IgG and IgA antibodies were 77 and 34 out of 579 possibilities, respectively (FIG. 6). The medians for anti-viral IgG and IgA antibodies were 103 and 19 out of 336 possibilities, respectively (FIG. 6). The analysis was focused on anti-bacterial antibodies because anti-viral antibodies showed similar reactivity with no clear trend of differences among ADC, BNC, and SMC (Tables 2, 3, and 4). The correlation of the most reactive antibody of each microorganism among all subjects was analyzed (FIGS. 7A and 7B). Positive correlations between closely related bacteria and viruses for both IgG and IgA antibodies, such as Shigella flexneri and Klebsiella pneumoniae, and among the 4 seasonal human coronaviruses OC43, HKU1, 229E, and NL63 were observed. Correlations among microorganisms of different families or between viruses and bacteria were weak.

TABLE 1
Demographic information of 127 lung adenocarcinoma patients,
123 smoker controls and 170 benign nodule controls.

| | | | Benign nodule control | | |
|---|---|---|---|---|---|
| | Lung | Smoker | | | Stable |
| Characteristics | adenocarcinoma | controls | Emphysema | Granuloma | nodule |
| N | 127 | 123 | 47 | 50 | 73 |
| Mean age | 70 ± 10 | 64 ± 11 | 68 ± 8 | 63 ± 12 | 62 ± 10 |
| Sex | | | | | |
| Male | 54 | 52 | 21 | 25 | 28 |
| Female | 73 | 70 | 25 | 25 | 45 |
| No data | | 1 | 1 | | |
| Smoking | | | | | |
| history | | | | | |
| Never | 11 | 0 | 1 | 8 | 2 |
| Former | 82 | 91 | 30 | 28 | 44 |
| Current | 20 | 31 | 15 | 8 | 26 |
| No data | 14 | 1 | 1 | 6 | 1 |
| Mean pack | 31 ± 27 | 33 ± 24 | 50 ± 32 | 28 ± 47 | 30 ± 19 |
| years | | | | | |
| Nodules Size, | 2.3 ± 1.4 | 0 | 0.4 ± 0.3 | 1.9 ± 1.6 | 0.6 ± 0.3 |
| cm | | | | | |
| Stage | | | | | |
| I | 84 | | | | |
| II | 29 | | | | |
| III | 14 | | | | |

TABLE 2
Prevalence of anti-bacterial antibodies with odds ratio p-values <
0.05 in two comparison groups and their significance of differences.

| | | | | Two | |
|---|---|---|---|---|---|
| | | Number of | Number of | sample | Number |
| | | antibodies with | antibodies with | proportion | of |
| Groups | | higher | higher | test P | different |
| A | B | prevalence in A | prevalence in B | value | species |
| ADC | BNC | 8 | 23 | <0.001 | 9 |
| ADC | BNC-E | 6 | 28 | <0.001 | 12 |
| ADC | BNC-G | 0 | 36 | <0.001 | 12 |
| ADC | BNC-SN | 2 | 29 | <0.001 | 13 |
| SMC | BNC | 3 | 23 | <0.001 | 7 |
| SMC | BNC-E | 4 | 39 | <0.001 | 9 |
| SMC | BNC-G | 1 | 45 | <0.001 | 10 |
| SMC | BNC-SN | 4 | 29 | <0.001 | 8 |
| Light | Heavy | 51 | 2 | <0.001 | 12 |
| smokers | smokers | | | | |
| with <=20 | with >20 | | | | |
| pack-years | pack-years | | | | |
| smoking | smoking | | | | |
| history | history | | | | |
| Benign | Benign | 0 | 40 | <0.001 | 14 |
| nodule | nodule | | | | |
| size <= 1 cm | size > 1 cm | | | | |
| Stage I ADC | Stage II and | 26 | 9 | <0.001 | 12 |
| | III ADC | | | | |
| Male | Female | 37 | 12 | <0.001 | 17 |

TABLE 3
The antibodies marker and their respective coefficient used for the ROC curves.

| ADC vs. BNC | | | ADC vs. SMC | | | SMC vs. BNC | | |
|---|---|---|---|---|---|---|---|---|
| DNASU id | Isotype | Coefficient | DNASU id | Isotype | Coefficient | DNASU id | Isotype | Coefficient |
| SgCD00783211 | IgG | −0.53 | SpCD00818023 | IgA | 0.06 | HsCD00959791 | IgG | 1.46 |
| PaCD00632112 | IgG | −0.35 | SgCD00783211 | IgG | −0.69 | HmCD00849346 | IgG | 21.56 |
| HpCD00781518 | IgG | −0.35 | VzCD00594955 | IgG | −0.64 | PaCD00632105 | IgG | −0.15 |
| HhCD00595176 | IgG | −0.08 | HhCD00594867 | IgA | −1.83 | HmCD00849349 | IgG | 0.33 |
| HpCD00780533 | IgA | 1.37 | PaCD00632112 | IgG | −0.3 | SpCD00818023 | IgA | −0.02 |
| HpCD00781139 | IgA | 2.47 | HrCD00959476 | IgG | 0.02 | IcCD00953076 | IgG | −0.12 |
| GhCD00811667 | IgG | −1.25 | HaCD00953227 | IgA | 0.64 | HmCD00956407 | IgG | 0.02 |
| SpCD00810996 | IgG | −1.2 | SgCD00811573 | IgG | −0.42 | HpCD00780836 | IgA | −19.89 |
| SpCD00876258 | IgA | 0.02 | HhCD00595176 | IgG | −0.07 | MtCD00412192 | IgG | −1.39 |
| PaCD00812634 | IgG | 1.48 | SpCD00818032 | IgG | −0.12 | HmCD00849278 | IgG | 0.11 |
| SpCD00810910 | IgG | −0.07 | HpCD00780240 | IgA | 1 | SpCD00876414 | IgG | −0.03 |
| HpCD00781182 | IgG | 0.36 | HpCD00780836 | IgA | 17.79 | HpCD00781182 | IgG | 0.31 |
| IaCD00844155 | IgG | −0.21 | HmCD00952743 | IgG | −0.12 | HrCD00959475 | IgA | −0.62 |
| HpCD00781050 | IgA | −0.32 | HmCD00849349 | IgG | −0.41 | HpCD00781725 | IgG | −0.09 |
| HsCD00959752 | IgG | 0.09 | MtCD00544325 | IgG | 2.45 | HpCD00953108 | IgG | −1.27 |
| HpCD00780821 | IgG | 1.08 | SpCD00875948 | IgA | 0.02 | HpCD00780493 | IgG | −0.61 |
| HpCD00780927 | IgG | −0.02 | HpCD00781213 | IgA | 0.12 | HhCD00595131 | IgG | −0.08 |
| CjCD00811525 | IgG | −0.05 | HpCD00849291 | IgA | 0.02 | HmCD00952869 | IgA | 0.31 |
| SpCD00876160 | IgG | −0.01 | HhCD00595168 | IgG | −0.19 | SpCD00876354 | IgG | −0.2 |
| HhCD00595190 | IgG | −0.02 | SpCD00810863 | IgG | −0.93 | EdCD00959595 | IgA | −0.26 |

Twenty antibodies and their coefficient values were determined using Maximum Relevance − Minimum Redundance (MRMR) logistic regression modelling.

TABLE 4

| | | | | SEQ ID | |
|---|---|---|---|---|---|
| | | | | NO. | |
| DNASU id | Isotype | Antigen | Species | DNA | AA |
| SgCD00783211 | IgG | | Streptococcus gallolyticus subsp. | 1 | 61 |
| | | | gallolyticus ATCC 43143 | | |
| PaCD00632112 | IgG | PA0833 | Pseudomonas aeruginosa | 2 | 62 |
| HpCD00781518 | IgG | HP0492 | Helicobacter pylori 26695 | 3 | 63 |
| HhCD00595176 | IgG | BVRF1 | Human herpesvirus 4 | 4 | 64 |
| HpCD00780533 | IgA | HP0305 | Helicobacter pylori 26695 | 5 | 65 |
| HpCD00781139 | IgA | tsf | Helicobacter pylori 26695 | 6 | 66 |
| GhCD00811667 | IgG | | Gemella haemolysans ATCC | 7 | 67 |
| | | | 10379 | | |
| SpCD00810996 | IgG | SP_1000 | Streptococcus pneumoniae | 8 | 68 |
| | | | TIGR4 | | |
| SpCD00876258 | IgA | emm1 | Streptococcus pyogenes M1 GAS | 9 | 69 |
| PaCD00812634 | IgG | pscJ | Pseudomonas aeruginosa | 10 | 70 |
| SpCD00810910 | IgG | SP_1923 | Streptococcus pneumoniae | 11 | 71 |
| | | | TIGR4 | | |
| HpCD00781182 | IgG | HP0528 | Helicobacter pylori 26695 | 12 | 72 |
| IaCD00844155 | IgG | HA | Influenza A virus (A/Puerto | 13 | 73 |
| | | | Rico/8/34/Mount Sinai(H1N1)) | | |
| HpCD00781050 | IgA | HP1564 | Helicobacter pylori 26695 | 14 | 74 |
| HsCD00959752 | IgG | NS1 | H1N1 subtype | 15 | 75 |
| HpCD00780821 | IgG | | Helicobacter pylori 26695 | 16 | 76 |
| HpCD00780927 | IgG | HP1379 | Helicobacter pylori 26695 | 17 | 77 |
| CjCD00811525 | IgG | | Campylobacter jejuni subsp. | 18 | 78 |
| | | | jejuni NCTC 11168 | | |
| SpCD00876160 | IgG | SPy_1558 | Streptococcus pyogenes M1 GAS | 19 | 79 |
| HhCD00595190 | IgG | BSLF2/BM | Human herpesvirus 4 | 20 | 80 |
| SpCD00818023 | IgA | SP_2190 | Streptococcus pneumoniae | 21 | 81 |
| | | | TIGR4 | | |
| SgCD00783211 | IgG | | Streptococcus gallolyticus subsp. | 22 | 82 |
| | | | gallolyticus ATCC 43143 | | |
| VzCD00594955 | IgG | | Adenovirus | 23 | 83 |
| HhCD00594867 | IgA | UL122 | Human herpesvirus 5 | 24 | 84 |
| PaCD00632112 | IgG | PA0833 | Pseudomonas aeruginosa | 25 | 85 |
| HrCD00959476 | IgG | | Human rhinovirus A1 | 26 | 86 |
| HaCD00953227 | IgA | | Human adenovirus 41 | 27 | 87 |
| SgCD00811573 | IgG | pcsB | Streptococcus gallolyticus subsp. | 28 | 88 |
| | | | gallolyticus ATCC 43143 | | |
| HhCD00595176 | IgG | BVRF1 | Human herpesvirus 4 | 29 | 89 |
| SpCD00818032 | IgG | SP_2190 | Streptococcus pneumoniae | 30 | 90 |
| | | | TIGR4 | | |
| HpCD00780240 | IgA | HP0406 | Helicobacter pylori 26695 | 31 | 91 |
| HpCD00780836 | IgA | HP0373 | Helicobacter pylori 26695 | 32 | 92 |
| HmCD00952743 | IgG | L3 | Human mastadenovirus A | 33 | 93 |
| HmCD00849349 | IgG | L1 13.6k | Human mastadenovirus C strain | 34 | 94 |
| | | | human/USA/CL_42/1988/5[P5H5F5] | | |
| MtCD00544325 | IgG | PE_PGRS24 | Mycobacterium tuberculosis | 35 | 95 |
| | | | H37Rv | | |
| SpCD00875948 | IgA | SPy_1054 | Streptococcus pyogenes M1 GAS | 36 | 96 |
| HpCD00781213 | IgA | HP0923 | Helicobacter pylori 26695 | 37 | 97 |
| HpCD00849291 | IgA | N | Human parainfluenza virus 1 | 38 | 98 |
| HhCD00595168 | IgG | BDLF2 | Human herpesvirus 4 | 39 | 99 |
| SpCD00810863 | IgG | SP_0987 | Streptococcus pneumoniae | 40 | 100 |
| | | | TIGR4 | | |
| HsCD00959791 | IgG | HA | H3N2 subtype | 41 | 101 |
| HmCD00849346 | IgG | H648_37075gpHYPp1 | Human mastadenovirus C strain | 42 | 102 |
| | | | human/USA/CL_42/1988/5[P5H5F5] | | |
| PaCD00632105 | IgG | PA0572 | Pseudomonas aeruginosa | 43 | 103 |
| HmCD00849349 | IgG | L1 13.6 k | Human mastadenovirus C strain | 44 | 104 |
| | | | human/USA/CL_42/1988/5[P5H5F5] | | |
| SpCD00818023 | IgA | SP_2190 | Streptococcus pneumoniae | 45 | 105 |
| | | | TIGR4 | | |
| IcCD00953076 | IgG | M1 | Influenza C virus (C/Ann | 46 | 106 |
| | | | Arbor/1/50) | | |
| HmCD00956407 | IgG | L1 | Human mastadenovirus D | 47 | 107 |
| HpCD00780836 | IgA | HP0373 | Helicobacter pylori 26695 | 48 | 108 |
| MtCD00412192 | IgG | Rv1754c | Mycobacterium tuberculosis | 49 | 109 |
| | | | H37Rv | | |
| HmCD00849278 | IgG | G | Human metapneumovirus | 50 | 110 |
| SpCD00876414 | IgG | speB | Streptococcus pyogenes M1 GAS | 51 | 111 |
| HpCD00781182 | IgG | HP0528 | Helicobacter pylori 26695 | 52 | 112 |
| HrCD00959475 | IgA | | Human rhinovirus A1 | 53 | 113 |
| HpCD00781725 | IgG | HP1488 | Helicobacter pylori 26695 | 54 | 114 |
| HpCD00953108 | IgG | V | Human parainfluenza virus 4a | 55 | 115 |
| HpCD00780493 | IgG | oorA | Helicobacter pylori 26695 | 56 | 116 |
| HhCD00595131 | IgG | BFRF1 | Human herpesvirus 4 | 57 | 117 |
| HmCD00952869 | IgA | E3 | Human mastadenovirus D | 58 | 118 |
| SpCD00876354 | IgG | speH | Streptococcus pyogenes M1 GAS | 59 | 119 |
| EdCD00959595 | IgA | | Enterovirus D68 | 60 | 120 |

### II. Comparison Between Lung Adenocarcinoma Patients and Benign Nodule Controls

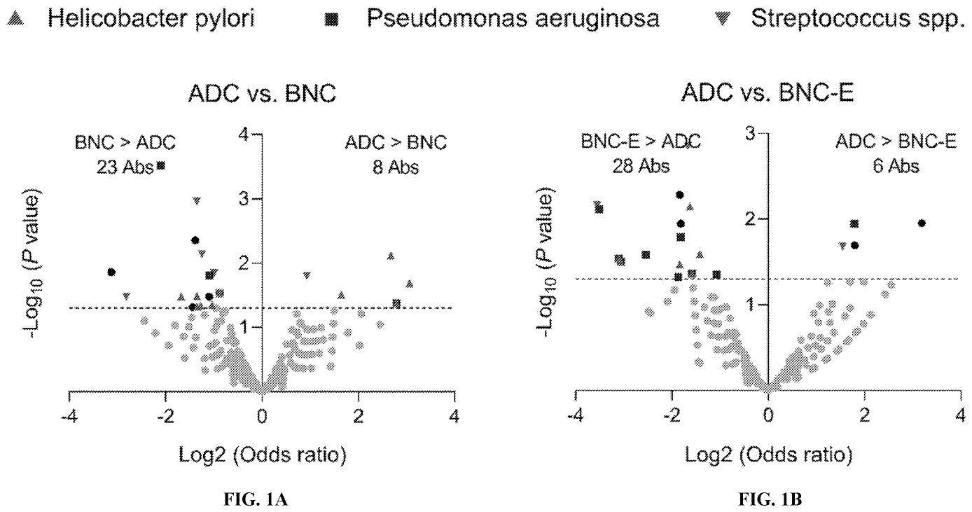

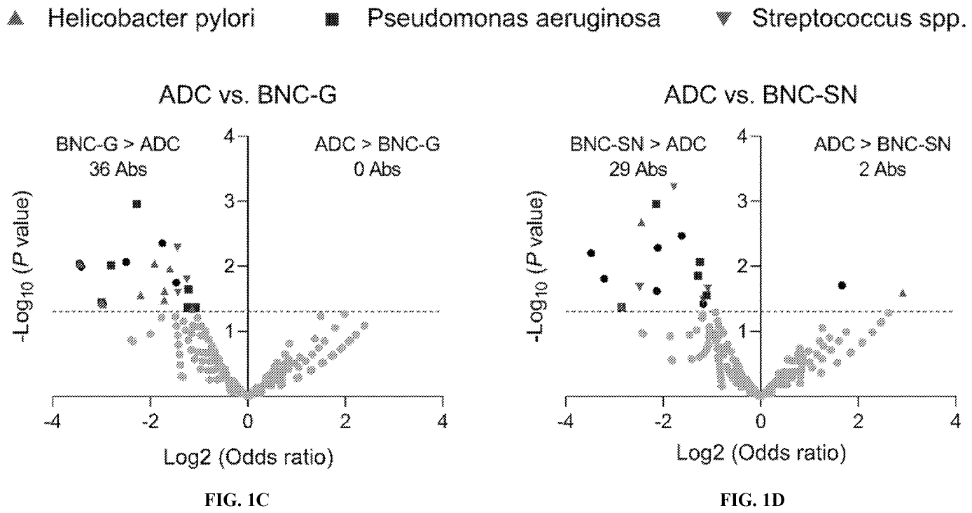

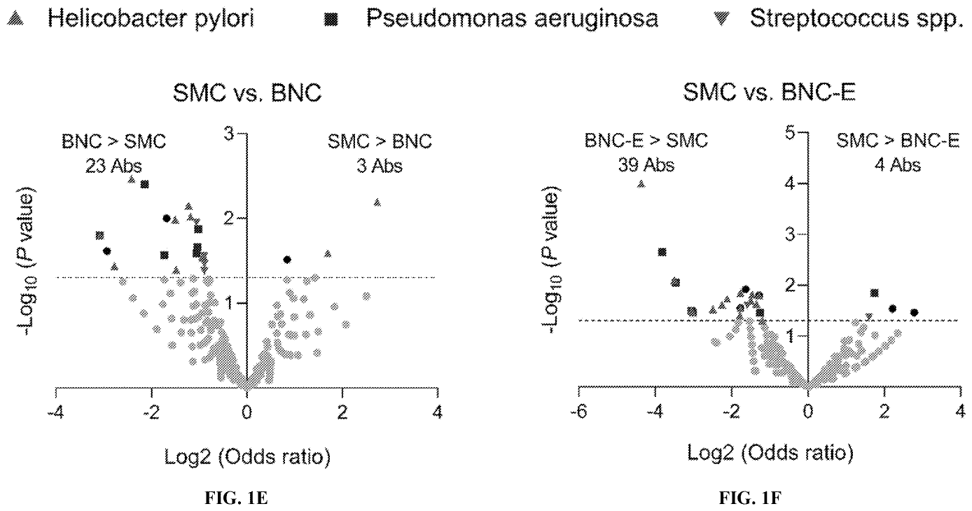

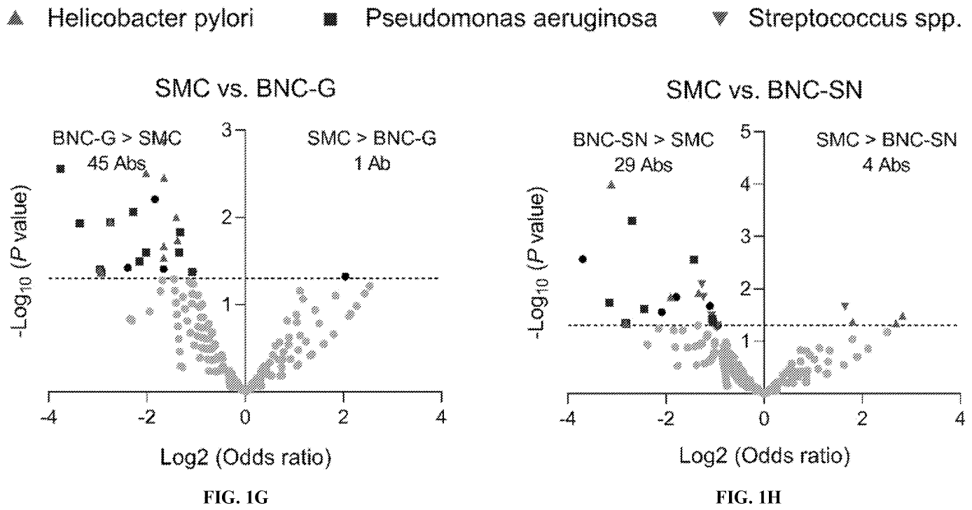

Anti-bacterial antibodies showing differential reactivity between ADC and BNC were further analyzed and identified more of such antibodies with higher prevalence among BNC than among ADC (Table 2). The p-values for the 4th quartile odds ratios (OR) for 31 anti-bacterial antibodies were less than 0.05; 23 had higher seroprevalence in BNC and only 8 were higher in ADC (two sample proportion test p-value <0.001) (FIG. 1A). When ADC was compared with each of the 3 subgroups of BNC individually, i.e., emphysema (BNC-E), granuloma (BNC-G), and stable nodule (BNC-SN), more antibodies had higher seroprevalences in each of the 3 BNC subgroups (FIG. 1B-D). The BNC-G subgroup and ADC had the greatest difference, 36 with higher prevalence in BNC-G vs. 0 in ADC (two sample proportion test p-value <0.001). The 3 subgroups of BNC showed heterogeneity in antibody profiles. However, overall differences of number of antibodies with higher seroprevalence in one BNC subgroup vs. the other was not observed (Table 5).

TABLE 5
Prevalence of anti-bacterial antibodies with odds ratio p-values <
0.05 in two comparison groups and their significance of differences.

| | | | | Two | |
|---|---|---|---|---|---|
| | | Number of | Number of | sample | |
| | | antibodies with | antibodies with | proportion | Number of |
| Groups | | higher prevalence | higher prevalence | test P | different |
| A | B | in A | in B | value | species |
| ADC | SMC | 21 | 18 | 0.650 | 12 |
| BNC-E | BNC-G | 2 | 7 | 0.059 | 6 |
| BNC-E | BNC-SN | 16 | 28 | 0.019 | 11 |
| BNC-G | BNC-SN | 9 | 8 | 1 | 5 |
| ADC with | ADC with | 7 | 10 | 0.492 | 9 |
| nodule | nodule size > | | | | |
| size <= 3 cm | 3 cm | | | | |
| Age <=65 | Age >65 | 29 | 40 | 0.088 | 10 |
| years | years | | | | |

ADC: Lung adenocarcinoma; BNC: Benign nodule controls; SMC: Smoker controls; BNC-E: Emphysema; BNC-G: Granuloma; BNC-SN: Stable nodule.

TABLE 6
Prevalence of anti-viral antibodies with odds ratio p-values <
0.05 in two comparison groups and their significance of differences.

| | | Number of | Number of | | Number |
|---|---|---|---|---|---|
| | | antibodies with | antibodies with | Two sample | of |
| Groups | | higher | higher | proportion | different |
| A | B | prevalence in A | prevalence in B | test P value | species |
| ADC | BNC | 3 | 12 | 0.003 | 9 |
| ADC | BNC-E | 5 | 11 | 0.077 | 12 |
| ADC | BNC-G | 14 | 13 | 1 | 19 |
| ADC | BNC-SN | 3 | 28 | <0.001 | 17 |
| ADC | SMC | 12 | 22 | 0.029 | 16 |
| BNC-E | BNC-G | 14 | 1 | <0.001 | 14 |
| BNC-E | BNC-SN | 5 | 21 | <0.001 | 16 |
| BNC-G | BNC-SN | 0 | 21 | <0.001 | 12 |
| SMC | BNC | 8 | 10 | 0.738 | 11 |
| SMC | BNC-E | 4 | 19 | <0.001 | 16 |
| SMC | BNC-G | 14 | 12 | 0.781 | 17 |
| SMC | BNC-SN | 5 | 32 | <0.001 | 16 |
| Light | Heavy | 11 | 13 | 0.772 | 13 |
| smokers | smokers | | | | |
| with <=20 pack- | with >20 | | | | |
| years | pack-years | | | | |
| smoking | smoking | | | | |
| history | history | | | | |
| BNC with | BNC with | 10 | 9 | 1 | 13 |
| nodule | nodule size > | | | | |
| size <= 1 cm | 1 cm | | | | |
| ADC with | ADC with | 13 | 5 | 0.019 | 13 |
| nodule | nodule size > | | | | |
| size <= 3 cm | 3 cm | | | | |
| Stage I ADC | Stage II and | 18 | 9 | 0.029 | 14 |
| | III ADC | | | | |

ADC: Lung adenocarcinoma; BNC: Benign nodule controls; SMC: Smoker controls; BNC-E: Emphysema; BNC-G: Granuloma; BNC-SN: Stable nodule.

### III. Comparison Between Smoker Controls and Benign Nodule Controls

BNC had more anti-bacterial antibodies than SMC (Table 2). When SMC was compared with BNC as a group, 26 anti-bacterial antibodies had OR p-values less than 0.05 with 23 higher in BNC and 3 higher in SMC (two sample proportion test p-value <0.001) (FIG. 1E). When SMC was compared with each of the 3 subgroups of the BNC individually, more antibodies had higher reactivity in all 3 subgroups (FIGS. 1F-1H). The BNC-G subgroup also had the greatest difference with SMC (45 vs. 1, p-value <0.001).

### IV. Comparison Between Lung Adenocarcinoma Patients and Smoker Controls

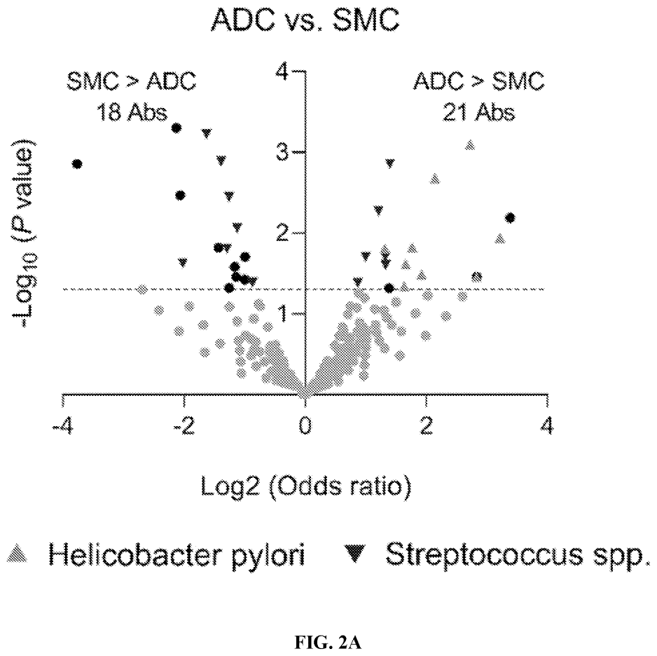

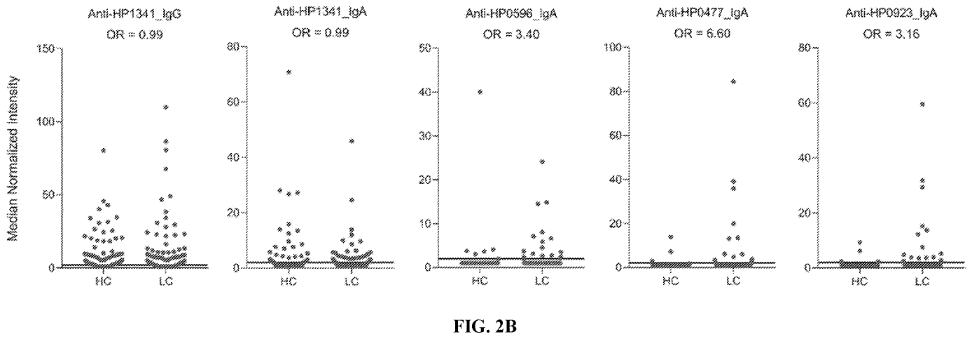

ADC and SMC had similar numbers of anti-bacterial antibodies. When ADC was compared with SMC, 39 anti-bacterial antibodies had the 4th quartile OR p-value smaller than 0.05 (FIG. 2A). Out of these 39 antibodies, the number of antibodies with higher seroprevalences in ADC was similar to that higher in SMC (21 vs. 18, two sample proportion test p-value=0.650) (Table 5). Among them, anti-H. pylori antibodies were significantly enriched in antibodies with significantly higher prevalence in ADC compared with SMC (11 higher vs. 0 lower, two sample proportion test p-value <0.001) (FIG. 2A). This was not observed for anti-Streptococcus spp. antibodies, which showed similar numbers of antibodies with significantly higher or lower prevalence in ADC relative to SMC (8 higher vs. 6 lower, two sample proportion test p-value=1).

Antibodies against 233 H. pylori were studied proteins. IgG and IgA antibodies to H. pylori protein HTP1341 had the highest reactivity among both ADC and SMC; however, neither showed a differential prevalence between ADC and SMC (FIG. 2B). Anti-H. pylori antibodies showing differences, such as anti-HP0596, anti-HP0923 and anti-HP0477, had lower overall reactivity compared with that for anti-HP1341 (FIG. 2B). Anti-H. pylori antibodies were also enriched in antibodies showing higher prevalence in BNC-G compared with ADC (FIG. 1C) or SMC (FIG. 1G). Similar to what was observed for antibodies showing significant differences in prevalence between ADC and SMC, anti-HP1341 had the highest overall IgG and IgA reactivity but did not show differences between BNC-G and ADC or SMC.

V. Association of Anti-Bacterial Antibodies with Clinical Parameters

Sub-group analysis was performed based on clinical parameters other than diagnosis to study the effects of smoking on the anti-bacterial antibody profiles (Table 2 and 5). Because most of the 420 study subjects were smokers, light smokers, subjects with <20 pack-years smoking history, and heavy smokers, subjects with >20 pack-years smoking history were compared. More anti-bacterial antibodies with higher seroprevalence in light smokers relative to heavy smokers were observed (51 vs. 2, two sample proportion test p-value <0.001, FIG. 8A). For BNC, Lower reactivity for subjects with ≤1 cm nodules than those with >1 cm nodules were observed (0 vs. 40, two sample proportion test p-value <0.001, FIG. 8B). However, differences comparing ADC with small nodules were not observed (≤3 cm) and ADC with large nodules (>3 cm) (7 vs. 10, two sample proportion test p-value=0.492) (Table 5). Different cutoffs of the nodule size were used when analyzing the benign and the case groups because the case group had overall larger nodules than the benign group. Slightly higher anti-bacterial reactivity was observed for ADC of stage I than for stages II or III (26 vs. 9, two sample proportion test p-value <0.001, FIG. 8C).

### VI. Correlation of Anti-Microbial Antibodies and Autoantibodies

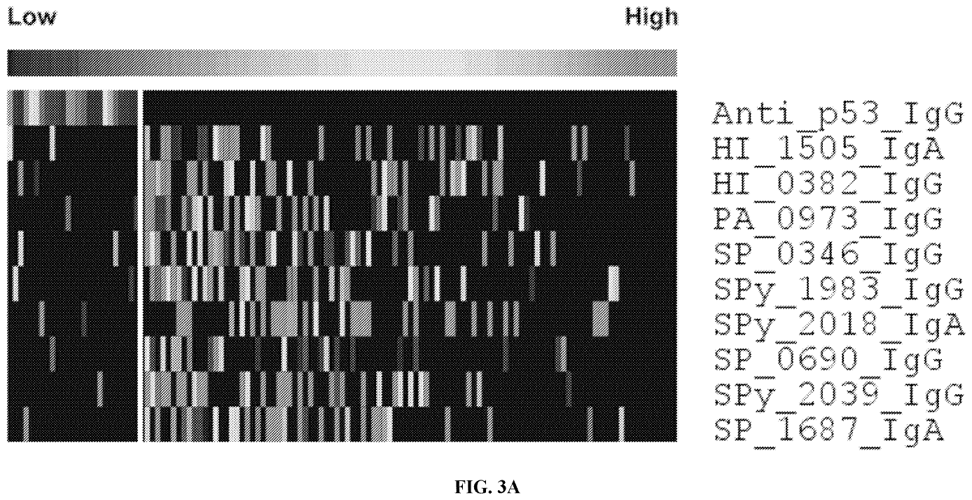

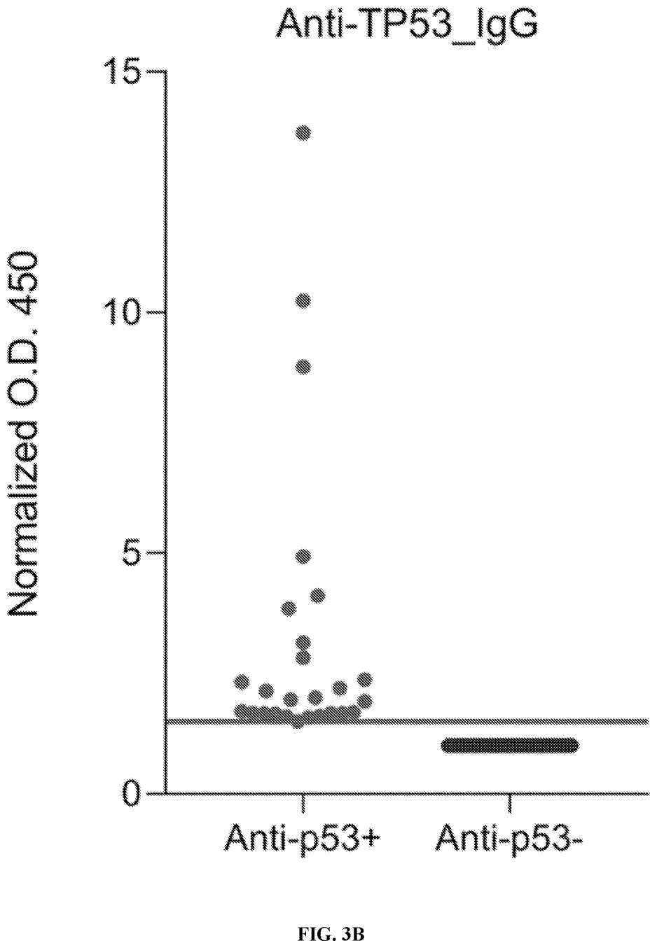

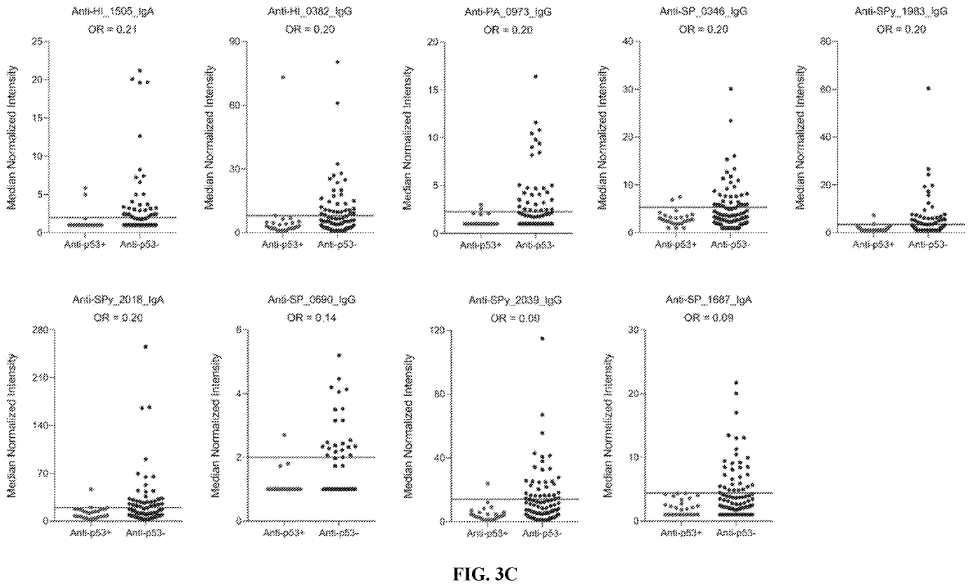

Target antigens for many cancer-specific antibodies play an important role in cancer development. Autoantibodies in the same set of samples were previously assessed by ELISA. Significant correlations with any anti-bacterial or anti-viral antibodies were not observed. p53 plays an important role in the development of many cancers, including lung cancer. Cancers with anti-p53 antibodies may have different pathogenesis from cancers related to microbial pathogens. Patients with anti-p53 antibodies usually carry p53 mutations, though the p53 mutation status for the study population was not known. Using anti-p53 seropositivity as a surrogate, anti-bacterial antibodies between anti-p53 positive and anti-p53 negative ADC were compared. Among the 9 antibodies showing significantly higher prevalence in anti-p53 negative ADC, 6 were against Streptococcus spp. and 2 were against Haemophilus influenzae (FIG. 3).

### VII. Panel Analysis Using Maximum Relevance-Minimum Redundance (MRMR) Logistic Regression

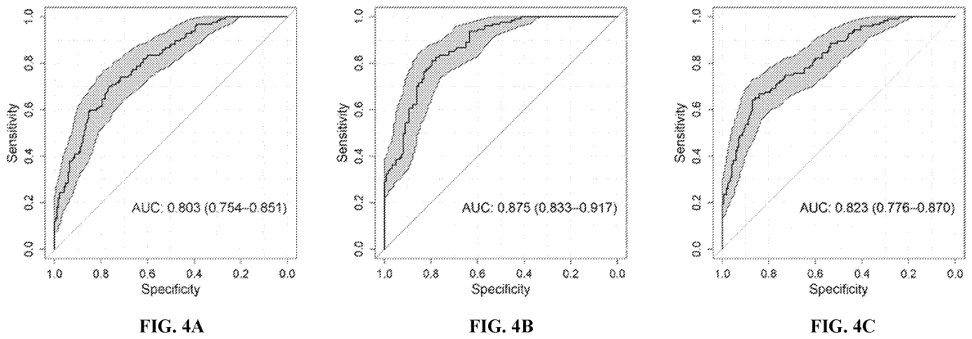

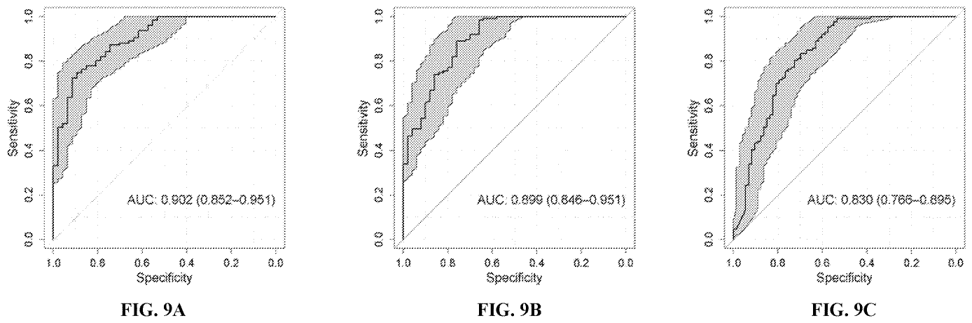

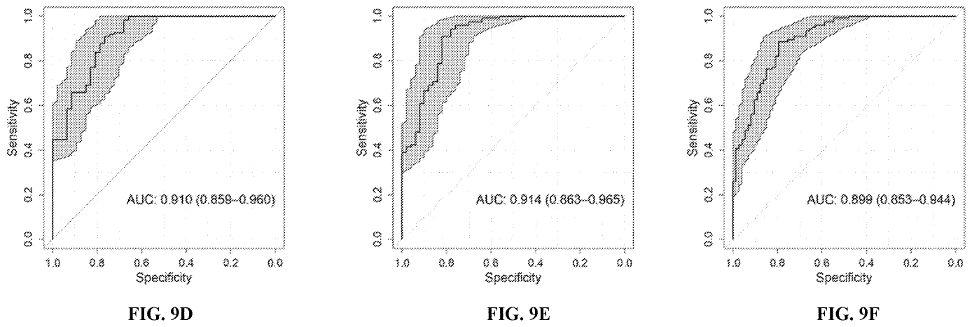

Antibody panels to distinguish ADC, SMC and BNC were conducted using anti-microbial antibodies with significantly different prevalence among these groups. MRMR logistic regression was used to build 20-antibody panels with area under the receiver operating characteristics curve (AUC) of 0.80 (95% confidence interval: 0.75-0.85), 0.88 (95% confidence interval: 0.85-0.92), and 0.84 (95% confidence interval: 0.79-0.88) distinguishing ADC vs. BNC, ADC vs. SMC, and BNC vs. SMC respectively (FIG. 4). The set of twenty antibodies used for each comparison is listed in Table 3. Table 4 lists the nucleic acid sequence encoding the antigen target of the sixty antibodies shown in Table 3 and the amino acid sequences of these antigen targets are shown in Table 4. When ADC and SMC were compared with the 3 BNC subgroups separately, anti-microbial antibodies could distinguish both ADC and SMC from BNC-E (FIGS. 9A and 9D) and BNC-G (FIGS. 9B and 9E) better than BNC-BN (FIGS. 9C and 9F).

The antibodies selected by MRMR algorithm is independent of the various comparisons performed in Table 2. Overall, anti-microbial antibodies could distinguish SMC from all 3 BNC subgroups better than ADC (FIGS. 9A-9F).

### VIII. Materials and Methods

a. Patients and Samples

All plasma samples were acquired from New York University with 127 from lung adenocarcinoma patients, 123 from gender matched smoker controls and 170 from benign nodule controls, including 47 emphysema, 50 granuloma and 70 stable nodule samples (Table 1). The ADC patients were recruited from the clinics at the NYU Cancer Center, and all gave informed consent for the institutional review board. The recruitment of high-risk smokers and benign nodule controls were also institutional review board approved.

b. Microbial Antigen Array Production and Anti-Microbial Antibody Profiling

Microbial antigen arrays were produced displaying 579 proteins from 27 bacteria and 322 proteins from 29 viruses, including both commensal and pathogenic microorganisms with an emphasis on those associated with respiratory tract infection (Tables 7 and 8) using the Nucleic Acid Programmable Protein Array (NAPPA) technology to assay anti-microbial antibodies in plasma samples. Microbial genes in pANT7_cGST, an expression vector that allows gene transcription from the T7 protomer and protein expression using HeLa lysates based in vitro expression system, were ordered from the plasmid repository DNASU (DNASU.org). Microbial antigen NAPPA was produced by spotting plasmid DNA on silicon nanowell substrates as described. Antibody profiling was also performed as previously reported. In brief, on the day of antibody profiling, c-terminal GST tagged microbial antigens were expressed using HeLa cell lysates-based in vitro expression system (ThermoFisher Cat #88881) and in situ captured by co-spotted anti-GST antibody (GE Healthcare Cat #27-4577-01) for their display on microbial antigen arrays. Plasma samples were randomized and applied on the microbial antigen arrays to reduce potential bias. Antibodies in plasma samples against displayed antigens were detected by ani-Human IgG (Jackson ImmunoResearch Labs Cat #109-605-008) and anti-Human IgA (Jackson ImmunoResearch Labs Cat #109-165-011) antibodies labelled with different fluorophores. Arrays were scanned, and the images were analyzed by the Array-Pro image analysis software (Media Cybernetics). Median fluorescence intensities at each spot were calculated for downstream data analysis.

TABLE 7
Bacteria studied on microbial protein arrays.

| Strain | Phylum | Number of proteins |
|---|---|---|
| Helicobacter pylori | Proteobacteria | 233 |
| Pseudomonas aeruginosa | Proteobacteria | 97 |
| Streptococcus pneumoniae | Firmicutes | 57 |
| Haemophilus influenzae | Proteobacteria | 26 |
| Streptococcus pyogenes | Firmicutes | 25 |
| Mycobacterium tuberculosis | Actinobacteria | 18 |
| Streptococcus gallolyticus | Firmicutes | 18 |
| Fusobacterium nucleatum | Fusobacteria | 17 |
| Klebsiella pneumoniae | Proteobacteria | 17 |
| Escherichia coli | Proteobacteria | 7 |
| Gemella haemolysans | Firmicutes | 7 |
| Bacteroides vulgatus | Bacteroidetes | 7 |
| Campylobacter jejuni | Proteobacteria | 7 |
| Shigella flexneri | Proteobacteria | 6 |
| Bacteroides fragilis | Bacteroidetes | 6 |
| Peptostreptococcus anaerobius | Firmicutes | 5 |
| Citrobacter koseri | Proteobacteria | 5 |
| Leptotrichia buccalis | Fusobacteria | 4 |
| Clostridium difficile | Firmicutes | 4 |
| Enterococcus faecalis | Firmicutes | 3 |
| Porphyromonas gingivalis | Bacteroidetes | 3 |
| Eubacterium rectale | Firmicutes | 2 |
| Dorea formicigenerans | Firmicutes | 1 |
| Bifidobacterium adolescentis | Actinobacteria | 1 |
| Prevotella copri | Bacteroidetes | 1 |
| Faecalibacterium prausnitzii | Firmicutes | 1 |
| Veillonella parvula | Firmicutes | 1 |

TABLE 8
Viruses studied on microbial protein arrays.

| Strain | Phylum | Number of proteins |
|---|---|---|
| Human herpesvirus 4 | Peploviricota | 46 |
| Human mastadenovirus C | Preplasmiviricota | 40 |
| Influenza A virus | Negarnaviricota | 33 |
| Human herpesvirus 5 | Peploviricota | 29 |
| Human mastadenovirus F | Preplasmiviricota | 23 |
| Human coronavirus OC43 | Pisuviricota | 12 |
| Enterovirus C | Pisuviricota | 12 |
| Human mastadenovirus B | Preplasmiviricota | 12 |
| Rhinovirus B | Pisuviricota | 10 |
| Human parainfluenza virus 1 | Negarnaviricota | 10 |
| Human metapneumovirus | Negarnaviricota | 10 |
| Human respiratory syncytial virus B | Negarnaviricota | 9 |
| Human coronavirus 229E | Pisuviricota | 8 |
| Enterovirus D | Pisuviricota | 8 |
| Human mastadenovirus A | Preplasmiviricota | 8 |
| Rhinovirus C | Pisuviricota | 7 |
| Human mastadenovirus D | Preplasmiviricota | 7 |
| Human rhinovirus A | Pisuviricota | 7 |
| Human orthopneumovirus | Negarnaviricota | 6 |
| Human bocavirus 1 | Cossaviricota | 4 |
| Human coronavirus HKU1 | Pisuviricota | 4 |
| Human coronavirus NL63 | Pisuviricota | 4 |
| Human parainfluenza virus 4 | Negarnaviricota | 4 |
| Small anellovirus 1 | Unclassified phylum | 3 |
| Influenza C virus | Negarnaviricota | 2 |
| Influenza B virus | Negarnaviricota | 1 |
| Human bocavirus 3 | Cossaviricota | 1 |
| Human bocavirus 2 | Cossaviricota | 1 |
| Human bocavirus 4 | Cossaviricota | 1 |

c. Statistical Analysis

Spot intensities on each array were normalized by dividing by the median spot intensity of the corresponding array before statistical analysis to minimize the effects of the overall background differences among samples. Seropositivity was determined using the empirical median normalized intensity cutoff 2 as previously reported. Descriptive statistics for demographic and clinical variables were calculated. The 4th quantile odds ratio (OR) of 2 comparison groups was computed for each antibody based on the maximum of the empirical technical cutoff value 2 and 75th percentile of all samples, and p-values were computed using the chi-square test. Volcano plots of negative logarithm base 10 of p-values versus logarithm base 2 of OR were generated for each comparison. Autoantibody seropositivity were determined using an empirical cutoff 1.5 for the ELISA data previously reported. The proportions of antibodies having P values less than 0.05 and OR less than and greater than 1 were compared using two-sample proportion test.

Antibody panels were built using logistic regression of antibodies selected using the Maximum Relevance-Minimum Redundance (MRMR) feature selection algorithm, and the classification performance was evaluated using the ROC generated with pROC package available for R (v4.1.0). d. Bioinformatics analysis

Correlation between antibody reactivity was measured by the Spearman rank correlation coefficient. Correlation heatmaps were generated with Pandas (v1.3.4) and Seaborn (v9.11.2) packages available in Python (v3.8.10).

1. An antibody panel for diagnosing whether a subject has lung carcinoma, the antibody panel comprising at least one antigen from at least one bacterium selected from the group consisting of: Helicobacter pylori, Pseudomonas aeruginosa, Streptococcus pneumoniae, Haemophilus influenzae, Streptococcus pyogenes, Mycobacterium tuberculosis, Streptococcus gallolyticus, Fusobacterium nucleatum, Klebsiella pneumoniae, Escherichia coli, Gemella haemolysans, Bacteroides vulgatus, Campylobacter jejuni, Bacteroides fragilis, Peptostreptococcus anaerobius, Citrobacter koseri, Leptotrichia buccalis, Clostridium difficile, Enterococcus faecalis, Porphyromonas gingivalis, Bifidobacterium adolescentis, Prevotella copri, Faecalibacterium prausnitzii, and Veillonella parvula.

2. The antibody panel of claim 1, wherein the antibody panel further comprises at least one antigen from at least one virus selected from the group consisting of: human herpesvirus, human mastadenovirus, influenza A virus, human coronavirus OC43, enterovirus, rhinovirus B, human parainfluenza virus 1, human metapneumovirus, human respiratory syncytial virus B, and human coronavirus 229E.

3. The antibody panel of claim 2, wherein the virus is selected from the group consisting of: human herpesvirus 4, human mastadenovirus C, influenza A virus, human herpesvirus 5, human mastadenovirus F, human coronavirus OC43, enterovirus C, human mastadenovirus B, rhinovirus B, human parainfluenza virus 1, human metapneumovirus, human respiratory syncytial virus B, human coronavirus 229E, and enterovirus D.

4. The antibody panel of claim 1, wherein the antibody panel comprises at least one antigen from at least one bacterium selected from the group consisting of: Campylobacter jejuni, Enterococcus faecalis, Helicobacter pylori, Pseudomonas aeruginosa, Streptococcus pneumoniae, Haemophilus influenzae, Streptococcus pyogenes, Mycobacterium tuberculosis, Streptococcus gallolyticus, and Fusobacterium nucleatum.

5. The antibody panel of claim 2, wherein the antibody panel comprises at least one antigen from at least one virus selected from the group consisting of: human herpesvirus 4, human mastadenovirus C, influenza A virus, human herpesvirus 5, human mastadenovirus F, and Human coronavirus OC43.

6. (canceled)

7. (canceled)

8. The antibody panel of claim 1, wherein the antibody panel comprises at least 20 antigens having the amino acid sequences set forth in SEQ ID NOs. 61-120.

9. The antibody panel of claim 1, wherein the antibody panel comprises at least 20 antigens and the antigens are encoded by the nucleotide sequences set forth in SEQ ID NOs. 1-20.

10. The antibody panel of claim 1, wherein the antibody panel comprises at least 20 antigens and the antigens have the amino acid sequences set forth in SEQ ID NOs. 61-80.

11. The antibody panel of claim 1, wherein the antibody panel comprises at least 20 antigens and the antigens are encoded by the nucleotide sequences set forth in SEQ ID NOs. 21-40.

12. The antibody panel of claim 1, wherein the antibody panel comprises at least 20 antigens having the amino acid sequences set forth in SEQ ID NOs. 81-100.

13. The antibody panel of claim 1, wherein the antibody panel comprises at least 20 antigens encoded by the nucleotide sequences set forth in SEQ ID NOs. 41-60.

14. The antibody panel of claim 1, wherein the antibody panel comprises at least 20 antigens having the amino acid sequences set forth in SEQ ID NOs. 101-120.

15. A method of diagnosing lung carcinoma in a subject, the method comprising:
providing a serum or blood sample from the subject;
contacting the serum or blood sample with the antibody panel of claim 1;
detecting the presence of any antibody against the at least one antigen or antigens in the antibody panel; and
determining the subject is likely to have lung carcinoma upon the detection of at least one antibody that binds to an antigen having an amino acid sequence selected from the group consisting of: SEQ ID NOs. 65, 66, 69, 70, 72, 75, 76, 81, 86, 87, 91, 92, and 95-98.

16. The method of claim 15, wherein the serum or blood sample from the subject is contacted with the antigen panel of claim 9, the subject is determined to have lung carcinoma upon the detection of antibodies that bind to antigens having amino acid sequences set forth in SEQ ID NOs. 65, 66, 69, 70, 72, 75, and 76.

17. The method of claim 15, wherein the serum or blood sample from the subject is contacted with the antigen panel of claim 12, the subject is determined to have lung carcinoma upon the detection of antibodies that bind to antigens having amino acid sequences set forth in SEQ ID NOs. 81, 86, 87, 91, 92, and 95-98.

18. A method of diagnosing lung carcinoma in a subject, wherein Computer Tomography (CT) scanning of the subject identified an indeterminate pulmonary nodule, the method comprising:
providing a serum or blood sample from the subject; and
contacting the serum or blood sample with the antibody panel of claim 1,
wherein detecting the presence of an antibody against at least one antigen in the antibody panel in the serum or blood sample selected from the group consisting of an antigen encoded by SEQ ID NOs. 5, 6, 9, 10, 12, 15, 16, 21, 26, 27, 31, 32, and 35-38 indicates the indeterminate pulmonary nodule in the subject is lung carcinoma.

19. The method of claim 18, wherein the serum or blood sample is contacted with the antibody panel of claim 8.

20. The method of claim 18, wherein detecting the presence of an antibody against at least one antigen in the antibody panel in the serum or blood sample selected from the group consisting of an antigen encoded by SEQ ID NOs. 5, 6, 9, 10, 12, 15, 16, 21, 26, 27, 31, 32, and 35-38 indicates the indeterminate pulmonary nodule in the subject is lung adenocarcinoma.

21. The method of claim 18, further comprising detecting the presence of an antibody against at least one antigen encoded by SEQ ID NOs. 1-4, 7, 8, 11, 13, 14, 17-20, 43, 45, 46, 48, 49, 51, 53-57, and 59-60 in the serum or blood sample indicates the subject has a benign nodule.

22. The method of claim 18, wherein a blood sample is provided from the subject.

23-32. (canceled)


SEQUENCE LISTING (120 sequences)

SEQ ID NO: 1 - DNA, 1053 bp, Streptococcus gallolyticus
  source 1..1053: mol_type = genomic DNA
atgaagaaaaaactagtaattgttggtgctctaagtgctctggttttacctttgttcttggttgttattatcatgggagcttttgcgggtagtgcgtcttctaatggacaagccaaaggcctacaaacgaaactaatcgctaaagaagtcgctcaaaaagctaatatttcagaagaacgggctagtgatgtcattaaaattctgaattatgaactttcacaagaacaatttactatcgcaggagcttctggaagtttggcagttgcggaacgtgaaagtggctttgaccctaaagccgtcaatacaggtggtggtgtggctggttatttccaatggtcgggctggtcttctactatcaatggcaatcgctggtcaaaggcaaaacaaaagaaattagattctgatattgaactagacctcttatctgccgagctaaacagctcttatagcaaagtcaaaacagaaatgcaaaaaacgactgaccctgaaaaggcagccctttattggtcagaacattatgaaggggtcgctttatcagacggtcaaacaaaagctagtgacctgaaaaagaatgcgagaaaatggtataatctcttcaaagatactttaacaggtgatacagccaatgccaaaacacgagacggtggaggcgtaacttctgatggagtgcctgccggatatagccttacaaaggcaatcaacacctctaactatacttctgcgacctatccttggggacaatgtacatggtttgtctataaccgagccaaagaggtaggcgttaactttgacccttatatggggaatggcggggattggaagcagaaagcaggctaccaaaccacacatacaccaactgaacatagtgctcttagcttctctcctggacaagctggcgcagacccaacttacgggcacattgcgtttgtggagcaggtcaaatcagacgggtctattctcatttcagaagccaacgccaaaggacttggagttgtttcttatcgcacatttgataaggtaacagctaaccaatttacatacgtaattgggaag

SEQ ID NO: 2 - DNA, 711 bp, Pseudomonas aeruginosa
  source 1..711: mol_type = genomic DNA
atgttcacctcccgttgcctgcctctggccgcagccgtcaccgcactggcgctgctcgccggttgtgccaacaacaacccctacgacacgcaaagccaaagccagggtggcatgagcaagacggccaagtacggcggactgggtgcgctggccggcgccgtcgccggcgctgcgatcgaccacaacaaccgtggcaagggcgcgctgatcggcgctgccgttgccggcgcggccgccgcgggttatggctactacgccgacaagcaggaagccgagctgcgtcggcagatggaaggcaccggcgtggaagtgcagcgccaaggtgacgacatcaagctgatcatgccgggcaacatcaccttcgccaccgattcggcgaacatcgccccgagcttctacgcgccgctgaacaacctggcgaactcgttcaagcagtacaaccagaacaccatcgagattgtcggttacaccgacagcaccggcagccgccagcacaacatggacctgtcccagcgtcgtgcgcagagcgtggccggctacctgaccgcccagggcgtcgacggcacccgcctgagcacccgcggcatgggcccggaccagccgatcgcgagcaactccactgccgacggtcgcgcgcagaaccggcgcgtcgaggtcaacctgcgaccggttccgggcgcccagggcccggcgcagacccagccgcagtac

SEQ ID NO: 3 - DNA, 834 bp, Helicobacter pylori
  source 1..834: mol_type = genomic DNA
atggagcgttcgcttatttttaaaaaagttagaatttattctaaaatgttagtggctttagggctttcaagcgtgttgatcggttgcgcgatgaatccaagcgctgaaacaaaaacaccaaatgacgccaaaaatcaagttcaaactcatgaaagaatgaaaacaagctctgaacatgttacgccgctagattttaattatccgatacatattgttcaagccccacaaaaccaccatgttgtaggtattttaacaccgcgcattcaagtgagcgataatctaaaaccctatattgataagtttcaagacgctttaattaatcaaatccaaactatttttgaaaaaagaggctatcaagtgttgcgttttcaagatgaaaaagctttaaatgcgcaagataagagaaagattttttccgttttggatttgaaagggtgggtaggaatcttagaagatttgaaaatgaatttaaaagatcccaataatcctaatttagacacgctagtggatcaaagctcaggctctgtgtggtttaatttttatgagccagaaagcaatcgtgtagtccatgattttgctgtggaagtagggacttttcaggcaatgacctatacttacaaacacaataattctggagggcttaattcttcaaacagcattatccatgaatatttggaaaagaataaagaagatgcgatacataagatcttaaacagaatgtatgcggttgtcatgaaaaaagctgtaacagaacttacaaaagaaaatattgacaaatacagagaggctattgatagaatgaaaggctttaaaagttctatgcctcaaaaaaag

SEQ ID NO: 4 - DNA, 1713 bp, Human herpesvirus 4
  source 1..1713: mol_type = genomic DNA
atggcgctctccgggcacgtcttgatagaccccgcccgactgcccagggacacaggccccgaactcatgtgggccccaagccttcgcaactcactgcgcgtgtcccccgaagcgctcgagctggcagagcgggaggccgaaagggccaggtcggagcggtgggacaggtgtgcccaggtgctcaaaaataggctgctccgcgtggagctggacggcatcatgcgtgaccacctggccagggcggaggagatccgccaggacctggatgctgtagtggccttctctgatggcctggagagcatgcaggtcaggtccccctccacgggagggcgctctgcgccagccccgccctccccatccccagcccagccgttcactcggctcaccgggaacgcccagtatgcagtctcaatctctcccacggacccccctctgatggtggccggcagcctggctcaaacgctgcttggtaatctgtacgggaacatcaaccagtgggtaccgtccttcggaccctggtacaggaccatgtcggctaatgccatgcagcggcgcgtgttccctaagcagctgaggggcaacctgaactttaccaactccgtctccctaaagctgatgacagaagtggtggcggtgcttgagggcaccacccaggactttttctcagacgtcaggcacctgccagacctccaggctgccctgatcctctcggtggcctacctgctactccaggggggctcctcacaccagcagcgccccctccctgcctcacgggaagagctgctggagctgggcccggagagcctagagaaaatcatcgccgacctcaaggccaagtcacccggcggaaattttatgattttaacaagcggaaacaaggaagcgcgccagtcaatagcccctctcaaccgacaggcggcatatccacccggcacattcgcggacaataagatttacaacctgtttgtgggagcgggactactgcccacgacggccgcgctgaacgtgcccggggcggcgggtcgggaccgggacctggtgtaccggatcgccaaccagatctttggggaggatgtgccccccttctcatctcaccagtggaacctgcgcgtaggtttagccgcactcgaggccctgatgctcgtctacacgctctgcgagaccgccaacctggccgaggcggccacccggcgtctacacctatcgtccctgctcccccaggcaatgcagcggcgcaagcctgccatggcgtcagctggtatgccgggcgcctatccagtccagacgcttttccgccacggggagctcttccgcttcatctgggcccactacgtgaggcccacggtggcggcagacccccaggcctccatcagctctcttttccccgggctggttttgctggccctggagctgaagttgatggatgggcaggctccctcccattatgccataaacctgaccggacaaaagtttgacaccctctttgagattatcaaccagaagcttttatttcacgacccggctgccatgctggcggcgcgcacacagctgcgtctagccttcgaggacggcgtcggtgttgccctggggcgcccctcgcccatgcttgcggcgcgggagatcctggagcgtcagttctcagcctcggatgactacgaccggctgtacttcctgacgctgggctacctggcctccccggtggccccaagctga

SEQ ID NO: 5 - DNA, 552 bp, Helicobacter pylori
  source 1..552: mol_type = genomic DNA
atgaaaaaaatggttttggtatcggttttactagcagggtttttgcaagcggtgaatttggatttatcttcggctaagctaacatggacagcctttaaaactaaggctaaaacaccagtaaatgggagttttgaaagcatcacctataaattgggtaaatctcaagatagtttaaaaacccttttagagggagcgagcgcgagcatggatagcttgaaagtcaatttaggcgatgaattgaaaaacaaaaatgtgaaagaagcttttttcgctctttttaaaaacactaacatcaaagtaactttcaggaatgtgatagaaggcgatcatgcaggttctcttacggcttatgtgagaatgaatgaaaagctggtgaaagtgcctatgcaatacacgattgctgaggataagatcgtggttaaaggggttttggatttattgaattttggcttgaaaaacgaattagcgagcttggccaaacgatgcgaaagctttcatgagggcttgacttggtcgcaagtggaaatccaatttgaaagcatgatcaaggga

SEQ ID NO: 6 - DNA, 1065 bp, Helicobacter pylori
  source 1..1065: mol_type = genomic DNA
atgtcaggaattagcgctcaattagtcaaaaaattaagggacttaaccgatgcgggcatgatggattgcaaaaaagcccttgtagaagtggctggggatttgcaaaaggctattgatttcttgcgcgaaaaaggcttgagtaaagccgctaaaaaagccgataggatcgctgctgagggcgttgtcgctttagaagtagcgcctgattttaaaagcgcaatgatagtagaaatcaatagcgaaacggattttgtggctaaaaatgagggctttaaggaattggtgaaaaaaactttagaaacgatcaaagcccacaatatccataccacagaagaactgcttaaaagcccgttagacaacaagccttttgaagaatatttgcactctcaaatcgctgtgattggtgaaaacattttagtgagaaaaatcgctcatttaaaagcccccagctctcatatcatcaatggttatgcgcattctaacgctagagtgggcgtgttaatcggtataaaatacgataatgaaaaaaacgctccaaaagtggtggaactggcccgaaacatcgctatgcatgccgcagcgatgaaacctcaagtgctagattgcaaagactttagccttgattttgtcaaaaaagaaactttagccttgatcgctgaaattgaaaaagacaatgaggaagctaaacgcttgggcaaacctttaaaaaacatccccacttttgggagccgcattgaattgagcgatgaagttttagcccatcaaaagaaagcctttgaagacgaattaaaagcacaaggcaagcctgaaaaaatctgggataaaatcgttcctggaaaaatggaaagatttatcgctgataacacccttattgatcaacgcctgactcttttagggcaattctatgtcatggacgataaaaaaactatcgctcaagtggttgctgattgttccaaagaatggaatgatgatttaaaaatcactgagtatgtgcgttttgaattgggcgaaggcattgagaaaaaggcagagaatttcgctgaagaagtggctttgcaaatgaag

SEQ ID NO: 7 - DNA, 606 bp, Gemella haemolysans
  source 1..606: mol_type = genomic DNA
atggtggttaagtatgtagatgaaaatggaaaagacttaattcctgcagaaacaacagaaggaaaagaaggagatgagtacactacaagtggaaaagtgattccaggttatgtgcttgttcgtgtagatggagaagctaaaggtaagattggaaaagatggttcaacagtaacatacgtttacaaaccattaggatcatgggtaacaaacatcccaggacaaccaacaagccaaattaaatatccaaacgacccaacagatccaacaaaaccaggaagtgaaagaccggtattaccatacgtaccaggatatactccagttgatggaaatggaaatccacttaaaccagttgatccacaagacccaactaaaggatatatcataccagatatccctacaaacccaggtcaagatacagtaatcaactatgtggcaaatccaaaaccacaaccaaaacaagatcaaaaaccagttcaaccaaaagcaaatgctcaagttaagagattagctaacacaggtgcaactgaaactaatactggattagcaggattaggacttgcggcacttggtggaatgcttgcagcggtaagacgtcgtaaagaaaaa

SEQ ID NO: 8 - DNA, 555 bp, Streptococcus pneumoniae
  source 1..555: mol_type = genomic DNA
atgaaaaaagtaatgtttgctggcttaagtctcttgtcattagttgtattgatggcctgtggtgaggaagaaactaaaaagactcaagcagcacaacagccaaaacaacaaacgactgtacaacaaattgctgttggaaaagatgctccagacttcacattgcaatccatggatggcaaagaagttaagttatctgattttaagggtaaaaaggtttacttgaagttttgggcttcatggtgtggtccatgcaagaaaagtatgccagagttgatggaactagcggcgaaaccagatcgtgatttcgaaattcttactgtcattgcaccaggaattcaaggtgaaaaaactgttgagcaattcccacaatggttccaggaacaaggatataaggatatcccagttctttatgataccaaagcaaccaccttccaagcttatcaaattcgaagcattcctacagaatatttaattgatagccaaggaaagattggaaagattcaatttggtgctatcagtaatgcggatgcagaagcagcatttaaagaaatgaac

SEQ ID NO: 9 - DNA, 1452 bp, Streptococcus pyogenes
  source 1..1452: mol_type = genomic DNA
atggccaagaacaatacaaacaggcactacagtctgcgcaagctgaagaccggcacagcctcagttgcagtggctctgaccgtgctgggtgcaggtttcgctaatcagactgaggtcaaggccaacggagatggtaatccccgggaagtgatcgaagacctggccgctaacaatcctgccatccagaacattcggctgagatacgagaacaaggacctgaaggctagactggagaacgctatggaagtggcagggcgagactttaagcgtgccgaggaactggagaaagcaaagcaggccctggaagaccagagaaaggatctggagactaaactgaaggagctgcagcaggactatgatctggccaaagagtctaccagttgggataggcagcggctggagaaggagctggaggaaaagaaagaggccctggagctggccatcgaccaggcatctcgggattaccatagagcaaccgccctggagaaagagctggaggagaagaagaaggccctggagctggccatagaccaggcaagtcaggattataacagggccaacgtgctggagaaagagctggaaactatcacccgagagcaggaaattaaccgtaatctgctcggcaatgccaagctggagctggaccagctgagcagcgagaaggaacagctgacaatcgagaaagcaaagctggaggaagagaagcagatttccgatgccagccggcagagcctgcgtcgggacctggacgccagccgagaggctaaaaagcaggtggaaaaagacctggcaaacctgactgccgagctggacaaagtcaaggaagataagcagatcagcgacgccagccggcagggcctgaggcgcgacctggatgccagccgggaggcaaaaaagcaggttgaaaaggacctggccaacctgaccgctgagctggacaaagtgaaagaggagaagcagatttccgacgcctctaggcagggcctgcgacgggacctggacgcatcccgcgaggccaaaaagcaggtcgaaaaagctctggaagaggcaaacagcaagctggccgccctggagaaactgaataaggaactggaagagtccaaaaagctgacagagaaagaaaaggccgagctgcaggctaaactggaggctgaagcaaaagccctgaaggagcagctggctaagcaggcagaagagctggccaaactgcgggccggaaaggcttcagatagccagacacccgacactaaacctggtaacaaggccgtgccaggcaagggtcaggcaccacaggcaggcaccaaacccaaccagaataaggcccctatgaaagagacaaagaggcagctgccatctaccggcgaaacagctaaccccttcttcactgctgcagccctgactgtcatggctaccgcaggggttgctgcagtggtcaaacgcaaggaagagaat

SEQ ID NO: 10 - DNA, 747 bp, Pseudomonas aeruginosa
  source 1..747: mol_type = genomic DNA
atgaggcgaacggtgaaaggtctgtcgcgcatggcgctcctggcgctggtcctggcgttgggcgggtgcaaggtcgaactctataccgggatcagccagaaggaaggcaacgagatgctggcgctgttgcgttcggagggcgtttccgcggacaagcaggcggacaaggatggcaccgtgcgcctgctggtggaggaatcggatatcgccgaggcggtggaggtgctcaagcgcaagggctatccgcgggagaacttctccacgctgaaggatgtgtttcccaaggacgggctgatttcctcgcccatcgaagagcgcgcgcggctcaactacgccaaggcccaggagatttcccataccctttccgagatcgatggcgtgctggtggcgcgggtccatgtggtgctgcccgaggagcgcgacggactgggcaggaagtcctcgccggcatcggcctcggtgttcatcaagcacgccgccgacgtgcaactggatgcctacgtgccgcagatcaagcaactggtgaacaacggcatcgaaggactcagctacgaccgcatcagcgtggtcctggtgccgtcggccggggtccgccaggtcccgctggcgccacgcttcgagagtgtcttctcgatccaggtggcggagcactcgcgcggacgcctgctcggcctgttcggcctgctgctggcgttgctgctggcgagcaacctggcgcagttcttctggcaccggcaacggggctga

SEQ ID NO: 11 - DNA, 1413 bp, Streptococcus pneumoniae
  source 1..1413: mol_type = genomic DNA
atggcaaataaagcagtaaatgactttatactagctatgaattacgataaaaagaaactcttgacccatcagggagaaagtattgaaaatcgtttcatcaaagagggtaatcagctacccgatgagtttgttgttatcgaaagaaagaagcggagcttgtcgacaaatacaagtgatatttctgtaacagctaccaacgacagtcgcctctatcctggagcacttctcgtagtggatgagaccttgttagagaataatcccactcttcttgcggtcgatcgtgctccgatgacttatagtattgatttgcctggtttggcaagtagcgatagctttctccaagtggaagaccccagcaattcaagtgttcgcggagcggtaaacgatttgttggctaagtggcatcaagattatggtcaggtcaataatgtcccagctagaatgcagtatgaaaaaatcacggctcacagcatggaacaactcaaggtcaagtttggttctgactttgaaaagacagggaattctcttgatattgattttaactctgtccattcaggcgaaaagcagattcagattgttaattttaagcagatttattatacagtcagcgtagatgctgttaaaaatccaggagatgtgtttcaagatactgtaacggtagaggatttaaaacagagaggaatttctgcagagcgtcctttggtctatatttcgagtgttgcttatgggcgccaagtctatctcaagttggaaaccacgagtaagagtgatgaagtagaggctgcttttgaagctttgataaaaggagtcaaggtagctcctcagacagagtggaaacagattttggacaatacagaagtgaaggcggttattttagggggcgacccaagttcgggtgcccgagttgtaacaggcaaggtggatatggtagaggacttgattcaagaaggcagtcgctttacagcagatcatccaggcttgccgatttcctatacaacttcttttttacgtgacaatgtagttgcgacctttcaaaatagtacagactatgttgagactaaggttacagcttacagaaacggagatttactgctggatcatagtggtgcctatgttgcccaatattatattacttggaatgaattatcctatgatcatcaaggtaaggaagtcttgactcctaaggcttgggacagaaatgggcaggatttaacggctcactttaccactagtattcctttaaaagggaatgttcgtaatctctctgtcaaaattagagagtgtaccgggcttgcctgggaatggtggcgtacggtttatgaaaaaaccgatttgccactagtgcgtaagcggacgatttctatttggggaacaactctctatcctcaggtagaagataaggtagaaaatgac

SEQ ID NO: 12 - DNA, 1566 bp, Helicobacter pylori
  source 1..1566: mol_type = genomic DNA
atggggcaggcattttttaaaaaaattgttggctgtttctgtcttggttatttatttttatctagcgcaatagaagcagcagcacttgacattaagaattttaatcgtggtagggtgaaagtggtgaataagaagattgcttatttgggagatgaaaaacctattacgatttggacttcattagacaatgttaccgtgatccaacttgaaaaagatgaaactatttcttacatcacaacaggtttcaataaaggttggagtattgtgcctaattctaatcatatattcattcaacctaaatcggtaaaaagtaatctcatgtttgaaaaagaagcagtgaattttgccctaatgacaagagattaccaagaatttttaaaaacaaaaaaacttatcgtagatgcgcctgaccctaaagaattagaagaacaaaaaaaagctctagaaaaagaaaaagaagctaaagaacaagcgcaaaaagcacaaaaagataaaagagaaaaaagaaaagaagagcgtgcaaaaaatagagccaatttagaaaatctcactaacgctatgagtaacccacaaaatttgagcaataacaaaaatcttagcgaatttatcaagcaacaacgagaaaatgaattagaccaaatggaacgactagaggacatgcaagaacaggctcaagctaatgcgctcaaacaaattgaagaactcaacaagaaacaagctgaagagacaatcaagcaaagagccaaagataaaatcaatattaagacagataagcctcaaaaaagccctgaggataactccatagaattatctcctagcgatagcgcttggagaactaatcttgttgtgcggactaataaagccttgtatcaattcattttgagaatagctcaaaaagacaattttgcttcagcgtatctaacagtcaaattagaatacccacaaagacacgaagtctctagcgttattgaagaggagttaaaaaagagagaagaagcaaagaggcagaaagaattgattaagcaagaaaatcttaacaccacagcctacatcaatagagtgatgatggcgagcaatgaacagattatcaacaaagaaaaaataagagaagaaaagcaaaaaattatcttagatcaagcaaaggcgctagagactcaatatgtgcataatgccttaaaaagaaaccccgtgcctagaaactacaactactaccaagcgcctgaaaaacgctctaaacatattatgccctctgaaatttttgatgatggcacattcacttattttggtttcaaaaacatcactctccaacctgctatttttgtggttcaacctgatgggaaattgagcatgactgatgccgccattgatcctaacatgaccaattcaggattgagatggtatagagttaatgaaattgcagaaaaatttaagctcattaaagacaaagcccttgtaacagtaatcaataaaggctatgggaaaaatccattgacaaaaaattacaatatcaaaaactatggtgaattggagcgtgtgattaaaaagctccctcttgtcagagataaa

SEQ ID NO: 13 - DNA, 585 bp, Influenza A virus
  source 1..585: mol_type = genomic DNA
atgcagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctca

SEQ ID NO: 14 - DNA, 813 bp, Helicobacter pylori
  source 1..813: mol_type = genomic DNA
atgaatgcattcaagcgtattattagtgtgggggtaattgctttaggtttgtttaaccttttagacgccaaacaccacaaagagaaaaaagaaaaccacaaaatcactcgtgagcttaaagtgggcgctaaccccgttccgcatgcgcaaatcttgcaatcagtcgtggacgatttgaaagagaaagggatcaaattagtgatcgtatcttttacggattatgtgttgcctaatttagcgctcaatgacggctctttggatgcgaattacttccagcaccgcccttatttggatcggtttaatttggacaggaaaatgcaccttgttggtttggccaatatccatgtggagcctttaagattttattctcaaaaaatcacagatattaaaaatcttaaaaagggttcagtgattgctgtgccaaatgatccggccaatcaaggtagggcgttgattttactccataaacaaggccttatcgccctcaaagacccaagcaatctatacgctacggagtttgatattgtcaaaaatccttacaatatcaaaatcaagcctttagaagccgcgttattgcctaaggttttaggggatgtggatggggctatcataacagggaattatgccttgcaagcaaaactcaccggagctttattttcagaagataaggactcgccttatgccaatctaatagccgctcgtgaggataacgcgcaagatgaagccataaaaacattgattgaagccttacaaagtgaaaagaccaggaaattcattttggatacctataagggggcgattatcccggctttt

SEQ ID NO: 15 - DNA, 657 bp, Influenza A virus
  source 1..657: mol_type = genomic DNA
atggactccaatacaatgagctccttccaggtggattgctttctgtggcacatcaggaagcggttcgcagacaacggactgggcgacgccccatttctggacaggctccggagagatcagaagagcctgaaaggccgcgggaataccctgggcctcgacatcgagaccgccacactggtcgggaagcagatcgttgagtggattctgaaagaggaatctagtgagaccctgcgaatgaccattgcttcagtgcctacaagcagatacctgtccgacatgaccctggaggaaatgtcccgggattggttcatgctgatgcccagacagaaaatcattggccctctgtgcgtgagactcgatcaggctatcatggagaagaacattgtgctgaaagcaaatttctctgtcatctttaacaggctggagaccctgattctgctccgcgcctttacagaggaaggagctatcgttggcgagatttctccactgccgagtctcccaggccatacttatgaagacgtgaagaatgcagtgggagtcctgatcggtggtctcgagtggaacggtaataccgtgcgggtgagcgaaaacattcagcgattcgcctggcgtaactgtgatgagaatggcagacccagcctgccccctgaacagaaa

SEQ ID NO: 16 - DNA, 135 bp, Helicobacter pylori
  source 1..135: mol_type = genomic DNA
atgctaaaaaatatgatctttgtcttgcatgaagcgtttgagttttcctatatctttaatggaattttcactcatcaaaagcttgtgtgttctttgttttgccaaccatcgctcttagtggattgttgcatgcgc

SEQ ID NO: 17 - DNA, 2505 bp, Helicobacter pylori
  source 1..2505: mol_type = genomic DNA
atgactgaagattttcctaaaattctgcctttattggtagaagaagacacctttttatacccctttatgatagcccctatttttttgcaaaataacgccagcattaaggcggtggcttacgctaaaaacaacaaatcgttagtctttattgcatgccaaaaagacaaattaaacgataatgaagccccttattatgatgtgggggtgataggttcggtgatgcgtgaagccaacatgcctaatgggcgcgtaaaattgctctttaatggcatcgctaaggggcgtattttagagcctgctaaagaaaacgagcaaggctttttagaagctcaaataagccccattgaatatttagaatacgataaagaaaacattcaagcgatcgtggaagtgttaaaagaaaaggtgatcactctagccaatgtcagctcactcttccccccggatttgatcaaggctttagaagacaatgacgatcctaaccgcatcgctgatttaatcgcagcggccttgcatttaaaaaaagatcaagcgtattctctttttgccaacaacaacaccgaacagcgtttgttggatttgattgatattgtgatagaagagactaaaacccaaaaactccaaaaagaaatcaaatccaaagtccatcaaaaaatggagcaaaccaataaggaatatttcttaaaagagcagctcaaacaaatccaaaaagagcttggcacagacaaacagcgagatgaagatttaaaccaatactaccaaaaactagaaagcatcaagccttttttaaaagaagaagcgtttaaggagattaaaaagcaaattgaccggctgagccgaacccatgcggacagctcggatagcgcgactttacaaaattatattgaaaccatgctggatgtgccttttgggcaatacgggaaaaaagcgcttgacattaagcatgtgagagaacaactagacaaggatcattattccttaaaaaggcctaaagagcgcattgtagaatactttgccaccatgcagcttttagaaatgcgccgcaagaaaaagccagaaaaaaaagacaaaactaaaggcacgattttatgcttttatgggcctcctggcgtgggtaaaacaagcttagctaattccatcgctaaagcgatagagcgccctttagttcggatcgctttagggggattagaagatgtgaatgaattaagagggcacagacgcacttatataggctcaatgcctgggcgcattgtccaagggcttattgaagctaaaaagatgaatccggtcatggttttagatgaaattgataaggtggataggagcgttaggggcgatccagcgagcgctttattagagatcttagaccctgagcaaaataccgcttttagggatcattatgcgaattttagcattgatttgtcgcaagtgatttttatcgctaccgctaacaatattgacagaatcccggcccctttaagagacagaatggaatttatcagcgtgtccagctacacgcctaatgaaaaagaagaaatcgctaaaaattatcttattccccaagaattagaaaagcacgccttaaagcctagcgaagttgagattagccatgagtgtttgaaactcattattgaaaaatacaccagagaagcgggcgttagggatttacgaagacagatcgcaacgattatgcgtaaagtggctttaaaatacctagaagataacccgcaccaaaaagggcgaaccaaaaaaggcaaaaatgaaaaaagcgaagatcaaaaaagcgaagatcaaaaaagcgaaaatcaaaaaagcgagaacaaagatttctgcgtctctatcacgcctaacaaccttaaagagtatttagagcgcatggtgtttgaaattgaccccatagatgaagaaaataaaatcggtatcgtcaatggtttggcatggactccagtgggcggtgatgtgcttaaaattgaagtgctcaagattagaggcaagggagaattgaaactcaccgggagtttaggcgatgtgatgaaagaatccgccattattgccttttctgttgtcaaagtcttattggataacgaaaccttaaaagtgcctaaaatccctagcgagaccgatgcagagggcaagaaaaagaaaaaagtgctgaaagtttataacgcttatgatttgcatttgcatgtccctgagggggctacgcctaaagacggcccgagcgctgggatcgctatggcaagcgtgatggcgagcattttgtgcgatagggctacaagaagcgaagtggcaatgacgggcgaattgactttgagcggggaagttttacccataggagggttgaaagaaaagttgatcgctgcttttaaagccggcattaaaaccgctctcattcctgtcaaaaattacgaaagggatttagacgagatccctgctgaagtgcgagaaaatttaaacatcgttgcggtgaaaaacatcgctgaagtgttagaaaaaactttgctt

SEQ ID NO: 18 - DNA, 1095 bp, Campylobacter jejuni
  source 1..1095: mol_type = genomic DNA
atgtttggaagtaaaataaaccattctgatcttcaaaaacttgaagaagaaaataaaaatcttacacataaaattgaaaaatttcaatcggaaaatttagaattaaaaaataaaattacatcattagaacaagcggctttagagtctaaacttaaaacagatcttttaaatgttttattaactggggttttaaaaaatattactattattcaaggcgatatgttagaaaatgtaaataaggcagaagttatctctagctattctaaaacttctcttgctgaaatggatgaacttaaccatattgctaattctatcaatgcctctttgggaaatattacagaatcagccaataaaacaagagatgttgcaggaacattgcatagaagtgtagatgaaattactaatgtaattaatctaattaaagatgtttccgatcaaaccaatcttttagctcttaacgctgccatagaagctgcacgcgcaggtgaacatgggcgtggttttgctgttgttgccgatgaagtaagaaaactagctgaaaaaacacaaaaagcaaccacagaagttgaaatgaacatcaaccttctaaagcaaaatgcaaatgaaatgtatactcaaagtgagcaagttgaaaaaatttctatagattctaatgctcatattatgagtttttccgaaaaatttactcacttagtaaatgaagcgcattctactaattcaaatgcagttggtatcgcttcagaagctttcgtatctttagcaaaactagatcatatagcttttaaattaaatggttataaagaaattttttccaaatcaggaaagcaattagccgatcatacatcttgtcgcttaggaaaatggcttgctagcacaggaaaagaaagatttgggcaaaataaaagcttcttaaaaattaacgagccacatgaaaaagttcatgaaaatatgaataatgctataacaatagcaaatacagaagatattagcaaagatattactcaacacagcataattaataaatgcgaagttgctgaaaatgcatcgcttgatcttttcaatgtatttaaagaaatgcttgatgagtcagatcat

SEQ ID NO: 19 - DNA, 621 bp, Streptococcus pyogenes
  source 1..621: mol_type = genomic DNA
atgaagaagggcctgctggtgaccacaggcctggcctgcctgggcctgctgaccgcatgtagcacacaggacaacatggccaagaaggagatcacccaggataagatgagcatggccgccaagaagaaggacaagatgtctacaagcaaggataagtctatgatggccgacaagagcagcgataagaagatgaccaatgacggccctatggcccctgatttcgagctgaagggcatcgacggcaagacataccggctgtccgagttcaagggcaagaaggtgtatctgaagttttgggcctcctggtgctctatctgtctgtccaccctggccgacacagaggatctggccaagatgtctgacaaggattacgtggtgctgaccgtggtgagccctggacaccagggagagaagtccgaggccgatttcaagaagtggtttcagggcacagactataaggatctgccagtgctgctggaccccgatggcaagctgctggaggcctacggcgtgcggagctatcccaccgaggtgtttatcggcagcgacggcgtgctggccaagaagcacatcggctacgccaagaagagcgacatcaagaagaccctgaagggcatccac

SEQ ID NO: 20 - DNA, 1440 bp, Human herpesvirus 4
  source 1..1440: mol_type = genomic DNA
atggttccttctcagagactctcccgaactagcagcatttcctccaacgaggatcccgcagagagccacattctggaactcgaagcggtctcagacaccaacacagactgcgatatggaccccatggagggcagcgaagaacactccacagatggagagatttcatcctcagaggaggaggatgaagatccaactccggcccacgccatacctgcacagccctccagcgtggtcataacccctacctcggcatcgtttgtgattcccagaaagaagtgggacctacaggacaagacagtcacattgcatcgctcacccctgtgcagggacgaggacgagaaggaggagactggcaactcctcttacaccagaggccacaaaaggcgacgcggagaggtccatggctgcaccgatgaaagttatggcaagcgccgacacctgcccccgggagcaagaatgcccagagccccaagggcccccaggatgcctagagcaccgaggtctccaagagctccccgaagcaacagagcaaccagaggtccccggtcagaatctagaggggccggcaggagcacaaggaagcaggcgaggcaagaacgcagccagaggcccctgcccaacaaaccgtggtttgacatgagtctggttaagcctgtctccaagattacatttgtcaccttgcccagccccctggcctctctgaccctagagcccatccaagacccgttcctacagtcgatgctggcggtggccgcccatccagagattggagcctggcagaaagtgcaacccagacacgagctgcgcaggagctacaagacgctacgtgagtttttcaccaagtcaaccaacaaggacacatggctggatgcacgcatgcaggcgatccagaacgcggggctctgcaccctggtagccatgctagaagagaccatcttttggctccaggagatcacctaccacggcgacctgcccctagctcccgcggaagacatcctcctggcctgcgccatgagtctcagcaaggtgatcctgaccaagctcaaagagctggcaccctgcttccttcctaacacgcgagactacaactttgtgaagcaactcttctacatcacctgtgccacggcccgtcaaaacaaggtggtggagaccctgagcagctcatatgtgaagcagcccctctgtctcttggcagcatatgcggcagtagccccagcctacattaacgcccactgcagacggagacacgatgaagttgaattcctgggccactacatcaagaattacaaccctggcacgctaagctcccttttgacagaggccgtggagactcacacacgtgactgccgaagtgcatcctgcagccgacttgtcagggccattctctccccgggcactgggtcactcggactgttttttgttcctggattaaatcaataa

SEQ ID NO: 21 - DNA, 2079 bp, Streptococcus pneumoniae
  source 1..2079: mol_type = genomic DNA
atgtttgcatcaaaaagcgaaagaaaagtacattattcaattcgtaaatttagtgttggagtagctagtgtagttgttgccagtcttgttatgggaagtgtggttcatgcgacagagaacgagggagctacccaagtacccacttcttctaatagggcaaatgaaagtcaggcagaacaaggagaacaacctaaaaaactcgattcagaacgagataaggcaaggaaagaggtcgaggaatatgtaaaaaaaatagtgggtgagagctatgcaaaatcaactaaaaagcgacatacaattactgtagctctagttaacgagttgaacaacattaagaacgagtatttgaataaaatagttgaatcaacctcagaaagccaactacagatactgatgatggagagtcgatcaaaagtagatgaagctgtgtctaagtttgaaaaggactcatcttcttcgtcaagttcagactcttccactaaaccggaagcttcagatacagcgaagccaaacaagccgacagaaccaggagaaaaggtagcagaagctaagaagaaggttgaagaagctgagaaaaaagccaaggatcaaaaagaagaagatcgtcgtaactacccaaccattacttacaaaacgcttgaacttgaaattgctgagtccgatgtggaagttaaaaaagcggagcttgaactagtaaaagtgaaagctaacgaacctcgagacgagcaaaaaattaagcaagcagaagcggaagttgagagtaaacaagctgaggctacaaggttaaaaaaaatcaagacagatcgtgaagaagcagaagaagaagctaaacgaagagcagatgctaaagagcaaggtaaaccaaaggggcgggcaaaacgaggagttcctggagagctagcaacacctgataaaaaagaaaatgatgcgaagtcttcagattctagcgtaggtgaagaaactcttccaagcccatccctgaaaccagaaaaaaaggtagcagaagctgagaagaaggttgaagaagctaagaaaaaagccgaggatcaaaaagaagaagatcgccgtaactacccaaccaatacttacaaaacgcttgaacttgaaattgctgagtccgatgtggaagttaaaaaagcggagcttgaactagtaaaagaggaagctaaggaacctcgaaacgaggaaaaagttaagcaagcaaaagcggaagttgagagtaaaaaagctgaggctacaaggttagaaaaaatcaagacagatcgtaaaaaagcagaagaagaagctaaacgaaaagcagcagaagaagataaagttaaagaaaaaccagctgaacaaccacaaccagcgccggctccaaaagcagaaaaaccagctccagctccaaaaccagagaatccagctgaacaaccaaaagcagaaaaaccagctgatcaacaagctgaagaagactatgctcgtagatcagaagaagaatataatcgcttgactcaacagcaaccgccaaaaactgaaaaaccagcacaaccatctactccaaaaacaggctggaaacaagaaaacggtatgtggtacttctacaatactgatggttcaatggcgacaggatggctccaaaacaatggctcatggtactacctcaacagcaatggcgctatggcgacaggatggctccaaaacaatggttcatggtactatctaaacgctaatggttcaatggcaacaggatggctccaaaacaatggttcatggtactacctaaacgctaatggttcaatggcgacaggatggctccaatacaatggctcatggtactacctaaacgctaatggttcaatggcgacaggatggctccaatacaatggctcatggtactacctaaacgctaatggtgatatggcgacaggttgggtgaaagatggagatacctggtactatcttgaagcatcaggtgctatgaaagcaagccaatggttcaaagtatcagataaatggtactatgtcaatggctcaggtgcccttgcagtcaacacaactgtagatggctatggagtcaatgccaatggtgaatgggtaaac

SEQ ID NO: 22 - DNA, 1053 bp, Streptococcus gallolyticus; identical to SEQ ID NO: 1
  source 1..1053: mol_type = genomic DNA

SEQ ID NO: 23 - DNA, 420 bp, Adenovirus
  source 1..420: mol_type = genomic DNA
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtt

SEQ ID NO: 24 - DNA, 1740 bp, Human herpesvirus 5
  source 1..1740: mol_type = genomic DNA
atggagtcctctgccaagagaaagatggaccctgacaaccctgacgagggcccttcctccaaggtgccacggcccgagacacccgtgtccaaggccacgacgttcctgcagactatgttaaggaaggaggttaacagtcagctgaacctgggagacccgctgttcccagaattggccgaagaatctctcaaaacctttgaacaagtgaccgaggattgcaacgagaaccccgaaaaagatgtcctggcagaactcggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcttctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcttgcaaaaccatgcaggtgaacaacaagggcatccagatcatctacacccgcaatcatgaggtgaagagtgaggtggatgcggtgcggtgtcgcctgggcaccatgtgcaacctggccctctccactcccttcctcatggagcacaccatgcctgtgacacacccacccgaagtggcgcagcgcacggccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagttccccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 25 - DNA, 711 bp, Pseudomonas aeruginosa; identical to SEQ ID NO: 2
  source 1..711: mol_type = genomic DNA

SEQ ID NO: 26 - DNA, 867 bp, Human rhinovirus A1
  source 1..867: mol_type = genomic DNA
atgaaccccgtggagaattacatcgatgaggtgctgaacgaagtcctcgtggtccctaatattaaggagtctcaccataccacatctaacagtgcaccactgctcgacgccgctgaaactggccacacctccaatgtgcagcctgaggatgccatcgaaaccagatatgtgattacatctcagactagagacgagatgtcaatcgaaagcttcctgggaaggagcggttgcgtgcacatctcccgcattaaggtcgactacacagattataacgggcaggatatcaatttcaccaagtggaaaattacactgcaggagatggcccagatccggagaaaattcgaactgttcacctacgtgcgatttgacagtgagatcacactggtgccatgtattgctggcagaggggacgatatcggacacattgtcatgcagtacatgtatgttccccctggtgcacccatccctagcaaaaggaacgatttctcctggcagtctggcaccaatatgtcaatcttctggcagcatgggcagccattcccgcgctttagcctgcccttcctgagtatcgcatcagcctactatatgttttacgatggctatgacggggataacaccagctccaagtacggaagcgttgtgactaatgacatgggtaccatctgctctaggattgtgacagagaagcagaaacacagtgtggtcatcactacccacatctaccataaggctaaacatactaaagcatggtgtccacgaccaccgcgtgctgttccctacacacactcccatgtcactaactatatgcccgaaactggcgacgtgacaactgccatcgtgaggcgcaataccattaccacagctttg

SEQ ID NO: 27 - DNA, 1161 bp, Human adenovirus 41
  source 1..1161: mol_type = genomic DNA
atgaagcggaccagaatcgaggacgatttcaatccagtgtacccgtatgacacctttagtacaccctcaattccttacgtggcaccacctttcgtcagctccgacggactgcaggaaaagccaccgggcgtgctggccctgaagtataccgatcctatcaccacaaacgccaagcacgagctgaccctgaagctgggcagtaatattacactggaaaacgggctgctctctgccactgtcccaaccgtgagcccccctctgaccaatagcaacaattccctcggcctggcaacatccgccccaatcgccgtgagcgccaacagtctgaccctggcaactgcagctccactgaccgtgagcaacaatcagctgtctatcaatgctggcagggggctggtcattactaacaatgctgttgcagtgaaccccactggcgctctggggttcaacaataccggcgccctgcagctgaatgcagcaggtggtatgcgagtggatggtgcaaacctgattctgcatgttgcctacccttttgaggctgtgaatcagctgaccctgcgtctcgagaacgggctggaagtcaccagcggaggcaagctgaacgtgaaactcggaagtggtctgcagtttgactcaaacggacgcatcgctatttcaaacagcaataggacacgctcagtgcccagcctgactaccatctggtccatttctcccacacctaattgctctatctatgagactcaggatgctaacctgttcctgtgcctgaccaagaacggcgcacacgtgctggggacaatcactattaagggcctgaagggcgccctgcgagaaatgcatgacaacgctctctccctgaaactcccctttgataaccagggcaacctgctgaactgcgccctggagtctagtacctggagataccaggaaacagacgccgtggcttcaaatgcactgactttcatgcctaacagcaccgtctatccaaggaataagacagctgatcccgggaacatgctgatccagattagccctaacatcactttttccgtggtctacaatgagattaactccggatatgcattcacctttaagtggtctgccgaaccaggcaagccgttccacccaccgacagccgtgttttgttacatcactgagcag

SEQ ID NO: 28 - DNA, 1380 bp, Streptococcus gallolyticus
  source 1..1380: mol_type = genomic DNA
atgaaaaaaagaattttatcagctgttcttgtcagtggtgtaacacttggaacggcagccgctacagtaaatgcggatgattatgatacacaaatcgctgcacaagatgcggttatcagtaatcttacatctgagcaagcagctgctcaaagtcaagttgatgctcttcaagaacaagtgacttcacttcaatcacaacaagatgaattggaagcacaaaatgctcaactcgaagcagaatctcaaaaattgagcgaagaaattcaagctctttcaagcaaaattgttgctcgtaatgaatctttgaaaaaacaagcacgcagtgctcaaaaaactaacacagctacaagctacattaatactatcttgaattctaaatcaatttcagatgctattaatcgtgtagctgctgttcgtgaagttgtttctgcaaacgaaaaaatgttggaacaacaagaagcagataaagctgctatcgaacaaaaacaagctgaaaaccaagaagctatcaatacagtagctgctaataaagcaactattgaacaaaaccaagcagctttggcaactcaacaagctgaattggaagcagctcaattgaacttgtcagctcaattggcaactgcagaagatgaaaaagcaagcttagtagctcaaaaagaagctgctgaacaagcagcggctgaagcagctgcagcacaagctgccgcggaagcacaagcacaagctgaggcagaagcgcaagctgcatctgtagcacaagcacaagaatctgttgaaaatggtacagctactgttgatactacaacagatacatcatcacaagattcaactactgcatctacagatactgcagcagcaactgaagatacttcttcaactcaacaagctgctacagttactccaactgctacaacaactacatcaagttcatcaagtagctcatcagcttcaagttcatcatcaagtagttcatcagcttctacaagctcaactgcttcaacatcaacttcatcaagcagttcaagtagctcatcttcaagctcagtgaacacttaccctgttggtcaatgtacttggggtgttaaatcacttgcttcatgggttggaaacaactggggtaatgcaaatcaatggattgctagcgcacaagctgcaggtcattctgtaggtactacaccacaagttggtgcggttgctgtatggccttatgatggtggtggatatggacacgttgcatatgtaactgcagttcaaagttctacaagtatccaagttatggaagctaattatgctggtaactcatctattggtaactaccgtggatggtttgacccaacttcatcaacttggggtggcggtactgtttactacatctatcaa

SEQ ID NO: 29 - DNA, 1713 bp, Human herpesvirus 4; identical to SEQ ID NO: 4
  source 1..1713: mol_type = genomic DNA

SEQ ID NO: 30 - DNA, 2079 bp, Streptococcus pneumoniae; identical to SEQ ID NO: 21
  source 1..2079: mol_type = genomic DNA

SEQ ID NO: 31 - DNA, 588 bp, Helicobacter pylori
  source 1..588: mol_type = genomic DNA
atggatattttagatttgaacaaagcgcaagcggtgcaacaaaatgaacaagaggtagaggataaagagcgagagtctaaagagccggtggttttagaagatttgagcgctttagcgtggcttgaattagaagagtttagccgcctttcagggcttcctaaagaaaggattttggaattagtgaatcttggtaaaatcaagagcaaaataagcagcaacaagcttttaattgatgcgagcagcgggacaaacgctttaatcaaaaaggtagaaaatagtttgatttctatggatatgaacgggcgttctttagaacctgtgtttgtggaaaagaccattaacacgattttaaacttgcatgataaggtcattggcgctaaagatgaaacgatttcagcctttaaaaatgaaaacatgtttttaaaagacgctttaatctctatgcaagaagtctatgaagaagataaaaaaaccattgatcttttgcgcgatgaactcaatcaagcgagagaagaaattgaatttatgaagaggaaataccgcttgatgtgggggaaagtcgctgacatgagcagcgtgaataaaaag

SEQ ID NO: 32 - DNA, 2100 bp, Helicobacter pylori
  source 1..2100: mol_type = genomic DNA
atgctaagattcgttagtaaaacgatttgcttgtctttaatcggcttgttcaaccctttagaagcctttcaaaaacaccaaaaagacggcttttttatagaagctgggtttgaaactgggttattagaaggaacgcaaactaaagaagaagtcataaccacccaaaaaatctatgaaaaccccctaacccacccacaaactaaagaacagcctaaagaacaaaataaaagcgatacggccaccccacaaagcgcttacggaaaatactacataccccaaagcaccattttaaaaaatgcaacggctttattcaccacggacaagatagaaaatggcttaactttttattctcaaaaccctgtgtatgcgaatatggttaatgggagcgtaaccatacaaaactttctgccttataatttaaacaatgttgaactgagttttaaagacgctcaaggcaaggtggtcaatttaggcgtgatagagaccatccctaaacaatctcaaattaccttgcctgcaagcttgtttaatgattcagaatttgaacaagctgatagctttaattaccaacaacttcaagccactgccacacaattttctgacgctaacacgcaaagtttgtttcaaaagctcagcaagatcacaaccaatgtaacaatgagttatgaaaacgccgataccaacaattttaaaggtaattgccatgattgtgtgtcagatttcaccccacaaaccgcagaagaattgaccaatttaatgctagatatgattgcggtgtttgactctaaatcgtgggaagaagccgttttaaacgctcctttccaattttctaacagctcatcagagtgcggctctgactttcctaagtgcgtgaatcctttcaataacgggcgtgtcgctcccatctatgaaaaatacgtgctaaccccacaatccgttatagatgcgtttagaagaacgatcaatcttgaagtgaatatcctaaaatcagggtttgtagggctagggtatgaacttgatgataatgatggtaatctggggatagaagcttctgccttaaatcctgaaaaattgtttggtaaaactttgaacaaagttgatattgtggaattaagagacattatccatgaatttagccacactaaaggctatacgcataatgggaacatgacttatcaaagagtgcgcttgtgtcaagaaaacggcggagccatacaagaatgtgagggtgggaaagaagagttagtcaatggaaaagaagaactaaaatttacaaatgggaaagaagtgaaagatcaggatggttacacctatgatgtatgttctttttataaggacaaccaccaagtctatacagcgagcaattaccccaattccatttatacgaattgcgctcgagtccctgctgggcttataggggttaccaccgctgtctggcaacagctcatcaatcaaaacgctctgcccattaatttcgctaatctaaatagcccaaccaaccacttaaacgccgggttgaacgcacaaaattttgcaacctctatagtcagcgcgatcgcgcaaaatttttccaccacttccaccaccacttaccgctcttcaagtaagaattttagaagccctattttaggggttaatgttaaaataggctaccaacattatttcaatgactacatagggttagcctattacggcattatcaaatacaattacgccaaaactaacgatgaaaaaatccagcaattaagctatggtgggggaatggatgtgttgtttgatttcatcaccacttacgctaacaaaaagcaagacaacccaactaaaaaagcttttgcttcctcttttggggtgtttggggggttaaggggcttatacaatagctattatgtcttcaaccaagtcaaaggaagcggtaatttagatatagttactgggtttaattaccgctacaagcattctaaatattctgtaggcattagcgttcctttaatccaaagcggtattaaaatcgcttctaataatggcatctatgcgaactccgttgttttgaatgaagggggcagtcattttaaagtgttttttaattacgggtggattttt

SEQ ID NO: 33 - DNA, 795 bp, Human mastadenovirus A
  source 1..795: mol_type = genomic DNA
atggaggacatcaacttcagctccctggcaccaagacacggcaccaggccctacatggggacatggaacgaaattggaactagccagctgaatggcggggctttcaactggaattcaatctggagcggcctgaagaacttcggttctacaattaagacttacgggaccaaagcctggaatagtcagaccggacagatgctgcgcgacaagctgaaggatcagaactttcagcagaaagtggtcgacggcctggctagtggcatcaacggggttgtggatattgctaatcaggcagtgcagaagaaaatcgcaaatcgcctggagccacgaccggacgaagtcatggttgaggaaaagctgccccctctcgagaccgtgccaggtagcgtcccgacaaagggcgaaaaacgcccccgacctgatgcagaggaaacactggtgactcataccacagagccaccgtcatacgaggaagccatcaagcagggtgccgctctgagccctactacctacccaatgactaagcccatcctgcctatggccaccagggtgtatgggaaaaacgagaatgtgcctatgacactggagctcccccctctgccagaaccgactatcgcagatccagtgggtagcgtgccagtcgcctccgttcctgtggcttccaccgtgtctagacccgctgtcaggcctgtcgcagttgcctccctgcgtaacccccggtctagtaattggcagagcaccctgaactcaattgttggactgggtgtcaagagcctgaagcggagaaggtgctat

SEQ ID NO: 34 - DNA, 447 bp, Human mastadenovirus C
  source 1..447: mol_type = genomic DNA
atgcgcgcggatcgcgaagaactggatctgccgccgccgattggcggcgtggcgattgatgtggtgaaagtggaagtgccggcgaccggccgcaccctggtgctggcgtttgtgaaaacctgcgcggtgctggcggcggtgcatggcctgtatattctgcatgaagtggatctgaccaccgcgcataaagaagcggaatgggaatttgaaccgctggcgtggcgcgtgtggctggtggtgttttattttggctgcctgagcctgaccgtgtggctgctggaaggcagctatggcggcagcgatcatcatgcggcgcgcgcgcagagcccggatgtgcgcgcgcgccgcagcgaactggatgataacattgcgcagatgggcgcggtgcatggcctggaactgccgcgccgccaggtgcgccgcgaactgctgcaggtgtatctggcg

SEQ ID NO: 35 - DNA, 1809 bp, Mycobacterium tuberculosis
  source 1..1809: mol_type = genomic DNA
atgtcgtttgtaatcgccgctcccgagacgctggtgagggccgcttcggatttggcgaacatcgggtcgacgctgggggccgccaacgccgcggcattgggcccgacgacggaacttctggcagccggtgctgatgaggtgtcggcggccatcgcgtcgctgttcgccgcgcatggccaggcctatcaggcggtgagcgctcagatgtcggcgtttcatgcccagttcgttcaaacctttactgcgggcgctggcgcgtacgcgagcgctgaggccgccgccgcggcgccgttggagggcctgctcaacattgtgaacacgcccacccagctgttgctggggcgtccgctgatcggcaacggcgccaacggcgcaccggggaccgggcaagccggcggggccggcgggctgctgtacggcaacggcggggccggcgggtccggggcgcccggccaggccggcggcccaggtggagccgcggggctattcggcaacggcggggcgggtggggccgggggtgacggcccgggcaacggcgcggctggcggggccggcggggccggcgggctgctgttcggctccggcggcgccggtgggcccggcggagtcgggaacaccggcacaggcgggctcggcggcgacggcggggcggccgggctgttcggagccggtggcatcggcggggccggcgggccaggcttcaacggtggcgccggcggggccggcggtcgcagcgggctgttcgaggtgttggccgccggcggtgccggcggcaccggcgggttgagcgtcaacgggggcaccggtggcaccggcggcaccggcgggggcggcgggctgttctccaatggcggtgccggcggcgccggcgggttcggagtctccggctcggccggagggaacggcggcaccggaggcgacggcggcatcttcaccggcaacggcgggaccggcggcaccggcggaactggcaccggtaaccaactcgtgggcggtgagggcggggccggcggggcgggcggcaacgccggcatcctcttcggcgctggcggcattggcggcaccggcggaaccggtctcggcgcgcccgaccccggcggcaccggcggcaaaggaggcgtcggcggcatcggcggtgccggcgcactgttcggtcccggcggcgccgggggcaccggagggttcggcgcctccagtgcggaccaaatggccggcggcatcggcgggtccggtggcagcggcggggccgccaagctgatcggcgacggcggcgccggcggtaccggcggcgacagcgtccggggcgcggccggtagcggcggcaccggcggcaccggcgggttgatcggtgacggcggcgctggtggcgccggcgggactggcatcgaattcgggtcggtcgggggggccggcggggccggcggcaacgccgccgggctctcgggtgcgggcggggccggcggagccggcgggttcggcgagaccgccggcgacggtggggccggcggcaacgccggcctgctcaacggcgatggcggcgccggcggcgccggcggactcggcatcgccggcgacggggggaacggcggcaagggtggcaaggccgggatggtcggcaacggcggcgacggcggcgccggcggcgcaagcgtggtggcaaacggcggtgtcggcggctccggcggcaacgccaccctgatcggcaatggaggcaacggcggaaacggcggagtcggcagtgcgccgggtaagggtggcgccggcggcaccgccgggctgctcggcctgaacggatcacccgggttgtcg

SEQ ID NO: 36 - DNA, 879 bp, Streptococcus pyogenes
  source 1..879: mol_type = genomic DNA
atgctgaccttcggcggggcatcagccgttaaggctgaggaaaacgagaaagtgcgcgaacaggagaagctgatccagcagctcagcgaaaagctggtggagattaacgacctgcagaccctgaacggcgataaagaaagcatccagagcctggtggactacctgacccggagaggcaagctggaggaagagtggatggagtacctgaacagcgggattcagagaaagctgtttgtgggccccaaggggcctgcaggagaaaaaggcgagcagggccccactgggaagcagggagaaaggggcgagacaggtccagcaggtccccggggcgacaagggtgaaacaggcgataaaggggcacagggaccagtgggtcctgcaggcaaggacggtcagaacggaaaagatggcctgccaggcaaggacgggaaagatggacagaatggcaaggacggcctgccagggaaggacggaaaagatggtcaggatggaaaagacggcctgcctgggaaggatggaaaagacggacaggacgggaaggatggactgccaggcaaagatgggcagcccggaaagcccgctcctaaaacaccagaggtgccccagaaccccgacactgcacctcacacaccaaagactccgcgaatccctggacagtctaaagatgtcacaccagccccgcagaaccctagtaatcgtgggctgaacaagccacagactcagggaggtaatcagctggccaaaacccccgccgctcacgatacacatagacagctgcccgctaccggcgaaaccacaaatcctttctttacagcagccgctgtcgccattatgactaccgctggcgtggtcgctgttgcaaagaggcaggagaacaat

SEQ ID NO: 37 - DNA, 1107 bp, Helicobacter pylori
  source 1..1107: mol_type = genomic DNA
atgcaatttcaaaaagccttattacattcatcattctttttacctttatttttatctttttgtatcgctgaagaaaatggggcgtatgcgagcgtgggttttgaatattccattagtcatgccgttgaacacaataacccctttttaaatcaagaacgcatccaaatcatttctaacgctcaaaataaaatctttaaactccatcaagttaaaaatgaaatcacaagcatgcctaaaacctttgcatatatcaacaacgctttaaaaaacaactccaaattaacccccactgaaatgcaagccgaacaatactacctccaatccacctttcaaaacattgaaaaaatagtaatgcttagcggtggcgtttcatctaacccacaattagtccaagcgttggaaaaaatgcaagaacccattactaaccctttagaatttgaagaaaacttaagaaatttagaagtgcaatttgctcaatctcaaaaccgcatgctttcttctttatcttctcaaatcgctgccatttcaaattccttaaacgcgcttgatcctaactcttattctaaaaacatttcaagcatgtatggggtgagtttgagcgtaggttataagcatttctttaccaagaaaaaaaatcaagggttgcgctattacttgttttatgactatggttacactaattttggttttgtgggcaatggctttgatggtttaggcaaaatgaataaccatctctatgggcttgggatagactatctttataatttcattgataatgcaaaaaaacactctagcgtaggtttttatctgggttttgctttagcggggagttcgtgggtagggagtggtttgagcatgtgggtgagccaaacggattttatcaacaattacttgacgggctatcaagctaaaatgcacacgagttttttccagatccctttgaattttggggttcgtgtgaatgtcaataggcataatggctttgaaatgggcttgaaaatccctttagcgatgaattccttttatgaaacgcatggcaaagggctaaacacttccctctttttcaaacgccttgtcatgtttaacgtgagttacgtttatagtttt

SEQ ID NO: 38 - DNA, 1572 bp, Human parainfluenza virus 1
  source 1..1572: mol_type = genomic DNA
atggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatt

SEQ ID NO: 39 - DNA, 1263 bp, Human herpesvirus 4
  source 1..1263: mol_type = genomic DNA
atggtcgatgaacaagtggcggtggaacacggcaccgtcagccacacaatcagccgggaggaagatggcgtggtccacgagcggcgtgtcttggccagcggggagagggtggaagtcttttataaggctccggccccacgacctcgggagggacgggcctctaccttccacgacttcaccgtcccggcggctgctgccgtcccagggccggaacctgagcctgagccacacccacctatgccaatccatgccaatgggggaggagagaccaagaccaatacccaggatcagaatcaaaatcagaccacccggacccggaccaatgccaaagctgaagaacggactgcggagatggatgacaccatggcctcgtcaggagggcagagaggggcaccaatttccgcggacctactctctctctcctcgttaactggtagaatggcagccatggccccctcctggatgaagagcgaggtatgcggtgagagaatgagattcaaggaagatgtctatgatggagaggcagagaccctagctgagcctccacgctgtttcatgctgagctttgttttcatctattactgctgctatttggcgttcctggccctactcgcctttggttttaacccactctttttgcccagctttatgccggtgggggccaaggtgcttcggggtaaggggcgtgattttggggtgcccctgtcttatgggtgtccgaccaatccattctgcaaggtttacacccttatcccggccgtggtcattaacaatgtgacttattaccccaacaacacggacagccatgggggtcatggtggatttgaggcggctgcccttcatgtagctgcactttttgagtctgggtgcccaaatctacaggctgtgactaataggaacaggacatttaacgtcaccagagccagtggccgagttgaaaggcgccttgtacaagatatgcagagggtcctggcgagtgctgtggtggtgatgcatcatcactgccactatgagacatattatgtctttgatggggtgggccccgagtttggtaccattcctacgccctgcttcaaggatgtgttggcctttaggccgtcattggtgaccaactgcaccgcgccgttaaagacatccgtcaagggtcctaactggtcaggggcagctggaggcatgaaacggaagcaatgtcgtgttgaccggctcacggaccgctcattccctgcatacctcgaggaggtcatgtatgtgatggttcagtag

SEQ ID NO: 40 - DNA, 798 bp, Streptococcus pneumoniae
  source 1..798: mol_type = genomic DNA
atgagaaaacaaattcatccacttattttatttagtttttttgggattatgatgttcattttaatcatcaatcgcccacttaatgacagccaatcgtttaaaacaaaatccaatatagcacagattgaagcacaggctttgaaacacttagacaaaccgattattgacctctctggctggcagcgtcctgaggaaatcaactacgatgccctctcacaaaatatttcaggtgctattgttcgtgttcacagtggtgctcaaacgacaaaagaaaatgatgcttcatttatcaatggaatagacaaggcctataagagtcatatcactgaacttcaaaagcggaatgtcccagttgctgtctacgcttatgtagctggaaaaagtgtccaagaaatggaaaaggctgccgaagttttctacaatgctgcctctccttacagccctagttactattggcttgacgtggaagacaaaaccatgtctaatatgaacgaaggtgtcgaaaactttcgagcaaaactagcatcactgggtgctaaaaacatcggcatctacgttggtgtctacttcatggaagaacacagtattgatacaggcaagtttacctctgtttggattccgtcctacggctcagactcaggatttttggagagcagtcctaaaactgatttagattatgatattcaccaatacacatctaaaggaaaaattgccggctttgaccacgatttagatatcaacgttatctctcccttaaaaaataaagaagaaacctttcgaaaactttttttaaaacct

SEQ ID NO: 41 - DNA, 1698 bp, Influenza A virus
  source 1..1698: mol_type = genomic DNA
atgaagaccatcattgcactgtcctatatcctgtgcctggtgttcgcccagaaactgcctggcaacgacaattctaccgctacactgtgcctcggccaccatgcagtgccaaatgggacaatcgtcaagactattaccaacgatcgcatcgaggttacaaatgccactgaactggtgcagaacagctccatcggcgagatttgcgactccccacaccagatcctggatggagaaaactgtactctgattgacgcactgctcggcgacccacagtgcgatgggttccagaataagaaatgggatctgtttgtggagcggagcaaggcttacagtaactgttacccctatgacgtgcctgattatgcaagtctgcgctcactcgtcgcctctagtgggacactggagttcaacaatgaaagctttaattgggccggagtgactcagaacggcaccagcagctcctgcatccgcggaagtaattctagtttcttttcacgactgaactggctcactcatctgaattccaagtaccccgccctgaacgtgaccatgcccaacaatgagcagttcgacaagctgtatatctggggcgtccaccatcctgggaccgacaaagatcagatctttctgtacgcccagtcaagcggaagaattaccgtctcaacaaagaggagccagcaggccgtgatccccaacattggcagccggcctagaatcagggacattccaagccgcatctccatctactggacaatcgtgaaaccgggcgatatcctgctgatcaacagcactggaaatctgattgctccccgtggttacttcaagattcggagcggcaaatcctctatcatgcggtccgacgcacctattgggaagtgcaaatctgagtgtattaccccaaacggaagtatcccaaatgataagccgtttcagaacgtcaatagaatcacatacggcgcttgtcccagatatgttaagcagagcaccctgaaactcgcaacagggatgagaaacgtgcctgagcgccagacccgaggcatcttcggggctattgcagggtttatcgagaatggatgggagggtatggtcgacggctggtacgggttccgccaccagaactcagagggtaggggtcaggcagctgacctgaagagcactcaggcagccatcgatcagattaacggcaagctgaaccgactgatcggaaagaccaatgaaaaattccatcagattgagaaggagttcagcgaggttgaaggccggatccaggatctggagaagtacgtggaagacaccaaaattgatctgtggagttataatgccgagctgctcgtggccctggaaaaccagcacacaatcgacctgactgattctgagatgaacaagctcttcgaaaaaacaaagaaacagctgagagagaacgcagaagacatgggaaacggttgtttcaagatctatcacaagtgcgataatgcctgtatcggatccattaggaacggtacttacgaccatgacgtgtaccgtgacgaggccctgaacaatcggttccagatcaagggagtcgagctgaagtctggttacaaagattggatcctgtggatttccttcgctatttcttgctttctgctgtgcgtggccctgctcggcttcatcatgtgggcttgccagaaaggcaacattaggtgcaatatctgtatt

SEQ ID NO: 42 - DNA, 345 bp, Human mastadenovirus C
  source 1..345: mol_type = genomic DNA
atgggcgaaacctggtttctgaccccgaacggccagagcagcccgggcagctggaacgcgcgcccgagcgcgggcccggcggcgcgcatgccgaccccgcgcaaccgctattttagccgcccgagcagcaccccgagcaaagtgtgcaccgcgccgcgcgcggcgccgccgccgcgcgcgagctgcgcgccgcgcgcgaccccgcgccgcggctggaccatgaccccgtggagcaacgcgacctggccgacccgccgcgcgaaaaccggcagcgcgagcgcgccggcgggcccggcgagcagcgcgccgtggccggcgcgcagcccggaacgcaccccgagcccg

SEQ ID NO: 43 - DNA, 2772 bp, Pseudomonas aeruginosa
  source 1..2772: mol_type = genomic DNA
gtgagcctgtccaccactgcatttccctcactgcaaggagagaacatgagtcgctcacccatccctcgccaccgagcgttgctggccggtttctgcctggctggcgcgctgtccgcccaggctgccacccaggaagaaatcctcgatgcggcactggtcagcggggattcctcgcaactgaccgacagccacctggtcgccctgcgcctgcagcagcaggtcgagcgcatccgccagacccgcacccagttgctcgacggtctctaccagaacctcagccaagcctatgatcctggcgccgccagcatgtgggtcctgccggccaacccggacaataccctgcccttcctcatcggcgacaaggggcgcgtgctcgccagcctgagcctggaggccggcggccgcgggctggcctatggcaccaacgtgctcacccagttgagcgggaccaatgccgcccacgcgccgttgctgaagcgggcggtgcagtggctggtgaacggcgacccgggcgcggccactgcgaaggacttcaaggtcagcgtggtcggggtggacaagaccgccgccctcaacggcctgaagagcgccggcctgcaaccggcggacgccgcctgcaacgcgctgaccgacgccagttgcgccagcaccagcaaattgctggtactgggcaacggcgccagcgccgctagcctgagcgccacggtgcgcgcacggctacaggccgggctgccgatcctcttcgtgcacaccaatggctggaaccagagcagcaccggccagcagatcctcgccggcctgggcctgcaggaaggcccctacggcggtaactactgggacaaggacagggtgccatccagccgcacccggacgcgttcggtcgagctgggcggcgcctatggccaggacccggcgctggtgcagcagatcgtcgacggtagctggcgcacggactacgactggtcgaaatgcacctcctacgtcggtcgcaccacctgcgacgacgtgccgggactgagcgacttcagcaagcgcgtcgatgtattgaaaggcgccctcgacgcctacaaccagaaggcgcagaacctcttcgccctgcccggcaccacctcgctgcggctctggctgctgtgggccgacgcggtgcggcagaacatccgctacccgatggacaaggccgccgacacggcgcgcttccaggaaaccttcgtcgccgacgccatcgtcggctacgtgcgcgaagccggcgcggcgcagaaggaactgggcagctacgccggccagcgccaacagtcgatgccggtatcgggctcggaggaaaccctgaccctcaccttgccgtcggcccagggcttcaccgccatcggccgcatggcggcgccgggcaagcgcctgagcatccgcatcgaagacgctggccaggccagcctggcggtcggcctgaacacccagcgcatcggctcgacgcggctgtggaatacccgccagtatgaccgtccgcgcttcctcaagagcccggacatcaagctccaggcaaaccagtcggtggcgctggtatcgccctacggcggcctgttgcaactggtctacagcggcgccacgccgggtcagacggtaaccgtcaaggtaaccggcgcggccagccagccgttcctcgacatccagccgggcgaggacagcagccaggcgattgccgacttcatccaggcgctggacgccgacaaggccgactggctggagatccgcagcggctcggtggaggtccacgccaaggtggagaaggtccgtggctcgatcgacaaggactatggcggcgacgtccagcgcttcatccgtgagttgaacgaggtgttcatcgacgacgcctacaccctggccggcttcgccattcccaaccaggcgaagactccggcaatccagcaggaatgcgcggcgcgcggctgggactgcgacagcgagaccctgcacaagctgcccggcacccagcacatcaacgtcgaccagtacgcccagtgcggcggcggttgcagcggcaacccctacgaccagacctggggcctgaacccgcgtggctggggcgaaagccatgagctgggccacaacctgcaagtgaatcgactgaaggtctacggcgggcgctcgggcgagatctccaaccagatattcccgttgcacaaggactggcgcgtgctccgcgagttcgggcagaacctcgacgatacgcgggtcaactaccgcaacgcctacaacctgatcgtcgccggacgcgccgaggccgatccgctggcaggcgtatacaagcgcctgtgggaagacccgggcacctacgcgctgaacggcgagcggatggccttctatacccagtgggtgcactactgggccgacctgaagaacgatccgctgcagggttgggacatctggaccctgctctacctgcaccagcgccaggtggacaagtccgactgggacgccaacaaggccgctctcggctacggcacctacgcccagcgcccgggaaacagcggcgatgccagcagcacggacggcaacgacaacctgttgctcggcctgtcctggctgacccagcgcgaccagcggccaaccttcgcgctatggggcatccgtacctcggcagcggcccaggcgcaggtggcggcctacggtttcgccgagcagccggcgttcttctatgccaacaaccggaccaacgaatacagcacggtgaagcttctggacatgagccaggggtcgccggcgtggccgtttccgtga

SEQ ID NO: 44 - DNA, 447 bp, Human mastadenovirus C; identical to SEQ ID NO: 34
  source 1..447: mol_type = genomic DNA

SEQ ID NO: 45 - DNA, 2079 bp, Streptococcus pneumoniae; identical to SEQ ID NO: 21
  source 1..2079: mol_type = genomic DNA

SEQ ID NO: 46 - DNA, 726 bp, Influenza C virus
  source 1..726: mol_type = genomic DNA
atggctcacgagatcctgattgccgagacagaagccttcctgaagaacgtggcacctgaaactcggaccgcaatcatttctgccatcactggcgggaagtcagcctgcaaaagcgccgctaagctgatcaagaatgagcacctgcccctcatgagtggcgaagctaccacaatgcatatcgtcatgcggtgtctgtaccccgagattaagccttggaagaaagctagcgacatgctgaacaaagcaaccagcagcctgaagaagagcgaggggagggacatccgcaagcagatgaaagcagccggcgatttcctgggggtggagtccatgatgaagatgcgagccttccgggacgatcagatcatggaaatggtggaggaagtctacgaccacccagacgattataccccggatatcaggattggcacaattactgcatggctgcgctgcaaaaacaagaaatccgagcggtatagatctaatgtctctgaaagtggacgaacagctctgaagatccatgaggtgcggaaagcttctaccgcaatgaatgaaatcgccggtattacaggactcggcgaggaagctctgagtctccagaggcagactgagtcactggccatcctgtgcaaccacacctttggcagcaatatcatgcgcccccatctggaaaaggccatcaagggtgttgagggtcgtgtgggagaaatgggtagaatggccatgaag

SEQ ID NO: 47 - DNA, 1677 bp, Human mastadenovirus D
  source 1..1677: mol_type = genomic DNA
atgcagcaggcaccagatccagctatccgtgccgctctgcagagtcagccatcagggctggcatccgacgattgggaagcagccatgcagagaattatggccctgaccacaaggaaccccgagtctttccgccagcagcctcaggccaatagactcagtgcaatcctggaagccgtggtcccttcaaggactaacccaacccacgagaaggttctggccatcgtgaacgctctggcagaaaacaaggccattcggcccgacgaggctggactggtctacaacgcactgctcgaacgagttggccggtacaactctactaatgtgcagagtaacctggatcgactggtcaccgacgtgcgggaggcagtggctcagcgagagcggttcaagaacgagggtctcggtagcctggtggccctgaacgcttttctggcaacacagccggctaatgtgcccaggggacaggacgattacacaaacttcatcagcgccctgcgcctgatggtgactgaagtccctcagtcagaggtctaccagagcggcccagattatttctttcagacaagccggcaggggctgcagactgtgaacctgtcccaggccttcaaaaatctgcgcggactctggggtgtccaggctcctgttggcgatcggtctactgtgagctccctgctcacccccaacagtagactgctcctgctcctgatcgccccttttaccgactcaggcagcgtgaacaggaatagctacctggggcacctgctgacactgtatcgcgaggctatcggacaggcacaggtggacgaacagaccttccaggagattacatccgtctctcgggctctgggtcagaatgacacagatagcctccgcgccaccctgaacttcctgctgaccaatcgacagcagaagatccctgcccagtacgctctgtccgcagaggaagagcggattctgagatatgtgcagcagtctgtcggactgttcctgatgcaggaaggtgcaaccccaagtgctgcactggatatgacagcccgtaacatggagccatctatgtacgccgctaaccgacccttcatcaacaagctcatggattacctgcacagagcagccgtgatgaatacagactacttcaccaacgccattctgaatccccattggctgccccctccaggattctacactggcgagtatgacatgcctgatccaaacgacggctttctgtgggacgatgtcgacagcgtggtgttcagccctacctttcagaagcgtcaggaagcaccaccatccgagggtgctgtggggagatctccgtttcccagcctgggctccctccactctctgccaggcagtgttaactccggtagggtgagccgtccaagactcctgggtgaagatgagtacctgaacgacagcctcctgcagcctccacgcgccaaaaatgcaatggccaacaatggaatcgaatccctcgtggataagctgaatcggtggaaaacctatgcccaggatcacagagacgctcctgcaccacggagacagaggcatgacaggcagcgtggtctggtgtgggacgatgaggattcagctgacgattctagcgtgctggacctgggtggtagcggaggtgttaatcccttcgcccacctgcagccaaagctggggaggcgcatgttt

SEQ ID NO: 48 - DNA, 2100 bp, Helicobacter pylori; identical to SEQ ID NO: 32
  source 1..2100: mol_type = genomic DNA

SEQ ID NO: 49 - DNA, 1689 bp, Mycobacterium tuberculosis
  source 1..1689: mol_type = genomic DNA
atgtaccggtaccaagtccgagtgcagcagcgcaggtcggagatgaacagatgggttgcgacacggtcacggaggcatacctatcagtggatcactgatcacaagagtcccagagatcactatcgtcacatcagcgaactgcggacgtcaattgcaacgtccagcccgggaaggtgtgacatgtcgccgattcctcgaatcgtgtcggtatcgctggcgtgggcggccgctattggactcatggttccaataggcctggcgccaccagcgatggcggcgccgtgcagcggagacgccgcgaacgctccgccgccaccgagcgccattgtgaccgaccccggggccacggcgctcggcccggtacgacctggacacggtccgataccgaccggacgcaagcctcgcggtgccaacgaccgtgcaccgctacccaagctgggtccactgatatccgcattgctcaacccgggtgctagaaatgccgcgccgctacaacagcaagcgctagtccccagggccaatcctgggcccaatccggcacccaatccgccagccacgggcccacaacccccgaatgccactcagctaactccgaacccagccccagcacccgaccctgctccggcggcggcgccggatcctggtgccacgctcgccggggctaccacatccctcgccgagtgggtgaccggaccggacagccccaacaagaccctggaacgcttcggcatctccgggaccgacctcggaattccttgggataacggcgatcccgccaaccgtcaggtgctcatgatcttcggcgacacattcggctactgcgctgtcgatggccatcaatggcgatacaacacgctgttccgcagccaagaccgcgacctgggcaacggagttcatgtgacatccggtgacgcttccaacagatattccggctcaccggtacgccaaccgggcttctccaaacagctcatcaacagcatcaaatgggcgcgcgacgagacggggatcattccgaccgcgggcatcgccgtcggcaaaacccaatacgtcaacttcatgtccatcaggaattggggccgtgatggggaatggacgacgaactactcgggcatcgcggtgtccaaggacaatggtcagacctggggggtcttcccgggcaccatccgcgcgtccggaccggacagcggcggaaaagccaggttcgttccgggaaatgagaacttccagatgggggcgtacctcaagtccaacgacggttacctctactcgttcgggaccccgcccgggcgaggcggttcggcatatctggcacgagttccgcagcgctttgtgcccgacctcaccaagtaccagtactggaacggcgactcgaactcctgggttccaaacaagccggacgcggcaacacccgttattccgggcccggtgggcgaaatgtccgtccaatacaacacctacctcaagcaatacctggcgctctacaccaacggtatgaacgacgtggtggcaaggaccgcgccggctccgcaaggaccgtggagcgcagagcaaatgctggtgtcgtcatggcagatgcccggcggcatctacgcgccgatgatgcatccctggtcgacgggcaaagacgtctactttaacctgtctttgtggtcggcatacaacgtgatgttgatgcacaccgtgctgcca

SEQ ID NO: 50 - DNA, 657 bp, Human metapneumovirus
  source 1..657: mol_type = genomic DNA
atggaggtgaaagtagagaacattcgagcaatagacatgctcaaagcaagagtgaaaaatcgtgtggcacgtagcaaatgctttaaaaatgcttctttaatcctcataggaataactacactgagtatagctctcaatatctatctgatcataaactacacaatacaaaaaacctcatctgaatcagaacaccacaccagctcaccacccacagaatccaacaaggaagcttcaacaatctccacagacaacccagacatcaatccaaactcacagcatccaactcaacagtccacagaaaaccccacactcaaccccgcagcatcagtgagcccatcagaaacagaaccagcatcaacaccagacacaacaaaccgcctgtcctccgtagacaggtccacagcacaaccaagtgaaagcagaacaaagacaaaaccgacagtccacacaagaaacaacccaagcacagcttccagtacacaatccccaccacgggcaacaacgaaggcaatccgcagagccaccactttccgcatgagcagcacaggaaaaagaccaaccacaacatcagtccagtccgacagcagcaccacaacccaaaatcatgaagaaacaggttcagcgaacccacaggcatctgtaagcacaatgcaaaac

SEQ ID NO: 51 - DNA, 1194 bp, Streptococcus pyogenes
  source 1..1194: mol_type = genomic DNA
atgaataagaaaaagctgggcgtgagactgctgagcctgctggccctgggtggtttcgtgctggccaaccccgtcttcgctgaccagaattttgcaaggaacgagaaagaagctaaggattcagcaatcacctttattcagaaaagcgccgctatcaaggcaggggctcgctctgccgaagacatcaagctggataaagtgaatctgggaggcgagctgtccggctctaatatgtacgtctataacatcagtactggcgggttcgtcatcgtgagcggggacaaacgaagtcctgaaatcctggggtacagtacctcaggcagcttcgacgccaatggaaaagagaacatcgcttcctttatggagtcttacgtggaacagattaaggagaacaaaaagctggacaccacctacgccggcacagccgaaatcaaacagccagtggtcaagagcctgctggactcaaagggcatccactacaaccagggcaatccatataacctgctcacacccgtgatcgaaaaagtgaagccaggggagcagtcctttgtcggacagcacgcagccactggttgcgtggcaactgccaccgctcagatcatgaagtaccataactacccgaacaaagggctgaaggactacacatatactctgagcagcaacaatccctacttcaatcatcctaagaacctgtttgctgcaatcagcaccaggcagtacaattggaacaatatcctgccaacatatagcggacgcgaatccaacgtgcagaaaatggcaatctccgagctgatggccgacgtgggaatttctgtggacatggattacggcccctctagtgggtccgcaggatcaagccgggtgcagcgggccctgaaggagaatttcgggtataaccagagtgtccaccagatcaataggggagacttttcaaaacaggattgggaagcccagattgataaggagctgtctcagaaccagcccgtgtactatcagggtgtgggcaaggtcggaggtcacgctttcgtgatcgacggtgcagatggccgaaacttctaccatgttaactggggttggggtggggtgagcgacgggttctttcggctggacgccctgaacccatccgctctgggcaccggaggtggtgcaggtggattcaacggctaccagtccgccgttgtgggcatcaagcct

SEQ ID NO: 52 - DNA, 1566 bp, Helicobacter pylori; identical to SEQ ID NO: 12
  source 1..1566: mol_type = genomic DNA

SEQ ID NO: 53 - DNA, 720 bp, Human rhinovirus A1
  source 1..720: mol_type = genomic DNA
atgggactgcccgtgtacatcaccccaggcagtgggcagtttatgaccacagacgatatgcagtcaccctgcgccctgccttggtaccacccaactaaggagatctctattcccggtgaagtgaaaaacctgatcgagatgtgccaggtcgacaccctgattcctgtgaacaatgtcggaaacaacgtgggtaacgtgagtatgtatactgtgcagctgggcaatcagaccgggatggctcagaaggtcttctccatcaaagttgatattacatctcagcctctcgccactaccctgatcggcgaaattgcttcctactatactcattggaccggctcactcaggttcagctttatgttctgcgggaccgctaatacaactctgaagctgctcctggcatacacaccccctgggatcgacgagccaaccacacgcaaagatgccatgctgggaacacacgtggtctgggacgtgggtctccagtctactattagtctggttgtgccctgggtcagcgcctcccattttcgactgacagctgataacaagtactctatggcaggctatatcacctgctggtaccagacaaacctggtcgttccaccgagcacacctcagactgcagacatgctgtgcttcgtgagcgcctgcaaagatttctgtctccggatggccagagacactgatctgcacatccagagcggcccaattgaacagttg

SEQ ID NO: 54 - DNA, 987 bp, Helicobacter pylori
  source 1..987: mol_type = genomic DNA
atgtcaaatagcatgttggataaaaataaagcgattcttacagggggtggggctttattattagggctaatcgtgcttttttatttagcttatcgccctaaggctgaagtgttgcaagggtttttggaagccagagaatacagcgtgagctccaaagtccctggccgcattgaaaaggtgtttgttaaaaaaggcgatcacattaaaaagggcgatttggtttttagcatttctagccctgaattagaagccaaactcgctcaagctgaagccgggcataaagccgctaaagcgcttagcgatgaagtcaaaagaggctcaagagacgaaacgattaattctgcgagagacgtttggcaagcagccaaatcccaagccactttagccaaagagacttataagcgcgttcaagatttgtatgataatggcgtggcgagcttgcaaaagcgcgatgaagcctatgcggcttatgaaagcactaaatacaacgagagcgcggcttaccaaaagtataaaatggctttagggggggcgagctctgaaagtaagattgccgctaaggctaaagagagcgcggctttagggcaagtgaatgaagtggagtcttatttaaaagacgtcaaagcgacagccccaattgatggggaagtgagtaacgtgcttttaagcggtggcgagcttagccctaagggttttcctgtggttttaatgatagatttaaaggatagttggttaaaaatcagcgtgcctgaaaagtatttgaacgagtttaaagtgggtaaggaatttgaaggctatatcccggcgttgaaaaaaagcacgaaattcagggtcaaatatttgagcgtgatgggggattttgcgacttggaaagcgacgaataattccaacacttacgacatgaaaagctatgaagtggaagccatacccttagaagagttggaaaattttagggtagggatgagcgtgttagttaccattaaacct

SEQ ID NO: 55 - DNA, 687 bp, Human parainfluenza virus 4a
  source 1..687: mol_type = genomic DNA
atgtctttcgagatcagtgttgaggaaatcgaggaactgattgaaaccggcaacctgaatattgatcacgctctgaaggagctcggggcaacatcccagagctccctgaacaaacccccttctcagtctagtaggactgagggcaacgacggcgggaccaagatcagccgtaatccagcaccagtggaagcaccagctcacacatcaactgcccagaggagccataacgaggaaaatgagagtgggcgccagaacctggattcactcagcatgatcagcaataagcctcagaccggcacactgctcatgggtagcgacacccagctgccatccccttctaaaacataccagggcctgatcctggacgcaaagaaaagagccctgaacgagccacggagagatcagaagatcactaacgaacacggatccatgaatgacacccgaatcttcaagcgtggcgagtaccggtatcaggaaagaggactgggttacacagagtctgaaatcaagaataccacaccaactccgcggcataggcgcgagtatagtatctcatgggtcaacggacggactaccattagcgaatggtgcaatccatgctgtgcccccgtgaagagcacagcttccgtcgagaaatgcacttgtggccggtgccctaaaatctgcgaactgtgcattagagatcca

SEQ ID NO: 56 - DNA, 1125 bp, Helicobacter pylori
  source 1..1125: mol_type = genomic DNA
atgcgtgagattatttctgatgggaatgaattagtcgctaaagcggcgattgaagtggggtgtcggttttttgggggctatcctatcacgccaagttcggatattatgcatgcgatgagcgtggctttacccaaatgcggcggtcattttatccaaatggaagatgaaatcagcgggattagcgtgtctttaggagcgagcatgagcgggacgaagtctatgacagcaagctctgggcctggtatttcattgaaagtggagcaaatcggttattctttcatggcggaaatccctttagtgatcgctgatgtgatgcgttcaggcccatcaaccggaatgcccactcgtgtggctcaaggcgatgtgaatttcttaagacaccccatacatggggattttaaagccgtcgcgctcgctcctgcgaatttagaagaagcttacaccgaaaccgttcgcgcgttcaatttggctgaaatgctcatgactcctgtattcttgctcatggatgaaaccgtggggcatatgtatggcaaggtgcaaatcccagatttagaagaagtgcaaaagatgactattaatcgtaaggaatttctgggcgataaaaaagactacaagccttatggggtcgcacaagacgagccggctgttttgaaccctttctttaaaggttatcgctaccatgtttcaggcttgcaccatgggcctattggctttcctactgaagacgctaaaattggtggggatttgattgacagattatttaataagattgaatccaagcaagacattatcaacgaaaatgaggaaatggatttagagggtgctgaaatcgttgttatcgcttacggttcggtttctttggcggttaaagaggccttgaaagattaccataaagaaagcaagcaaaaagtcggctttttcaggcctaaaaccttatggccaagcccggctaaacgcttgaaagaaataggggataaatacgaaaaaatccttgtgattgaattgaataaagggcagtatttagaagaaattgaaagggctatgcaaagaaaggtgcatttcttggggcaagccaatgggcgcacgatttcgcctaaacaaatcatcgcaaaattgaaggagctt

SEQ ID NO: 57 - DNA, 1011 bp, Human herpesvirus 4
  source 1..1011: mol_type = genomic DNA
atggcgagcccggaagagaggctcctagacgagctcaataacgtaattgtgtcatttctgtgtgactctgggtctctggaagtggagagatgctccggggcgcatgtgttctccaggggcagctcccaacccctctgcaccgtgaagctgcgccacggacagatttaccacctggagtttgtctacaagttcctggcctttaagctgaagaactgcaactacccctcctcgcccgtgtttgtgatatccaacaacggcctggccaccaccctgaggtgctttttgcacgagccgtcgggtctcagatcgggccagagcggcccttgcctgggtctctcaacggatgttgacctaccaaagaactccatcattatgctgggccaggatgacttcattaagttcaaaagccccctggtcttccctgctgagcttgatctcctgaaatctatggtggtctgccgggcctacatcacggaacaccggacgacgatgcagtttctggtgtttcaggccgccaacgcccagaaggcctcgcgggtcatggatatgattagtgatatgtctcagcaactgtctcggtctggtcaagtcgaggatacgggcgccagagtcacaggtggaggaggtcccaggcctggcgtcacgcactcggggtgtcttggggactcacacgttagggggcgcggtggttgggacttggataacttttcagaagctgagaccgaagacgaggcgagttacgctccttggagggacaaagactcgtggtcggaatccgaggcggcgccgtggaagaaggaactcgtgaggcaccccatccgcaggcaccggacacgcgagactcgccgtatgcgcgggagccattcacgggtggaacacgtgccccccgagacccgggagacggtggtggggggagcatggcgttattcttggcgcgccacaccttatctggcacgggtgctggctgtcacggccgtggccctgctcctgatgtttctgaggtggacctga

SEQ ID NO: 58 - DNA, 1227 bp, Human mastadenovirus D
  source 1..1227: mol_type = genomic DNA
atgaacactctgacctccgtggtcctgctctctctgctcgtcgcattctctcaggccggcatcattaacctgaatgtgagttggggaatgaacctgaccctggtgggtccaagcgacctgcctgtcacatggtacgatggaaagggtatgcagttttgcgacgggaacaccatcaagaatcctcagatcaagcactcctgtaaccagcagaacctgaccctgctgaacgctgataaatcacacgagcgtacttacctgggatatcggcatgacagcaagggcaaagtggattacaaggtcaccgtgatcccccctccaccggcaactagaaaaccactgagtgaaccgcattatgtgacagtcactatgggggacaacattactctggttggacccagcgacctgcctgtgacctggtacgatggcgaggggaacaagttctgcgatggagagaaagtcgaacacgcagagtttaatcatacctgtaacatccagaatctgacactgctcttcgtgaacctgactcacaatggagcctacattggttataacaaggacgggagcgatcgggaactgtatgaggtgagcgtgaagaccctgtttcagaatggagccggccagtccaaagtggaacagggcaacaaggggaaacccaataccgctcagtctggcgggaagaaaaccaagacagagcatagaaaccagagtcctaagaggaaatcaacaaacaatctgcagccaactcagctctacgtccgccccttcactaacgtgagcctgaccggcccccctaatgggaaggtcacttggtacgacggcgaactgaatgatccctgcgagcagaagtataaactgcgaaccttttgcaatcaacaaaacctgaccctgatcaacgtgagctccacatacgacggaatctactatggtacagacgagaaggataaagccaatcggtatagaatcaaggtcaacaccacaaatcacaaaactgttaagatcaagccacatacccgggaaccaccggctgtgcaggaaaagcagttcgagctgcaggacgcagagaccgatgagaacgaatcaaaaatccccagcgccacagtggctatcgttgtgggcgtcattgctgggttcgtgaccctgatcatcgtgttcatctgctacatttgctgtaggaagcgccctcgaagctataatcacatggtggatccactgctctcttttagttat

SEQ ID NO: 59 - DNA, 708 bp, Streptococcus pyogenes
  source 1..708: mol_type = genomic DNA
atgcggtacaactgcagatattctcacatcgataagaaaatctacagtatgatcatttgtctgtcatttctgctctacagcaatgtggtccaggcaaacagctataataccacaaaccgacacaacctggagagcctgtacaagcatgactctaacctgatcgaagccgatagcatcaagaacagccctgacatcgtgactagtcacatgctgaagtattcagttaaggataaaaacctgtcagtgttctttgagaaggactggatcagccaggagtttaaggacaaagaagtggacatctacgccctgtccgctcaggaggtgtgcgaatgtcccggcaaacggtatgaggccttcggcgggatcactctgaccaacagtgagaagaaagagatcaaggtgcccgtgaatgtctgggacaagagcaaacagcagccccctatgtttatcaccgtgaacaagccgaaagtcacagcccaggaggtggacatcaaggtgcggaaactgctgatcaagaagtacgacatctacaacaatcgcgaacagaagtactccaaaggcacagtgaccctggacctgaacagcgggaaggacatcgtgttcgatctgtactattttggaaacggcgacttcaacagcatgctcaaaatctactccaacaatgagaggattgattctacacagttccacgtggacgtgagcatcagc

SEQ ID NO: 60 - DNA, 1377 bp, Enterovirus D68
  source 1..1377: mol_type = genomic DNA
atgggggagatcgtgagtaacgaaaagtcaggaatgtgcattaatgccccagctaaaaccaagctgcagccgtctgtcttccaccaggtttttgagggctctaaagaacccgctgtgctgaacagtaaagaccctcggctgaagactgatttcgaggaagcaatctttagcaaatacaccggcaataagattatgctgatggacgagtacatggaggaagccgtggatcattatgtcgggtgtctggagcctctcgacatttcagtggatcccatccctctggaaaacgccatgtacggaatggagggtctggaagctctggacctgaccacatccgcaggtttcccatatctgctccagggcaagaaaaagagagacatctttaatcgacagacacgtgatacttctgagatgacaaaaatgctggaaaagtacggggttgacctgcccttcgtgaccttcgtgaaagatgagctgaggtcccgcgagaaggtggaaaaggggaagtctaggctgatcgaagctagcagcctgaacgactccgttgctatgcgcgtggcattcggaaatctgtatgccacttttcacaacaatcccggaactgccaccggtagtgctgtgggctgcgacccagatatcttctggtcaaagattccgatcctgctcgatggcgagattttcgcttttgactacacaggctatgatgcatccctgtctcctgtctggttcgcctgcctgaaaaaggttctgatcaagctcggctacacccaccagaccagcttcatcgactacctgtgccacagcgtgcacctgtacaaagatcgaaagtatgtcatcaacggcgggatgccaagcggctctagtgggacttccattttcaataccatgatcaacaacatcatcatccgcaccctgctgatcaaggtctacaaggggatcgacctggatcagtttaagatgattgcctacggagacgacgtgatcgctagttatccccacaaaattgaccctggactgctcgcagaggccgggaagcattatggactggtcatgacccccgccgacaaaggcacatccttcatcgatactaactgggaaaatgtcacattcctgaagcggtactttagagccgacgatcagtatccatttctgattcacccagtgatgccgatgaaagagattcatgaaagcatccgatggaccaaggacccccgtaacacacaggatcacgtgagatccctgtgctacctggcatggcataacggcgaggaagcctataatgagttctgtaggaagatccgctctgtgccagtcggacgggccctgaccctgcctgcatactcaagcctgcggagaaaatggctcgatagttttttg

SEQ ID NO: 61 - protein, 351 aa, Streptococcus gallolyticus
MKKKLVIVGALSALVLPLFLVVIIMGAFAGSASSNGQAKGLQTKLIAKEVAQKANISEERASDVIKILNYELSQEQFTIAGASGSLAVAERESGFDPKAVNTGGGVAGYFQWSGWSSTINGNRWSKAKQKKLDSDIELDLLSAELNSSYSKVKTEMQKTTDPEKAALYWSEHYEGVALSDGQTKASDLKKNARKWYNLFKDTLTGDTANAKTRDGGGVTSDGVPAGYSLTKAINTSNYTSATYPWGQCTWFVYNRAKEVGVNFDPYMGNGGDWKQKAGYQTTHTPTEHSALSFSPGQAGADPTYGHIAFVEQVKSDGSILISEANAKGLGVVSYRTFDKVTANQFTYVIGK

SEQ ID NO: 62 - protein, 237 aa, Pseudomonas aeruginosa
MFTSRCLPLAAAVTALALLAGCANNNPYDTQSQSQGGMSKTAKYGGLGALAGAVAGAAIDHNNRGKGALIGAAVAGAAAAGYGYYADKQEAELRRQMEGTGVEVQRQGDDIKLIMPGNITFATDSANIAPSFYAPLNNLANSFKQYNQNTIEIVGYTDSTGSRQHNMDLSQRRAQSVAGYLTAQGVDGTRLSTRGMGPDQPIASNSTADGRAQNRRVEVNLRPVPGAQGPAQTQPQY

SEQ ID NO: 63 - protein, 278 aa, Helicobacter pylori
MERSLIFKKVRIYSKMLVALGLSSVLIGCAMNPSAETKTPNDAKNQVQTHERMKTSSEHVTPLDFNYPIHIVQAPQNHHVVGILTPRIQVSDNLKPYIDKFQDALINQIQTIFEKRGYQVLRFQDEKALNAQDKRKIFSVLDLKGWVGILEDLKMNLKDPNNPNLDTLVDQSSGSVWFNFYEPESNRVVHDFAVEVGTFQAMTYTYKHNNSGGLNSSNSIIHEYLEKNKEDAIHKILNRMYAVVMKKAVTELTKENIDKYREAIDRMKGFKSSMPQKK

SEQ ID NO: 64 - protein, 570 aa, Human herpesvirus 4
MALSGHVLIDPARLPRDTGPELMWAPSLRNSLRVSPEALELAEREAERARSERWDRCAQVLKNRLLRVELDGIMRDHLARAEEIRQDLDAVVAFSDGLESMQVRSPSTGGRSAPAPPSPSPAQPFTRLTGNAQYAVSISPTDPPLMVAGSLAQTLLGNLYGNINQWVPSFGPWYRTMSANAMQRRVFPKQLRGNLNFTNSVSLKLMTEVVAVLEGTTQDFFSDVRHLPDLQAALILSVAYLLLQGGSSHQQRPLPASREELLELGPESLEKIIADLKAKSPGGNFMILTSGNKEARQSIAPLNRQAAYPPGTFADNKIYNLFVGAGLLPTTAALNVPGAAGRDRDLVYRIANQIFGEDVPPFSSHQWNLRVGLAALEALMLVYTLCETANLAEAATRRLHLSSLLPQAMQRRKPAMASAGMPGAYPVQTLFRHGELFRFIWAHYVRPTVAADPQASISSLFPGLVLLALELKLMDGQAPSHYAINLTGQKFDTLFEIINQKLLFHDPAAMLAARTQLRLAFEDGVGVALGRPSPMLAAREILERQFSASDDYDRLYFLTLGYLASPVAPS

SEQ ID NO: 65 - protein, 184 aa, Helicobacter pylori
MKKMVLVSVLLAGFLQAVNLDLSSAKLTWTAFKTKAKTPVNGSFESITYKLGKSQDSLKTLLEGASASMDSLKVNLGDELKNKNVKEAFFALFKNTNIKVTFRNVIEGDHAGSLTAYVRMNEKLVKVPMQYTIAEDKIVVKGVLDLLNFGLKNELASLAKRCESFHEGLTWSQVEIQFESMIKG

SEQ ID NO: 66 - protein, 355 aa, Helicobacter pylori
MSGISAQLVKKLRDLTDAGMMDCKKALVEVAGDLQKAIDFLREKGLSKAAKKADRIAAEGVVALEVAPDFKSAMIVEINSETDFVAKNEGFKELVKKTLETIKAHNIHTTEELLKSPLDNKPFEEYLHSQIAVIGENILVRKIAHLKAPSSHIINGYAHSNARVGVLIGIKYDNEKNAPKVVELARNIAMHAAAMKPQVLDCKDFSLDFVKKETLALIAEIEKDNEEAKRLGKPLKNIPTFGSRIELSDEVLAHQKKAFEDELKAQGKPEKIWDKIVPGKMERFIADNTLIDQRLTLLGQFYVMDDKKTIAQVVADCSKEWNDDLKITEYVRFELGEGIEKKAENFAEEVALQMK

SEQ ID NO: 67 - protein, 202 aa, Gemella haemolysans
MVVKYVDENGKDLIPAETTEGKEGDEYTTSGKVIPGYVLVRVDGEAKGKIGKDGSTVTYVYKPLGSWVTNIPGQPTSQIKYPNDPTDPTKPGSERPVLPYVPGYTPVDGNGNPLKPVDPQDPTKGYIIPDIPTNPGQDTVINYVANPKPQPKQDQKPVQPKANAQVKRLANTGATETNTGLAGLGLAALGGMLAAVRRRKEK

SEQ ID NO: 68 - protein, 185 aa, Streptococcus pneumoniae
MKKVMFAGLSLLSLVVLMACGEEETKKTQAAQQPKQQTTVQQIAVGKDAPDFTLQSMDGKEVKLSDFKGKKVYLKFWASWCGPCKKSMPELMELAAKPDRDFEILTVIAPGIQGEKTVEQFPQWFQEQGYKDIPVLYDTKATTFQAYQIRSIPTEYLIDSQGKIGKIQFGAISNADAEAAFKEMN

SEQ ID NO: 69 - protein, 484 aa, Streptococcus pyogenes
MAKNNTNRHYSLRKLKTGTASVAVALTVLGAGFANQTEVKANGDGNPREVIEDLAANNPAIQNIRLRYENKDLKARLENAMEVAGRDFKRAEELEKAKQALEDQRKDLETKLKELQQDYDLAKESTSWDRQRLEKELEEKKEALELAIDQASRDYHRATALEKELEEKKKALELAIDQASQDYNRANVLEKELETITREQEINRNLLGNAKLELDQLSSEKEQLTIEKAKLEEEKQISDASRQSLRRDLDASREAKKQVEKDLANLTAELDKVKEDKQISDASRQGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAGKASDSQTPDTKPGNKAVPGKGQAPQAGTKPNQNKAPMKETKRQLPSTGETANPFFTAAALTVMATAGVAAVVKRKEEN

SEQ ID NO: 70 - protein, 248 aa, Pseudomonas aeruginosa
MRRTVKGLSRMALLALVLALGGCKVELYTGISQKEGNEMLALLRSEGVSADKQADKDGTVRLLVEESDIAEAVEVLKRKGYPRENFSTLKDVFPKDGLISSPIEERARLNYAKAQEISHTLSEIDGVLVARVHVVLPEERDGLGRKSSPASASVFIKHAADVQLDAYVPQIKQLVNNGIEGLSYDRISVVLVPSAGVRQVPLAPRFESVFSIQVAEHSRGRLLGLFGLLLALLLASNLAQFFWHRQRG

SEQ ID NO: 71 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 72 - protein, 522 aa, Helicobacter pylori
MGQAFFKKIVGCFCLGYLFLSSAIEAAALDIKNFNRGRVKVVNKKIAYLGDEKPITIWTSLDNVTVIQLEKDETISYITTGFNKGWSIVPNSNHIFIQPKSVKSNLMFEKEAVNFALMTRDYQEFLKTKKLIVDAPDPKELEEQKKALEKEKEAKEQAQKAQKDKREKRKEERAKNRANLENLTNAMSNPQNLSNNKNLSEFIKQQRENELDQMERLEDMQEQAQANALKQIEELNKKQAEETIKQRAKDKINIKTDKPQKSPEDNSIELSPSDSAWRTNLVVRTNKALYQFILRIAQKDNFASAYLTVKLEYPQRHEVSSVIEEELKKREEAKRQKELIKQENLNTTAYINRVMMASNEQIINKEKIREEKQKIILDQAKALETQYVHNALKRNPVPRNYNYYQAPEKRSKHIMPSEIFDDGTFTYFGFKNITLQPAIFVVQPDGKLSMTDAAIDPNMTNSGLRWYRVNEIAEKFKLIKDKALVTVINKGYGKNPLTKNYNIKNYGELERVIKKLPLVRDK

SEQ ID NO: 73 - protein, 195 aa, Influenza A virus
MQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICIS

SEQ ID NO: 74 - protein, 271 aa, Helicobacter pylori
MNAFKRIISVGVIALGLFNLLDAKHHKEKKENHKITRELKVGANPVPHAQILQSVVDDLKEKGIKLVIVSFTDYVLPNLALNDGSLDANYFQHRPYLDRFNLDRKMHLVGLANIHVEPLRFYSQKITDIKNLKKGSVIAVPNDPANQGRALILLHKQGLIALKDPSNLYATEFDIVKNPYNIKIKPLEAALLPKVLGDVDGAIITGNYALQAKLTGALFSEDKDSPYANLIAAREDNAQDEAIKTLIEALQSEKTRKFILDTYKGAIIPAF

SEQ ID NO: 75 - protein, 219 aa, Influenza A virus
MDSNTMSSFQVDCFLWHIRKRFADNGLGDAPFLDRLRRDQKSLKGRGNTLGLDIETATLVGKQIVEWILKEESSETLRMTIASVPTSRYLSDMTLEEMSRDWFMLMPRQKIIGPLCVRLDQAIMEKNIVLKANFSVIFNRLETLILLRAFTEEGAIVGEISPLPSLPGHTYEDVKNAVGVLIGGLEWNGNTVRVSENIQRFAWRNCDENGRPSLPPEQK

SEQ ID NO: 76 - protein, 45 aa, Helicobacter pylori
MLKNMIFVLHEAFEFSYIFNGIFTHQKLVCSLFCQPSLLVDCCMR

SEQ ID NO: 77 - protein, 835 aa, Helicobacter pylori
MTEDFPKILPLLVEEDTFLYPFMIAPIFLQNNASIKAVAYAKNNKSLVFIACQKDKLNDNEAPYYDVGVIGSVMREANMPNGRVKLLFNGIAKGRILEPAKENEQGFLEAQISPIEYLEYDKENIQAIVEVLKEKVITLANVSSLFPPDLIKALEDNDDPNRIADLIAAALHLKKDQAYSLFANNNTEQRLLDLIDIVIEETKTQKLQKEIKSKVHQKMEQTNKEYFLKEQLKQIQKELGTDKQRDEDLNQYYQKLESIKPFLKEEAFKEIKKQIDRLSRTHADSSDSATLQNYIETMLDVPFGQYGKKALDIKHVREQLDKDHYSLKRPKERIVEYFATMQLLEMRRKKKPEKKDKTKGTILCFYGPPGVGKTSLANSIAKAIERPLVRIALGGLEDVNELRGHRRTYIGSMPGRIVQGLIEAKKMNPVMVLDEIDKVDRSVRGDPASALLEILDPEQNTAFRDHYANFSIDLSQVIFIATANNIDRIPAPLRDRMEFISVSSYTPNEKEEIAKNYLIPQELEKHALKPSEVEISHECLKLIIEKYTREAGVRDLRRQIATIMRKVALKYLEDNPHQKGRTKKGKNEKSEDQKSEDQKSENQKSENKDFCVSITPNNLKEYLERMVFEIDPIDEENKIGIVNGLAWTPVGGDVLKIEVLKIRGKGELKLTGSLGDVMKESAIIAFSVVKVLLDNETLKVPKIPSETDAEGKKKKKVLKVYNAYDLHLHVPEGATPKDGPSAGIAMASVMASILCDRATRSEVAMTGELTLSGEVLPIGGLKEKLIAAFKAGIKTALIPVKNYERDLDEIPAEVRENLNIVAVKNIAEVLEKTLL

SEQ ID NO: 78 - protein, 365 aa, Campylobacter jejuni
MFGSKINHSDLQKLEEENKNLTHKIEKFQSENLELKNKITSLEQAALESKLKTDLLNVLLTGVLKNITIIQGDMLENVNKAEVISSYSKTSLAEMDELNHIANSINASLGNITESANKTRDVAGTLHRSVDEITNVINLIKDVSDQTNLLALNAAIEAARAGEHGRGFAVVADEVRKLAEKTQKATTEVEMNINLLKQNANEMYTQSEQVEKISIDSNAHIMSFSEKFTHLVNEAHSTNSNAVGIASEAFVSLAKLDHIAFKLNGYKEIFSKSGKQLADHTSCRLGKWLASTGKERFGQNKSFLKINEPHEKVHENMNNAITIANTEDISKDITQHSIINKCEVAENASLDLFNVFKEMLDESDH

SEQ ID NO: 79 - protein, 207 aa, Streptococcus pyogenes
MKKGLLVTTGLACLGLLTACSTQDNMAKKEITQDKMSMAAKKKDKMSTSKDKSMMADKSSDKKMTNDGPMAPDFELKGIDGKTYRLSEFKGKKVYLKFWASWCSICLSTLADTEDLAKMSDKDYVVLTVVSPGHQGEKSEADFKKWFQGTDYKDLPVLLDPDGKLLEAYGVRSYPTEVFIGSDGVLAKKHIGYAKKSDIKKTLKGIH

SEQ ID NO: 80 - protein, 479 aa, Human herpesvirus 4
MVPSQRLSRTSSISSNEDPAESHILELEAVSDTNTDCDMDPMEGSEEHSTDGEISSSEEEDEDPTPAHAIPAQPSSVVITPTSASFVIPRKKWDLQDKTVTLHRSPLCRDEDEKEETGNSSYTRGHKRRRGEVHGCTDESYGKRRHLPPGARMPRAPRAPRMPRAPRSPRAPRSNRATRGPRSESRGAGRSTRKQARQERSQRPLPNKPWFDMSLVKPVSKITFVTLPSPLASLTLEPIQDPFLQSMLAVAAHPEIGAWQKVQPRHELRRSYKTLREFFTKSTNKDTWLDARMQAIQNAGLCTLVAMLEETIFWLQEITYHGDLPLAPAEDILLACAMSLSKVILTKLKELAPCFLPNTRDYNFVKQLFYITCATARQNKVVETLSSSYVKQPLCLLAAYAAVAPAYINAHCRRRHDEVEFLGHYIKNYNPGTLSSLLTEAVETHTRDCRSASCSRLVRAILSPGTGSLGLFFVPGLNQ

SEQ ID NO: 81 - protein, 693 aa, Streptococcus pneumoniae
MFASKSERKVHYSIRKFSVGVASVVVASLVMGSVVHATENEGATQVPTSSNRANESQAEQGEQPKKLDSERDKARKEVEEYVKKIVGESYAKSTKKRHTITVALVNELNNIKNEYLNKIVESTSESQLQILMMESRSKVDEAVSKFEKDSSSSSSSDSSTKPEASDTAKPNKPTEPGEKVAEAKKKVEEAEKKAKDQKEEDRRNYPTITYKTLELEIAESDVEVKKAELELVKVKANEPRDEQKIKQAEAEVESKQAEATRLKKIKTDREEAEEEAKRRADAKEQGKPKGRAKRGVPGELATPDKKENDAKSSDSSVGEETLPSPSLKPEKKVAEAEKKVEEAKKKAEDQKEEDRRNYPTNTYKTLELEIAESDVEVKKAELELVKEEAKEPRNEEKVKQAKAEVESKKAEATRLEKIKTDRKKAEEEAKRKAAEEDKVKEKPAEQPQPAPAPKAEKPAPAPKPENPAEQPKAEKPADQQAEEDYARRSEEEYNRLTQQQPPKTEKPAQPSTPKTGWKQENGMWYFYNTDGSMATGWLQNNGSWYYLNSNGAMATGWLQNNGSWYYLNANGSMATGWLQNNGSWYYLNANGSMATGWLQYNGSWYYLNANGSMATGWLQYNGSWYYLNANGDMATGWVKDGDTWYYLEASGAMKASQWFKVSDKWYYVNGSGALAVNTTVDGYGVNANGEWVN

SEQ ID NO: 82 - protein, 351 aa, Streptococcus gallolyticus; identical to SEQ ID NO: 61

SEQ ID NO: 83 - protein, 140 aa, Adenovirus
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAV

SEQ ID NO: 84 - protein, 580 aa, Human herpesvirus 5
MESSAKRKMDPDNPDEGPSSKVPRPETPVSKATTFLQTMLRKEVNSQLNLGDPLFPELAEESLKTFEQVTEDCNENPEKDVLAELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 85 - protein, 237 aa, Pseudomonas aeruginosa; identical to SEQ ID NO: 62

SEQ ID NO: 86 - protein, 289 aa, Human rhinovirus A1
MNPVENYIDEVLNEVLVVPNIKESHHTTSNSAPLLDAAETGHTSNVQPEDAIETRYVITSQTRDEMSIESFLGRSGCVHISRIKVDYTDYNGQDINFTKWKITLQEMAQIRRKFELFTYVRFDSEITLVPCIAGRGDDIGHIVMQYMYVPPGAPIPSKRNDFSWQSGTNMSIFWQHGQPFPRFSLPFLSIASAYYMFYDGYDGDNTSSKYGSVVTNDMGTICSRIVTEKQKHSVVITTHIYHKAKHTKAWCPRPPRAVPYTHSHVTNYMPETGDVTTAIVRRNTITTAL

SEQ ID NO: 87 - protein, 387 aa, Human adenovirus 41
MKRTRIEDDFNPVYPYDTFSTPSIPYVAPPFVSSDGLQEKPPGVLALKYTDPITTNAKHELTLKLGSNITLENGLLSATVPTVSPPLTNSNNSLGLATSAPIAVSANSLTLATAAPLTVSNNQLSINAGRGLVITNNAVAVNPTGALGFNNTGALQLNAAGGMRVDGANLILHVAYPFEAVNQLTLRLENGLEVTSGGKLNVKLGSGLQFDSNGRIAISNSNRTRSVPSLTTIWSISPTPNCSIYETQDANLFLCLTKNGAHVLGTITIKGLKGALREMHDNALSLKLPFDNQGNLLNCALESSTWRYQETDAVASNALTFMPNSTVYPRNKTADPGNMLIQISPNITFSVVYNEINSGYAFTFKWSAEPGKPFHPPTAVFCYITEQ

SEQ ID NO: 88 - protein, 460 aa, Streptococcus gallolyticus
MKKRILSAVLVSGVTLGTAAATVNADDYDTQIAAQDAVISNLTSEQAAAQSQVDALQEQVTSLQSQQDELEAQNAQLEAESQKLSEEIQALSSKIVARNESLKKQARSAQKTNTATSYINTILNSKSISDAINRVAAVREVVSANEKMLEQQEADKAAIEQKQAENQEAINTVAANKATIEQNQAALATQQAELEAAQLNLSAQLATAEDEKASLVAQKEAAEQAAAEAAAAQAAAEAQAQAEAEAQAASVAQAQESVENGTATVDTTTDTSSQDSTTASTDTAAATEDTSSTQQAATVTPTATTTTSSSSSSSSASSSSSSSSSASTSSTASTSTSSSSSSSSSSSSVNTYPVGQCTWGVKSLASWVGNNWGNANQWIASAQAAGHSVGTTPQVGAVAVWPYDGGGYGHVAYVTAVQSSTSIQVMEANYAGNSSIGNYRGWFDPTSSTWGGGTVYYIYQ

SEQ ID NO: 89 - protein, 570 aa, Human herpesvirus 4; identical to SEQ ID NO: 64

SEQ ID NO: 90 - protein, 318 aa, Streptococcus pneumoniae
MEINVSKLRTDLPQVGVQPYRQVHAHSTGNPHSTVQNEADYHWRKDPELGFFSHIVGNGCIMQVGPVDNGAWDVGGGWNAETYAAVELIESHSTKEEFMTDYRLYIELLRNLADEAGLPKTLDTGSLAGIKTHEYCTNNQPNNHSDHVDPYPYLAKWGISREQFKHDIENGLTIETGWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADKPEFTVEPDGLITVK

SEQ ID NO: 91 - protein, 196 aa, Helicobacter pylori
MDILDLNKAQAVQQNEQEVEDKERESKEPVVLEDLSALAWLELEEFSRLSGLPKERILELVNLGKIKSKISSNKLLIDASSGTNALIKKVENSLISMDMNGRSLEPVFVEKTINTILNLHDKVIGAKDETISAFKNENMFLKDALISMQEVYEEDKKTIDLLRDELNQAREEIEFMKRKYRLMWGKVADMSSVNKK

SEQ ID NO: 92 - protein, 700 aa, Helicobacter pylori
MLRFVSKTICLSLIGLFNPLEAFQKHQKDGFFIEAGFETGLLEGTQTKEEVITTQKIYENPLTHPQTKEQPKEQNKSDTATPQSAYGKYYIPQSTILKNATALFTTDKIENGLTFYSQNPVYANMVNGSVTIQNFLPYNLNNVELSFKDAQGKVVNLGVIETIPKQSQITLPASLFNDSEFEQADSFNYQQLQATATQFSDANTQSLFQKLSKITTNVTMSYENADTNNFKGNCHDCVSDFTPQTAEELTNLMLDMIAVFDSKSWEEAVLNAPFQFSNSSSECGSDFPKCVNPFNNGRVAPIYEKYVLTPQSVIDAFRRTINLEVNILKSGFVGLGYELDDNDGNLGIEASALNPEKLFGKTLNKVDIVELRDIIHEFSHTKGYTHNGNMTYQRVRLCQENGGAIQECEGGKEELVNGKEELKFTNGKEVKDQDGYTYDVCSFYKDNHQVYTASNYPNSIYTNCARVPAGLIGVTTAVWQQLINQNALPINFANLNSPTNHLNAGLNAQNFATSIVSAIAQNFSTTSTTTYRSSSKNFRSPILGVNVKIGYQHYFNDYIGLAYYGIIKYNYAKTNDEKIQQLSYGGGMDVLFDFITTYANKKQDNPTKKAFASSFGVFGGLRGLYNSYYVFNQVKGSGNLDIVTGFNYRYKHSKYSVGISVPLIQSGIKIASNNGIYANSVVLNEGGSHFKVFFNYGWIF

SEQ ID NO: 93 - protein, 265 aa, Human mastadenovirus A
MEDINFSSLAPRHGTRPYMGTWNEIGTSQLNGGAFNWNSIWSGLKNFGSTIKTYGTKAWNSQTGQMLRDKLKDQNFQQKVVDGLASGINGVVDIANQAVQKKIANRLEPRPDEVMVEEKLPPLETVPGSVPTKGEKRPRPDAEETLVTHTTEPPSYEEAIKQGAALSPTTYPMTKPILPMATRVYGKNENVPMTLELPPLPEPTIADPVGSVPVASVPVASTVSRPAVRPVAVASLRNPRSSNWQSTLNSIVGLGVKSLKRRRCY

SEQ ID NO: 94 - protein, 149 aa, Human mastadenovirus C
MRADREELDLPPPIGGVAIDVVKVEVPATGRTLVLAFVKTCAVLAAVHGLYILHEVDLTTAHKEAEWEFEPLAWRVWLVVFYFGCLSLTVWLLEGSYGGSDHHAARAQSPDVRARRSELDDNIAQMGAVHGLELPRRQVRRELLQVYLA

SEQ ID NO: 95 - protein, 603 aa, Mycobacterium tuberculosis
MSFVIAAPETLVRAASDLANIGSTLGAANAAALGPTTELLAAGADEVSAAIASLFAAHGQAYQAVSAQMSAFHAQFVQTFTAGAGAYASAEAAAAAPLEGLLNIVNTPTQLLLGRPLIGNGANGAPGTGQAGGAGGLLYGNGGAGGSGAPGQAGGPGGAAGLFGNGGAGGAGGDGPGNGAAGGAGGAGGLLFGSGGAGGPGGVGNTGTGGLGGDGGAAGLFGAGGIGGAGGPGFNGGAGGAGGRSGLFEVLAAGGAGGTGGLSVNGGTGGTGGTGGGGGLFSNGGAGGAGGFGVSGSAGGNGGTGGDGGIFTGNGGTGGTGGTGTGNQLVGGEGGAGGAGGNAGILFGAGGIGGTGGTGLGAPDPGGTGGKGGVGGIGGAGALFGPGGAGGTGGFGASSADQMAGGIGGSGGSGGAAKLIGDGGAGGTGGDSVRGAAGSGGTGGTGGLIGDGGAGGAGGTGIEFGSVGGAGGAGGNAAGLSGAGGAGGAGGFGETAGDGGAGGNAGLLNGDGGAGGAGGLGIAGDGGNGGKGGKAGMVGNGGDGGAGGASVVANGGVGGSGGNATLIGNGGNGGNGGVGSAPGKGGAGGTAGLLGLNGSPGLS

SEQ ID NO: 96 - protein, 293 aa, Streptococcus pyogenes
MLTFGGASAVKAEENEKVREQEKLIQQLSEKLVEINDLQTLNGDKESIQSLVDYLTRRGKLEEEWMEYLNSGIQRKLFVGPKGPAGEKGEQGPTGKQGERGETGPAGPRGDKGETGDKGAQGPVGPAGKDGQNGKDGLPGKDGKDGQNGKDGLPGKDGKDGQDGKDGLPGKDGKDGQDGKDGLPGKDGQPGKPAPKTPEVPQNPDTAPHTPKTPRIPGQSKDVTPAPQNPSNRGLNKPQTQGGNQLAKTPAAHDTHRQLPATGETTNPFFTAAAVAIMTTAGVVAVAKRQENN

SEQ ID NO: 97 - protein, 369 aa, Helicobacter pylori
MQFQKALLHSSFFLPLFLSFCIAEENGAYASVGFEYSISHAVEHNNPFLNQERIQIISNAQNKIFKLHQVKNEITSMPKTFAYINNALKNNSKLTPTEMQAEQYYLQSTFQNIEKIVMLSGGVSSNPQLVQALEKMQEPITNPLEFEENLRNLEVQFAQSQNRMLSSLSSQIAAISNSLNALDPNSYSKNISSMYGVSLSVGYKHFFTKKKNQGLRYYLFYDYGYTNFGFVGNGFDGLGKMNNHLYGLGIDYLYNFIDNAKKHSSVGFYLGFALAGSSWVGSGLSMWVSQTDFINNYLTGYQAKMHTSFFQIPLNFGVRVNVNRHNGFEMGLKIPLAMNSFYETHGKGLNTSLFFKRLVMFNVSYVYSF

SEQ ID NO: 98 - protein, 524 aa, Human parainfluenza virus 1
MAGLLSTFDTFSSRRSESINKSGGGAIIPGQRSTVSVFTLGPSVTDDADKLLIATTFLAHSLDTDKQHSQRGGFLVSLLAMAYSSPELYLTTNGVNADVKYVIYNIERDPKRTKTDGFIVKTRDMEYERTTEWLFGPMINKNPLFQGQRENADLEALLQTYGYPACLGAIIVQVWIVLVKAITSSAGLRKGFFNRLEAFRQDGTVKSALVFTGDTVEGIGAVMRSQQSLVSLMVETLVTMNTSRSDLTTLEKNIQIVGNYIRDAGLASFMNTIKYGVETKMAALTLSNLRPDINKLRSLVDIYLSKGARAPFICILRDPVHGDFAPGNYPALWSYAMGVAVVQNKAMQQYVTGRTYLDMEMFLLGQAVAKDADSKISSALEEELGVTDTAKERLRHHLTNLSGGDGAYHKPTGGGAIEVAIDHTDITFGVEDTADRDNKNWTNDSNERWMNHSISNHTITIRGAEELEEETNDEDITDIENKIARRLADRKQRLSQANNKRDTSSDADYENDDDATAAAGIGGI

SEQ ID NO: 99 - protein, 420 aa, Human herpesvirus 4
MVDEQVAVEHGTVSHTISREEDGVVHERRVLASGERVEVFYKAPAPRPREGRASTFHDFTVPAAAAVPGPEPEPEPHPPMPIHANGGGETKTNTQDQNQNQTTRTRTNAKAEERTAEMDDTMASSGGQRGAPISADLLSLSSLTGRMAAMAPSWMKSEVCGERMRFKEDVYDGEAETLAEPPRCFMLSFVFIYYCCYLAFLALLAFGFNPLFLPSFMPVGAKVLRGKGRDFGVPLSYGCPTNPFCKVYTLIPAVVINNVTYYPNNTDSHGGHGGFEAAALHVAALFESGCPNLQAVTNRNRTFNVTRASGRVERRLVQDMQRVLASAVVVMHHHCHYETYYVFDGVGPEFGTIPTPCFKDVLAFRPSLVTNCTAPLKTSVKGPNWSGAAGGMKRKQCRVDRLTDRSFPAYLEEVMYVMVQ

SEQ ID NO: 100 - protein, 266 aa, Streptococcus pneumoniae
MRKQIHPLILFSFFGIMMFILIINRPLNDSQSFKTKSNIAQIEAQALKHLDKPIIDLSGWQRPEEINYDALSQNISGAIVRVHSGAQTTKENDASFINGIDKAYKSHITELQKRNVPVAVYAYVAGKSVQEMEKAAEVFYNAASPYSPSYYWLDVEDKTMSNMNEGVENFRAKLASLGAKNIGIYVGVYFMEEHSIDTGKFTSVWIPSYGSDSGFLESSPKTDLDYDIHQYTSKGKIAGFDHDLDINVISPLKNKEETFRKLFLKP

SEQ ID NO: 101 - protein, 566 aa, Influenza A virus
MKTIIALSYILCLVFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 102 - protein, 115 aa, Human mastadenovirus C
MGETWFLTPNGQSSPGSWNARPSAGPAARMPTPRNRYFSRPSSTPSKVCTAPRAAPPPRASCAPRATPRRGWTMTPWSNATWPTRRAKTGSASAPAGPASSAPWPARSPERTPSP

SEQ ID NO: 103 - protein, 923 aa, Pseudomonas aeruginosa
VSLSTTAFPSLQGENMSRSPIPRHRALLAGFCLAGALSAQAATQEEILDAALVSGDSSQLTDSHLVALRLQQQVERIRQTRTQLLDGLYQNLSQAYDPGAASMWVLPANPDNTLPFLIGDKGRVLASLSLEAGGRGLAYGTNVLTQLSGTNAAHAPLLKRAVQWLVNGDPGAATAKDFKVSVVGVDKTAALNGLKSAGLQPADAACNALTDASCASTSKLLVLGNGASAASLSATVRARLQAGLPILFVHTNGWNQSSTGQQILAGLGLQEGPYGGNYWDKDRVPSSRTRTRSVELGGAYGQDPALVQQIVDGSWRTDYDWSKCTSYVGRTTCDDVPGLSDFSKRVDVLKGALDAYNQKAQNLFALPGTTSLRLWLLWADAVRQNIRYPMDKAADTARFQETFVADAIVGYVREAGAAQKELGSYAGQRQQSMPVSGSEETLTLTLPSAQGFTAIGRMAAPGKRLSIRIEDAGQASLAVGLNTQRIGSTRLWNTRQYDRPRFLKSPDIKLQANQSVALVSPYGGLLQLVYSGATPGQTVTVKVTGAASQPFLDIQPGEDSSQAIADFIQALDADKADWLEIRSGSVEVHAKVEKVRGSIDKDYGGDVQRFIRELNEVFIDDAYTLAGFAIPNQAKTPAIQQECAARGWDCDSETLHKLPGTQHINVDQYAQCGGGCSGNPYDQTWGLNPRGWGESHELGHNLQVNRLKVYGGRSGEISNQIFPLHKDWRVLREFGQNLDDTRVNYRNAYNLIVAGRAEADPLAGVYKRLWEDPGTYALNGERMAFYTQWVHYWADLKNDPLQGWDIWTLLYLHQRQVDKSDWDANKAALGYGTYAQRPGNSGDASSTDGNDNLLLGLSWLTQRDQRPTFALWGIRTSAAAQAQVAAYGFAEQPAFFYANNRTNEYSTVKLLDMSQGSPAWPFP

SEQ ID NO: 104 - protein, 149 aa, Human mastadenovirus C; identical to SEQ ID NO: 94

SEQ ID NO: 105 - protein, 693 aa, Streptococcus pneumoniae; identical to SEQ ID NO: 81

SEQ ID NO: 106 - protein, 242 aa, Influenza C virus
MAHEILIAETEAFLKNVAPETRTAIISAITGGKSACKSAAKLIKNEHLPLMSGEATTMHIVMRCLYPEIKPWKKASDMLNKATSSLKKSEGRDIRKQMKAAGDFLGVESMMKMRAFRDDQIMEMVEEVYDHPDDYTPDIRIGTITAWLRCKNKKSERYRSNVSESGRTALKIHEVRKASTAMNEIAGITGLGEEALSLQRQTESLAILCNHTFGSNIMRPHLEKAIKGVEGRVGEMGRMAMK

SEQ ID NO: 107 - protein, 559 aa, Human mastadenovirus D
MQQAPDPAIRAALQSQPSGLASDDWEAAMQRIMALTTRNPESFRQQPQANRLSAILEAVVPSRTNPTHEKVLAIVNALAENKAIRPDEAGLVYNALLERVGRYNSTNVQSNLDRLVTDVREAVAQRERFKNEGLGSLVALNAFLATQPANVPRGQDDYTNFISALRLMVTEVPQSEVYQSGPDYFFQTSRQGLQTVNLSQAFKNLRGLWGVQAPVGDRSTVSSLLTPNSRLLLLLIAPFTDSGSVNRNSYLGHLLTLYREAIGQAQVDEQTFQEITSVSRALGQNDTDSLRATLNFLLTNRQQKIPAQYALSAEEERILRYVQQSVGLFLMQEGATPSAALDMTARNMEPSMYAANRPFINKLMDYLHRAAVMNTDYFTNAILNPHWLPPPGFYTGEYDMPDPNDGFLWDDVDSVVFSPTFQKRQEAPPSEGAVGRSPFPSLGSLHSLPGSVNSGRVSRPRLLGEDEYLNDSLLQPPRAKNAMANNGIESLVDKLNRWKTYAQDHRDAPAPRRQRHDRQRGLVWDDEDSADDSSVLDLGGSGGVNPFAHLQPKLGRRMF

SEQ ID NO: 108 - protein, 700 aa, Helicobacter pylori; identical to SEQ ID NO: 92

SEQ ID NO: 109 - protein, 563 aa, Mycobacterium tuberculosis
MYRYQVRVQQRRSEMNRWVATRSRRHTYQWITDHKSPRDHYRHISELRTSIATSSPGRCDMSPIPRIVSVSLAWAAAIGLMVPIGLAPPAMAAPCSGDAANAPPPPSAIVTDPGATALGPVRPGHGPIPTGRKPRGANDRAPLPKLGPLISALLNPGARNAAPLQQQALVPRANPGPNPAPNPPATGPQPPNATQLTPNPAPAPDPAPAAAPDPGATLAGATTSLAEWVTGPDSPNKTLERFGISGTDLGIPWDNGDPANRQVLMIFGDTFGYCAVDGHQWRYNTLFRSQDRDLGNGVHVTSGDASNRYSGSPVRQPGFSKQLINSIKWARDETGIIPTAGIAVGKTQYVNFMSIRNWGRDGEWTTNYSGIAVSKDNGQTWGVFPGTIRASGPDSGGKARFVPGNENFQMGAYLKSNDGYLYSFGTPPGRGGSAYLARVPQRFVPDLTKYQYWNGDSNSWVPNKPDAATPVIPGPVGEMSVQYNTYLKQYLALYTNGMNDVVARTAPAPQGPWSAEQMLVSSWQMPGGIYAPMMHPWSTGKDVYFNLSLWSAYNVMLMHTVLP

SEQ ID NO: 110 - protein, 219 aa, Human metapneumovirus
MEVKVENIRAIDMLKARVKNRVARSKCFKNASLILIGITTLSIALNIYLIINYTIQKTSSESEHHTSSPPTESNKEASTISTDNPDINPNSQHPTQQSTENPTLNPAASVSPSETEPASTPDTTNRLSSVDRSTAQPSESRTKTKPTVHTRNNPSTASSTQSPPRATTKAIRRATTFRMSSTGKRPTTTSVQSDSSTTTQNHEETGSANPQASVSTMQN

SEQ ID NO: 111 - protein, 398 aa, Streptococcus pyogenes
MNKKKLGVRLLSLLALGGFVLANPVFADQNFARNEKEAKDSAITFIQKSAAIKAGARSAEDIKLDKVNLGGELSGSNMYVYNISTGGFVIVSGDKRSPEILGYSTSGSFDANGKENIASFMESYVEQIKENKKLDTTYAGTAEIKQPVVKSLLDSKGIHYNQGNPYNLLTPVIEKVKPGEQSFVGQHAATGCVATATAQIMKYHNYPNKGLKDYTYTLSSNNPYFNHPKNLFAAISTRQYNWNNILPTYSGRESNVQKMAISELMADVGISVDMDYGPSSGSAGSSRVQRALKENFGYNQSVHQINRGDFSKQDWEAQIDKELSQNQPVYYQGVGKVGGHAFVIDGADGRNFYHVNWGWGGVSDGFFRLDALNPSALGTGGGAGGFNGYQSAVVGIKP

SEQ ID NO: 112 - protein, 522 aa, Helicobacter pylori; identical to SEQ ID NO: 72

SEQ ID NO: 113 - protein, 240 aa, Human rhinovirus A1
MGLPVYITPGSGQFMTTDDMQSPCALPWYHPTKEISIPGEVKNLIEMCQVDTLIPVNNVGNNVGNVSMYTVQLGNQTGMAQKVFSIKVDITSQPLATTLIGEIASYYTHWTGSLRFSFMFCGTANTTLKLLLAYTPPGIDEPTTRKDAMLGTHVVWDVGLQSTISLVVPWVSASHFRLTADNKYSMAGYITCWYQTNLVVPPSTPQTADMLCFVSACKDFCLRMARDTDLHIQSGPIEQL

SEQ ID NO: 114 - protein, 329 aa, Helicobacter pylori
MSNSMLDKNKAILTGGGALLLGLIVLFYLAYRPKAEVLQGFLEAREYSVSSKVPGRIEKVFVKKGDHIKKGDLVFSISSPELEAKLAQAEAGHKAAKALSDEVKRGSRDETINSARDVWQAAKSQATLAKETYKRVQDLYDNGVASLQKRDEAYAAYESTKYNESAAYQKYKMALGGASSESKIAAKAKESAALGQVNEVESYLKDVKATAPIDGEVSNVLLSGGELSPKGFPVVLMIDLKDSWLKISVPEKYLNEFKVGKEFEGYIPALKKSTKFRVKYLSVMGDFATWKATNNSNTYDMKSYEVEAIPLEELENFRVGMSVLVTIKP

SEQ ID NO: 115 - protein, 229 aa, Human parainfluenza virus 4a
MSFEISVEEIEELIETGNLNIDHALKELGATSQSSLNKPPSQSSRTEGNDGGTKISRNPAPVEAPAHTSTAQRSHNEENESGRQNLDSLSMISNKPQTGTLLMGSDTQLPSPSKTYQGLILDAKKRALNEPRRDQKITNEHGSMNDTRIFKRGEYRYQERGLGYTESEIKNTTPTPRHRREYSISWVNGRTTISEWCNPCCAPVKSTASVEKCTCGRCPKICELCIRDP

SEQ ID NO: 116 - protein, 375 aa, Helicobacter pylori
MREIISDGNELVAKAAIEVGCRFFGGYPITPSSDIMHAMSVALPKCGGHFIQMEDEISGISVSLGASMSGTKSMTASSGPGISLKVEQIGYSFMAEIPLVIADVMRSGPSTGMPTRVAQGDVNFLRHPIHGDFKAVALAPANLEEAYTETVRAFNLAEMLMTPVFLLMDETVGHMYGKVQIPDLEEVQKMTINRKEFLGDKKDYKPYGVAQDEPAVLNPFFKGYRYHVSGLHHGPIGFPTEDAKIGGDLIDRLFNKIESKQDIINENEEMDLEGAEIVVIAYGSVSLAVKEALKDYHKESKQKVGFFRPKTLWPSPAKRLKEIGDKYEKILVIELNKGQYLEEIERAMQRKVHFLGQANGRTISPKQIIAKLKEL

SEQ ID NO: 117 - protein, 336 aa, Human herpesvirus 4
MASPEERLLDELNNVIVSFLCDSGSLEVERCSGAHVFSRGSSQPLCTVKLRHGQIYHLEFVYKFLAFKLKNCNYPSSPVFVISNNGLATTLRCFLHEPSGLRSGQSGPCLGLSTDVDLPKNSIIMLGQDDFIKFKSPLVFPAELDLLKSMVVCRAYITEHRTTMQFLVFQAANAQKASRVMDMISDMSQQLSRSGQVEDTGARVTGGGGPRPGVTHSGCLGDSHVRGRGGWDLDNFSEAETEDEASYAPWRDKDSWSESEAAPWKKELVRHPIRRHRTRETRRMRGSHSRVEHVPPETRETVVGGAWRYSWRATPYLARVLAVTAVALLLMFLRWT

SEQ ID NO: 118 - protein, 409 aa, Human mastadenovirus D
MNTLTSVVLLSLLVAFSQAGIINLNVSWGMNLTLVGPSDLPVTWYDGKGMQFCDGNTIKNPQIKHSCNQQNLTLLNADKSHERTYLGYRHDSKGKVDYKVTVIPPPPATRKPLSEPHYVTVTMGDNITLVGPSDLPVTWYDGEGNKFCDGEKVEHAEFNHTCNIQNLTLLFVNLTHNGAYIGYNKDGSDRELYEVSVKTLFQNGAGQSKVEQGNKGKPNTAQSGGKKTKTEHRNQSPKRKSTNNLQPTQLYVRPFTNVSLTGPPNGKVTWYDGELNDPCEQKYKLRTFCNQQNLTLINVSSTYDGIYYGTDEKDKANRYRIKVNTTNHKTVKIKPHTREPPAVQEKQFELQDAETDENESKIPSATVAIVVGVIAGFVTLIIVFICYICCRKRPRSYNHMVDPLLSFSY

SEQ ID NO: 119 - protein, 236 aa, Streptococcus pyogenes
MRYNCRYSHIDKKIYSMIICLSFLLYSNVVQANSYNTTNRHNLESLYKHDSNLIEADSIKNSPDIVTSHMLKYSVKDKNLSVFFEKDWISQEFKDKEVDIYALSAQEVCECPGKRYEAFGGITLTNSEKKEIKVPVNVWDKSKQQPPMFITVNKPKVTAQEVDIKVRKLLIKKYDIYNNREQKYSKGTVTLDLNSGKDIVFDLYYFGNGDFNSMLKIYSNNERIDSTQFHVDVSIS

SEQ ID NO: 120 - protein, 459 aa, Enterovirus D68
MGEIVSNEKSGMCINAPAKTKLQPSVFHQVFEGSKEPAVLNSKDPRLKTDFEEAIFSKYTGNKIMLMDEYMEEAVDHYVGCLEPLDISVDPIPLENAMYGMEGLEALDLTTSAGFPYLLQGKKKRDIFNRQTRDTSEMTKMLEKYGVDLPFVTFVKDELRSREKVEKGKSRLIEASSLNDSVAMRVAFGNLYATFHNNPGTATGSAVGCDPDIFWSKIPILLDGEIFAFDYTGYDASLSPVWFACLKKVLIKLGYTHQTSFIDYLCHSVHLYKDRKYVINGGMPSGSSGTSIFNTMINNIIIRTLLIKVYKGIDLDQFKMIAYGDDVIASYPHKIDPGLLAEAGKHYGLVMTPADKGTSFIDTNWENVTFLKRYFRADDQYPFLIHPVMPMKEIHESIRWTKDPRNTQDHVRSLCYLAWHNGEEAYNEFCRKIRSVPVGRALTLPAYSSLRRKWLDSFL